SEQUENCE LISTING (251 sequences)

SEQ ID NO: 1 - RNA, 86 nt, Homo sapiens
  hsa-mir-323 precursor
uugguacuuggagagaggugguccguggcgcguucgcuuuauuuauggcgcacauuacacggucgaccucuuugcaguaucuaauc

SEQ ID NO: 2 - RNA, 99 nt, Homo sapiens
  hsa-mir-342 precursor
gaaacugggcucaaggugaggggugcuaucugugauugagggacaugguuaauggaauugucucacacagaaaucgcacccgucaccuuggccuacuua

SEQ ID NO: 3 - RNA, 87 nt, Homo sapiens
  hsa-mir-520f precursor
ucucaggcugugacccucuaaagggaagcgcuuucuguggucagaaagaaaagcaagugcuuccuuuuagaggguuaccguuuggga

SEQ ID NO: 4 - RNA, 85 nt, Homo sapiens
  hsa-miR-3157 precursor
gggaagggcuucagccaggcuagugcagucugcuuugugccaacacuggggugaugacugcccuagucuagcugaagcuuuuccc

SEQ ID NO: 5 - RNA, 88 nt, Homo sapiens
  hsa-miR-193a precursor
cgaggaugggagcugagggcugggucuuugcgggcgagaugagggugucggaucaacuggccuacaaagucccaguucucggcccccg

SEQ ID NO: 6 - RNA, 110 nt, Homo sapiens
  hsa-miR-7-1 precursor
uuggauguuggccuaguucuguguggaagacuagugauuuuguuguuuuuagauaacuaaaucgacaacaaaucacagucugccauauggcacaggccaugccucuacag

SEQ ID NO: 7 - RNA, 110 nt, Homo sapiens
  hsa-miR-7-2 precursor
cuggauacagaguggaccggcuggccccaucuggaagacuagugauuuuguuguugucuuacugcgcucaacaacaaaucccagucuaccuaauggugccagccaucgca

SEQ ID NO: 8 - RNA, 110 nt, Homo sapiens
  hsa-miR-7-3 precursor
agauuagaguggcuguggucuagugcuguguggaagacuagugauuuuguuguucugauguacuacgacaacaagucacagccggccucauagcgcagacucccuucgac

SEQ ID NO: 9 - DNA, 483 bp, Artificial sequence
  hsa-mir-323 DNA sequence screening
ttcctggtatttgaagatgcggttgaccatggtgtgtacgctttatttgtgacgtaggacacatggtctacttcttctcaatatcacatctcgccttggaagacttccaggaggtgatatcagctttgcggaagagccactgtcctggtgtcagtacggctgctgcttggtacttggagagaggtggtccgtggcgcgttcgctttttttatggcgcacattacacggtcgacctctttgcagtatctaatcccgccttgcaagctttcctggagctaacatcaactgcgggggtgggggccactaggtctgcgctcagtgcgacccagcggggtttgtgatgtgtctgtcttgtgtgtgacgataactcacgtgtggcagccctcttctcagcacactgctctggcttggcagcagggttaacttgcggacgaggagcgtggtgtcagcacgtgcctggatacatgagatggttgaccagag

SEQ ID NO: 10 - DNA, 488 bp, Artificial sequence
  hsa-mir-342 DNA sequence screening
cctgaagagagactgacacatcagaggtgtcyggtgactgaacaagctcccagcttgcgcccatgtcatattgtgtgcctctcatagcctggcacttcctgccattgcatccttctctgcagactaagatggagttcctgaaccaagaccgcttgctggccaacctgtgaaactgggctcaaggtgaggggtgctatctgtgattgagggacatggttaatggaattgtctcacacagaaatcgcacccgtcaccttggcctacttatcaccaccccaaacagaggaacacgccttctccagccacagcctatggaagggccttcagctgctgtggccccgaggtgtgcatactgtggaaggaacttcggacgtgaactcggatctggttccagtaccagctgtgccaggagtgcccttgggcatgtcactgacctaagactcagtttcgccatctgtgaaatggctgaatcagactcacctcacagg

SEQ ID NO: 11 - DNA, 214 bp, Artificial sequence
  hsa-mir-520f DNA sequence screening
tgtgtccatttaaacctggtcaaggaagattcccacaaaaaatccacggtgctggagcaagaggatctcaggctgtgaccctctaaagggaagcgctttctgtggtcagaaagaaaagcaagtgcttccttttagagggttaccgtttgggaaaagcaatgttgaagttgatgctgatcttggtaaaatatttgcagagcgtgcttatcatcag

SEQ ID NO: 12 - DNA, 240 bp, Artificial sequence
  hsa-miR-3157 DNA sequence screening
acaacttctcaatgagtctgccctcactgtccaacaattgagctgagaatataagaagggaagggcttcagccaggctagtgcagtctgctttgtgccaacactggggtgatgactgccctagtctagctgaagcttttcccttctttctacacccagctcaagtcccaggtccataaaacctttagaaactcttcagaaactctttagagcttcagaagctcttgagaattggaagatg

SEQ ID NO: 13 - DNA, 294 bp, Artificial sequence
  hsa-miR-193a DNA sequence screening
agggacacccagagcttcggcggagcggagcgcggtgcacagagccggcgaccggacccagccccgggaagcccgtcggggacgcaccccgaactccgaggatgggagctgagggctgggtctttgcgggcgagatgagggtgtcggatcaactggcctacaaagtcccagttctcggcccccgggaccagcgtcttctccccggtcctcgccccaggccggcttcctcccgggctggcgtgcgctccggccaggctgcctctcaggtccacgctggagaaggagtggtgaggt

SEQ ID NO: 14 - DNA, 255 bp, Artificial sequence
  hsa-miR-7-1 DNA sequence screening
gccttaaccaagcaaacttctcatttctctggtgaaaactgctgccaaaaccacttgttaaaaattgtacagagcctgtagaaaatatagaagattcattggatgttggcctagttctgtgtggaagactagtgattttgttgtttttagataactaaatcgacaacaaatcacagtctgccatatggcacaggccatgcctctacaggacaaatgattggtgctgtaaaatgcagcatttcacaccttactagc

SEQ ID NO: 15 - DNA, 239 bp, Artificial sequence
  hsa-miR-7-2 DNA sequence screening
tgaaggagcatccagaccgctgacctggtggcgaggggaggggggtggtcctcgaacgccttgcagaactggcctggatacagagtggaccggctggccccatctggaagactagtgattttgttgttgtcttactgcgctcaacaacaaatcccagtctacctaatggtgccagccatcgcagcggggtgcaggaaatgggggcagcccccctttttggctatccttccacgtgttct

SEQ ID NO: 16 - DNA, 282 bp, Artificial sequence
  hsa-miR-7-3 DNA sequence screening
tcatagcttggctcaggtgagaaggaggagctgggcaggggtctcagacatggggcagagggtggtgaagaagattagagtggctgtggtctagtgctgtgtggaagactagtgattttgttgttctgatgtactacgacaacaagtcacagccggcctcatagcgcagactcccttcgaccttcgccttcaatgggctggccagtgggggagaaccggggaggtcggggaagaatcgcttccactcggagtgggggggctggctcactccaggcgatacag

SEQ ID NO: 17 - RNA, 7 nt, Homo sapiens
  hsa-miR-323-5p seed
ggugguc

SEQ ID NO: 18 - RNA, 7 nt, Homo sapiens
  hsa-miR-342-5p seed
ggggugc

SEQ ID NO: 19 - RNA, 7 nt, Homo sapiens
  has-miR-520f-3p  seed
agugcuu

SEQ ID NO: 20 - RNA, 7 nt, Homo sapiens
  hsa-mir-520f-3p-i3 seed
aagugcu

SEQ ID NO: 21 - RNA, 7 nt, Homo sapiens
  hsa-miR-3157-5p seed
ucagcca

SEQ ID NO: 22 - RNA, 7 nt, Homo sapiens
  hsa-miR-193a-3p seed
acuggcc

SEQ ID NO: 23 - RNA, 7 nt, Homo sapiens
  hsa-miR-7-5p seed
ggaagac

SEQ ID NO: 24 - RNA, 7 nt, Homo sapiens
  hsa-miR-323-5p isomiR seed
cugcuug

SEQ ID NO: 25 - RNA, 7 nt, Homo sapiens
  hsa-miR-323-5p isomiR seed
ugcuugg

SEQ ID NO: 26 - RNA, 7 nt, Homo sapiens
  hsa-miR-323-5p isomiR seed
gcugcuu

SEQ ID NO: 27 - RNA, 7 nt, Homo sapiens
  hsa-miR-323-5p isomiR seed
agguggu

SEQ ID NO: 28 - RNA, 7 nt, Homo sapiens
  hsa-miR-323-5p isomiR seed
guggucc

SEQ ID NO: 29 - RNA, 7 nt, Homo sapiens
  hsa-miR-342-5p isomiR seed
gggugcu

SEQ ID NO: 30 - RNA, 7 nt, Homo sapiens
  hsa-miR-342-5p isomiR seed
gcuaucu

SEQ ID NO: 31 - RNA, 7 nt, Homo sapiens
  hsa-miR-342-5p isomiR seed
ggugcua

SEQ ID NO: 32 - RNA, 7 nt, Homo sapiens
  hsa-miR-342-5p isomiR seed
ugugaaa

SEQ ID NO: 33 - RNA, 7 nt, Homo sapiens
  hsa-miR-342-5p isomiR seed
gugcuau

SEQ ID NO: 34 - RNA, 7 nt, Homo sapiens
  hsa-miR-342-5p isomiR seed
ugaaacu

SEQ ID NO: 35 - RNA, 7 nt, Homo sapiens
  hsa-miR-342-5p isomiR seed
cugugaa

SEQ ID NO: 36 - RNA, 7 nt, Homo sapiens
  hsa-miR-342-5p isomiR seed
cuaucug

SEQ ID NO: 37 - RNA, 7 nt, Homo sapiens
  hsa-miR-342-5p isomiR seed
ugcuauc

SEQ ID NO: 38 - RNA, 7 nt, Homo sapiens
  hsa-miR-342-5p isomiR seed
augguua

SEQ ID NO: 39 - RNA, 7 nt, Homo sapiens
  hsa-miR-342-5p isomiR seed
aucugug

SEQ ID NO: 40 - RNA, 7 nt, Homo sapiens
  hsa-miR-342-5p isomiR seed
gaaacug

SEQ ID NO: 41 - RNA, 7 nt, Homo sapiens
  hsa-miR-342-5p isomiR seed
uaucugu

SEQ ID NO: 42 - RNA, 7 nt, Homo sapiens
  hsa-miR-342-5p isomiR seed
gugaaac

SEQ ID NO: 43 - RNA, 7 nt, Homo sapiens
  has-miR-520f-3p  isomiR seed
agugcuu

SEQ ID NO: 44 - RNA, 7 nt, Homo sapiens
  has-miR-520f-3p  isomiR seed
aagugcu

SEQ ID NO: 45 - RNA, 7 nt, Homo sapiens
  hsa-miR-3157-5p isomiR seed
ucagcca

SEQ ID NO: 46 - RNA, 7 nt, Homo sapiens
  hsa-miR-3157-5p isomiR seed
uucagcc

SEQ ID NO: 47 - RNA, 7 nt, Homo sapiens
  hsa-miR-3157-5p isomiR seed
cagccag

SEQ ID NO: 48 - RNA, 7 nt, Homo sapiens
  hsa-miR-3157-5p isomiR seed
uucagcc

SEQ ID NO: 49 - RNA, 7 nt, Homo sapiens
  hsa-miR-193a-3p isomiR seed
acuggcc

SEQ ID NO: 50 - RNA, 7 nt, Homo sapiens
  hsa-miR-7-5p isomiR seed
ggaagac

SEQ ID NO: 51 - RNA, 22 nt, Homo sapiens
  hsa-miR-323-5p mature miRNA
aggugguccguggcgcguucgc

SEQ ID NO: 52 - RNA, 21 nt, Homo sapiens
  hsa-miR-342-5p mature miRNA
aggggugcuaucugugauuga

SEQ ID NO: 53 - RNA, 22 nt, Homo sapiens
  has-miR-520f-3p  mature miRNA
aagugcuuccuuuuagaggguu

SEQ ID NO: 54 - RNA, 23 nt, Homo sapiens
  hsa-mir-520f-3p-i3 mature miRNA
caagugcuuccuuuuagaggguu

SEQ ID NO: 55 - RNA, 22 nt, Homo sapiens
  hsa-miR-3157-5p mature miRNA
uucagccaggcuagugcagucu

SEQ ID NO: 56 - RNA, 22 nt, Homo sapiens
  hsa-miR-193a-3p mature miRNA
aacuggccuacaaagucccagu

SEQ ID NO: 57 - RNA, 23 nt, Homo sapiens
  hsa-miR-7-5p mature miRNA
uggaagacuagugauuuuguugu

SEQ ID NO: 58 - RNA, 20 nt, Homo sapiens
  hsa-miR-323-5p isomiR
aggugguccguggcgcguuc

SEQ ID NO: 59 - RNA, 21 nt, Homo sapiens
  hsa-miR-323-5p isomiR
aggugguccguggcgcguucg

SEQ ID NO: 60 - RNA, 20 nt, Homo sapiens
  hsa-miR-323-5p isomiR
gcugcuugguacuuggagag

SEQ ID NO: 61 - RNA, 19 nt, Homo sapiens
  hsa-miR-323-5p isomiR
aggugguccguggcgcguu

SEQ ID NO: 62 - RNA, 19 nt, Homo sapiens
  hsa-miR-323-5p isomiR
cugcuugguacuuggagag

SEQ ID NO: 63 - RNA, 21 nt, Homo sapiens
  hsa-miR-323-5p isomiR
ugcugcuugguacuuggagag

SEQ ID NO: 64 - RNA, 21 nt, Homo sapiens
  hsa-miR-323-5p isomiR
gaggugguccguggcgcguuc

SEQ ID NO: 65 - RNA, 23 nt, Homo sapiens
  hsa-miR-323-5p isomiR
aggugguccguggcgcguucgcu

SEQ ID NO: 66 - RNA, 18 nt, Homo sapiens
  hsa-miR-323-5p isomiR
ggugguccguggcgcguu

SEQ ID NO: 67 - RNA, 18 nt, Homo sapiens
  hsa-miR-323-5p isomiR
aggugguccguggcgcgu

SEQ ID NO: 68 - RNA, 20 nt, Homo sapiens
  hsa-miR-323-5p isomiR
gaggugguccguggcgcguu

SEQ ID NO: 69 - RNA, 26 nt, Homo sapiens
  hsa-miR-342-5p isomiR
ggggugcuaucugugauugagggaca

SEQ ID NO: 70 - RNA, 25 nt, Homo sapiens
  hsa-miR-342-5p isomiR
ggggugcuaucugugauugagggac

SEQ ID NO: 71 - RNA, 24 nt, Homo sapiens
  hsa-miR-342-5p isomiR
ggggugcuaucugugauugaggga

SEQ ID NO: 72 - RNA, 22 nt, Homo sapiens
  hsa-miR-342-5p isomiR
ggggugcuaucugugauugagg

SEQ ID NO: 73 - RNA, 22 nt, Homo sapiens
  hsa-miR-342-5p isomiR
ugcuaucugugauugagggaca

SEQ ID NO: 74 - RNA, 23 nt, Homo sapiens
  hsa-miR-342-5p isomiR
aggggugcuaucugugauugagg

SEQ ID NO: 75 - RNA, 23 nt, Homo sapiens
  hsa-miR-342-5p isomiR
ggggugcuaucugugauugaggg

SEQ ID NO: 76 - RNA, 27 nt, Homo sapiens
  hsa-miR-342-5p isomiR
aggggugcuaucugugauugagggaca

SEQ ID NO: 77 - RNA, 25 nt, Homo sapiens
  hsa-miR-342-5p isomiR
aggggugcuaucugugauugaggga

SEQ ID NO: 78 - RNA, 20 nt, Homo sapiens
  hsa-miR-342-5p isomiR
ggggugcuaucugugauuga

SEQ ID NO: 79 - RNA, 21 nt, Homo sapiens
  hsa-miR-342-5p isomiR
ugcuaucugugauugagggac

SEQ ID NO: 80 - RNA, 23 nt, Homo sapiens
  hsa-miR-342-5p isomiR
gggugcuaucugugauugaggga

SEQ ID NO: 81 - RNA, 24 nt, Homo sapiens
  hsa-miR-342-5p isomiR
aggggugcuaucugugauugaggg

SEQ ID NO: 82 - RNA, 24 nt, Homo sapiens
  hsa-miR-342-5p isomiR
gggugcuaucugugauugagggac

SEQ ID NO: 83 - RNA, 22 nt, Homo sapiens
  hsa-miR-342-5p isomiR
aggggugcuaucugugauugag

SEQ ID NO: 84 - RNA, 22 nt, Homo sapiens
  hsa-miR-342-5p isomiR
gggugcuaucugugauugaggg

SEQ ID NO: 85 - RNA, 21 nt, Homo sapiens
  hsa-miR-342-5p isomiR
gggugcuaucugugauugagg

SEQ ID NO: 86 - RNA, 20 nt, Homo sapiens
  hsa-miR-342-5p isomiR
ugcuaucugugauugaggga

SEQ ID NO: 87 - RNA, 25 nt, Homo sapiens
  hsa-miR-342-5p isomiR
gggugcuaucugugauugagggaca

SEQ ID NO: 88 - RNA, 26 nt, Homo sapiens
  hsa-miR-342-5p isomiR
aggggugcuaucugugauugagggac

SEQ ID NO: 89 - RNA, 22 nt, Homo sapiens
  hsa-miR-342-5p isomiR
cugugaaacugggcucaaggug

SEQ ID NO: 90 - RNA, 20 nt, Homo sapiens
  hsa-miR-342-5p isomiR
aggggugcuaucugugauug

SEQ ID NO: 91 - RNA, 21 nt, Homo sapiens
  hsa-miR-342-5p isomiR
ggggugcuaucugugauugag

SEQ ID NO: 92 - RNA, 23 nt, Homo sapiens
  hsa-miR-342-5p isomiR
ugcuaucugugauugagggacau

SEQ ID NO: 93 - RNA, 23 nt, Homo sapiens
  hsa-miR-342-5p isomiR
cugugaaacugggcucaagguga

SEQ ID NO: 94 - RNA, 23 nt, Homo sapiens
  hsa-miR-342-5p isomiR
ggugcuaucugugauugagggac

SEQ ID NO: 95 - RNA, 20 nt, Homo sapiens
  hsa-miR-342-5p isomiR
gugaaacugggcucaaggug

SEQ ID NO: 96 - RNA, 20 nt, Homo sapiens
  hsa-miR-342-5p isomiR
gggugcuaucugugauugag

SEQ ID NO: 97 - RNA, 19 nt, Homo sapiens
  hsa-miR-342-5p isomiR
ggggugcuaucugugauug

SEQ ID NO: 98 - RNA, 21 nt, Homo sapiens
  hsa-miR-342-5p isomiR
gcuaucugugauugagggaca

SEQ ID NO: 99 - RNA, 23 nt, Homo sapiens
  hsa-miR-342-5p isomiR
ccugugaaacugggcucaaggug

SEQ ID NO: 100 - RNA, 22 nt, Homo sapiens
  hsa-miR-342-5p isomiR
gugcuaucugugauugagggac

SEQ ID NO: 101 - RNA, 28 nt, Homo sapiens
  hsa-miR-342-5p isomiR
aggggugcuaucugugauugagggacau

SEQ ID NO: 102 - RNA, 19 nt, Homo sapiens
  hsa-miR-342-5p isomiR
ugcuaucugugauugaggg

SEQ ID NO: 103 - RNA, 18 nt, Homo sapiens
  hsa-miR-342-5p isomiR
gggugcuaucugugauug

SEQ ID NO: 104 - RNA, 19 nt, Homo sapiens
  hsa-miR-342-5p isomiR
caugguuaauggaauuguc

SEQ ID NO: 105 - RNA, 27 nt, Homo sapiens
  hsa-miR-342-5p isomiR
ggggugcuaucugugauugagggacau

SEQ ID NO: 106 - RNA, 19 nt, Homo sapiens
  hsa-miR-342-5p isomiR
gggugcuaucugugauuga

SEQ ID NO: 107 - RNA, 19 nt, Homo sapiens
  hsa-miR-342-5p isomiR
uaucugugauugagggaca

SEQ ID NO: 108 - RNA, 21 nt, Homo sapiens
  hsa-miR-342-5p isomiR
gugaaacugggcucaagguga

SEQ ID NO: 109 - RNA, 24 nt, Homo sapiens
  hsa-miR-342-5p isomiR
ccugugaaacugggcucaagguga

SEQ ID NO: 110 - RNA, 20 nt, Homo sapiens
  hsa-miR-342-5p isomiR
ggugcuaucugugauugagg

SEQ ID NO: 111 - RNA, 20 nt, Homo sapiens
  hsa-miR-342-5p isomiR
cuaucugugauugagggaca

SEQ ID NO: 112 - RNA, 19 nt, Homo sapiens
  hsa-miR-342-5p isomiR
ugaaacugggcucaaggug

SEQ ID NO: 113 - RNA, 22 nt, Homo sapiens
  hsa-miR-342-5p isomiR
ugugaaacugggcucaagguga

SEQ ID NO: 114 - RNA, 21 nt, Homo sapiens
  hsa-mir-520f-3p isomiR
aagugcuuccuuuuagagggu

SEQ ID NO: 115 - RNA, 22 nt, Homo sapiens
  hsa-mir-520f-3p isomiR
caagugcuuccuuuuagagggu

SEQ ID NO: 116 - RNA, 21 nt, Homo sapiens
  hsa-miR-3157-5p isomiR
uucagccaggcuagugcaguc

SEQ ID NO: 117 - RNA, 22 nt, Homo sapiens
  hsa-miR-3157-5p isomiR
cuucagccaggcuagugcaguc

SEQ ID NO: 118 - RNA, 21 nt, Homo sapiens
  hsa-miR-3157-5p isomiR
ucagccaggcuagugcagucu

SEQ ID NO: 119 - RNA, 20 nt, Homo sapiens
  hsa-miR-3157-5p isomiR
uucagccaggcuagugcagu

SEQ ID NO: 120 - RNA, 24 nt, Homo sapiens
  hsa-miR-3157-5p isomiR
cuucagccaggcuagugcagucug

SEQ ID NO: 121 - RNA, 20 nt, Homo sapiens
  hsa-miR-193a-3p isomiR
aacuggccuacaaaguccca

SEQ ID NO: 122 - RNA, 21 nt, Homo sapiens
  hsa-miR-193a-3p isomiR
aacuggccuacaaagucccag

SEQ ID NO: 123 - RNA, 24 nt, Homo sapiens
  hsa-miR-7-5p isomiR
uggaagacuagugauuuuguuguu

SEQ ID NO: 124 - RNA, 22 nt, Homo sapiens
  hsa-miR-7-5p isomiR
uggaagacuagugauuuuguug

SEQ ID NO: 125 - RNA, 25 nt, Homo sapiens
  hsa-miR-7-5p isomiR
uggaagacuagugauuuuguuguuc

SEQ ID NO: 126 - RNA, 22 nt, Homo sapiens
  hsa-miR-323-5p mature miRNA sense
gcgaacgcgccacggaccaccu

SEQ ID NO: 127 - RNA, 21 nt, Homo sapiens
  hsa-miR-342-5p mature miRNA sense
ucaaucacagauagcaccccu

SEQ ID NO: 128 - RNA, 22 nt, Homo sapiens
  has-miR-520f-3p  mature miRNA sense
aacccucuaaaaggaagcacuu

SEQ ID NO: 129 - RNA, 23 nt, Homo sapiens
  hsa-mir-520f-3p-i3 mature miRNA sense
aacccucuaaaaggaagcacuug

SEQ ID NO: 130 - RNA, 22 nt, Homo sapiens
  hsa-miR-3157-5p mature miRNA sense
agacugcacuagccuggcugaa

SEQ ID NO: 131 - RNA, 22 nt, Homo sapiens
  hsa-miR-193a-3p mature miRNA sense
acugggacuuuguaggccaguu

SEQ ID NO: 132 - RNA, 23 nt, Homo sapiens
  hsa-miR-7-5p mature miRNA sense
acaacaaaaucacuagucuucca

SEQ ID NO: 133 - RNA, 20 nt, Homo sapiens
  hsa-miR-323-5p isomiR sense
  misc_feature (19)..(20): n is a, c, g, or u
acgcgccacggaccaccunn

SEQ ID NO: 134 - RNA, 21 nt, Homo sapiens
  hsa-miR-323-5p isomiR sense
  misc_feature (20)..(21): n is a, c, g, or u
aacgcgccacggaccaccunn

SEQ ID NO: 135 - RNA, 20 nt, Homo sapiens
  hsa-miR-323-5p isomiR sense
  misc_feature (19)..(20): n is a, c, g, or u
cuccaaguaccaagcagcnn

SEQ ID NO: 136 - RNA, 19 nt, Homo sapiens
  hsa-miR-323-5p isomiR sense
  misc_feature (18)..(19): n is a, c, g, or u
cgcgccacggaccaccunn

SEQ ID NO: 137 - RNA, 19 nt, Homo sapiens
  hsa-miR-323-5p isomiR sense
  misc_feature (18)..(19): n is a, c, g, or u
cuccaaguaccaagcagnn

SEQ ID NO: 138 - RNA, 21 nt, Homo sapiens
  hsa-miR-323-5p isomiR sense
  misc_feature (20)..(21): n is a, c, g, or u
cuccaaguaccaagcagcann

SEQ ID NO: 139 - RNA, 21 nt, Homo sapiens
  hsa-miR-323-5p isomiR sense
  misc_feature (20)..(21): n is a, c, g, or u
acgcgccacggaccaccucnn

SEQ ID NO: 140 - RNA, 23 nt, Homo sapiens
  hsa-miR-323-5p isomiR sense
  misc_feature (22)..(23): n is a, c, g, or u
cgaacgcgccacggaccaccunn

SEQ ID NO: 141 - RNA, 18 nt, Homo sapiens
  hsa-miR-323-5p isomiR sense
  misc_feature (17)..(18): n is a, c, g, or u
cgcgccacggaccaccnn

SEQ ID NO: 142 - RNA, 18 nt, Homo sapiens
  hsa-miR-323-5p isomiR sense
  misc_feature (17)..(18): n is a, c, g, or u
gcgccacggaccaccunn

SEQ ID NO: 143 - RNA, 20 nt, Homo sapiens
  hsa-miR-323-5p isomiR sense
  misc_feature (19)..(20): n is a, c, g, or u
cgcgccacggaccaccucnn

SEQ ID NO: 144 - RNA, 26 nt, Homo sapiens
  hsa-miR-342-5p isomiR sense
  misc_feature (25)..(26): n is a, c, g, or u
ucccucaaucacagauagcaccccnn

SEQ ID NO: 145 - RNA, 25 nt, Homo sapiens
  hsa-miR-342-5p isomiR sense
  misc_feature (24)..(25): n is a, c, g, or u
cccucaaucacagauagcaccccnn

SEQ ID NO: 146 - RNA, 24 nt, Homo sapiens
  hsa-miR-342-5p isomiR sense
  misc_feature (23)..(24): n is a, c, g, or u
ccucaaucacagauagcaccccnn

SEQ ID NO: 147 - RNA, 22 nt, Homo sapiens
  hsa-miR-342-5p isomiR sense
  misc_feature (21)..(22): n is a, c, g, or u
ucaaucacagauagcaccccnn

SEQ ID NO: 148 - RNA, 22 nt, Homo sapiens
  hsa-miR-342-5p isomiR sense
  misc_feature (21)..(22): n is a, c, g, or u
ucccucaaucacagauagcann

SEQ ID NO: 149 - RNA, 23 nt, Homo sapiens
  hsa-miR-342-5p isomiR sense
  misc_feature (22)..(23): n is a, c, g, or u
ucaaucacagauagcaccccunn

SEQ ID NO: 150 - RNA, 23 nt, Homo sapiens
  hsa-miR-342-5p isomiR sense
  misc_feature (22)..(23): n is a, c, g, or u
cucaaucacagauagcaccccnn

SEQ ID NO: 151 - RNA, 27 nt, Homo sapiens
  hsa-miR-342-5p isomiR sense
  misc_feature (26)..(27): n is a, c, g, or u
ucccucaaucacagauagcaccccunn

SEQ ID NO: 152 - RNA, 25 nt, Homo sapiens
  hsa-miR-342-5p isomiR sense
  misc_feature (24)..(25): n is a, c, g, or u
ccucaaucacagauagcaccccunn

SEQ ID NO: 153 - RNA, 20 nt, Homo sapiens
  hsa-miR-342-5p isomiR sense
  misc_feature (19)..(20): n is a, c, g, or u
aaucacagauagcaccccnn

SEQ ID NO: 154 - RNA, 21 nt, Homo sapiens
  hsa-miR-342-5p isomiR sense
  misc_feature (20)..(21): n is a, c, g, or u
cccucaaucacagauagcann

SEQ ID NO: 155 - RNA, 23 nt, Homo sapiens
  hsa-miR-342-5p isomiR sense
  misc_feature (22)..(23): n is a, c, g, or u
ccucaaucacagauagcacccnn

SEQ ID NO: 156 - RNA, 24 nt, Homo sapiens
  hsa-miR-342-5p isomiR sense
  misc_feature (23)..(24): n is a, c, g, or u
cucaaucacagauagcaccccunn

SEQ ID NO: 157 - RNA, 24 nt, Homo sapiens
  hsa-miR-342-5p isomiR sense
  misc_feature (23)..(24): n is a, c, g, or u
cccucaaucacagauagcacccnn

SEQ ID NO: 158 - RNA, 22 nt, Homo sapiens
  hsa-miR-342-5p isomiR sense
  misc_feature (21)..(22): n is a, c, g, or u
caaucacagauagcaccccunn

SEQ ID NO: 159 - RNA, 22 nt, Homo sapiens
  hsa-miR-342-5p isomiR sense
  misc_feature (21)..(22): n is a, c, g, or u
cucaaucacagauagcacccnn

SEQ ID NO: 160 - RNA, 21 nt, Homo sapiens
  hsa-miR-342-5p isomiR sense
  misc_feature (20)..(21): n is a, c, g, or u
ucaaucacagauagcacccnn

SEQ ID NO: 161 - RNA, 20 nt, Homo sapiens
  hsa-miR-342-5p isomiR sense
  misc_feature (19)..(20): n is a, c, g, or u
ccucaaucacagauagcann

SEQ ID NO: 162 - RNA, 25 nt, Homo sapiens
  hsa-miR-342-5p isomiR sense
  misc_feature (24)..(25): n is a, c, g, or u
ucccucaaucacagauagcacccnn

SEQ ID NO: 163 - RNA, 26 nt, Homo sapiens
  hsa-miR-342-5p isomiR sense
  misc_feature (25)..(26): n is a, c, g, or u
cccucaaucacagauagcaccccunn

SEQ ID NO: 164 - RNA, 22 nt, Homo sapiens
  hsa-miR-342-5p isomiR sense
  misc_feature (21)..(22): n is a, c, g, or u
ccuugagcccaguuucacagnn

SEQ ID NO: 165 - RNA, 20 nt, Homo sapiens
  hsa-miR-342-5p isomiR sense
  misc_feature (19)..(20): n is a, c, g, or u
aucacagauagcaccccunn

SEQ ID NO: 166 - RNA, 21 nt, Homo sapiens
  hsa-miR-342-5p isomiR sense
  misc_feature (20)..(21): n is a, c, g, or u
caaucacagauagcaccccnn

SEQ ID NO: 167 - RNA, 23 nt, Homo sapiens
  hsa-miR-342-5p isomiR sense
  misc_feature (22)..(23): n is a, c, g, or u
gucccucaaucacagauagcann

SEQ ID NO: 168 - RNA, 23 nt, Homo sapiens
  hsa-miR-342-5p isomiR sense
  misc_feature (22)..(23): n is a, c, g, or u
accuugagcccaguuucacagnn

SEQ ID NO: 169 - RNA, 23 nt, Homo sapiens
  hsa-miR-342-5p isomiR sense
  misc_feature (22)..(23): n is a, c, g, or u
cccucaaucacagauagcaccnn

SEQ ID NO: 170 - RNA, 20 nt, Homo sapiens
  hsa-miR-342-5p isomiR sense
  misc_feature (19)..(20): n is a, c, g, or u
ccuugagcccaguuucacnn

SEQ ID NO: 171 - RNA, 20 nt, Homo sapiens
  hsa-miR-342-5p isomiR sense
  misc_feature (19)..(20): n is a, c, g, or u
caaucacagauagcacccnn

SEQ ID NO: 172 - RNA, 19 nt, Homo sapiens
  hsa-miR-342-5p isomiR sense
  misc_feature (18)..(19): n is a, c, g, or u
aucacagauagcaccccnn

SEQ ID NO: 173 - RNA, 21 nt, Homo sapiens
  hsa-miR-342-5p isomiR sense
  misc_feature (20)..(21): n is a, c, g, or u
ucccucaaucacagauagcnn

SEQ ID NO: 174 - RNA, 23 nt, Homo sapiens
  hsa-miR-342-5p isomiR sense
  misc_feature (22)..(23): n is a, c, g, or u
ccuugagcccaguuucacaggnn

SEQ ID NO: 175 - RNA, 22 nt, Homo sapiens
  hsa-miR-342-5p isomiR sense
  misc_feature (21)..(22): n is a, c, g, or u
cccucaaucacagauagcacnn

SEQ ID NO: 176 - RNA, 28 nt, Homo sapiens
  hsa-miR-342-5p isomiR sense
  misc_feature (27)..(28): n is a, c, g, or u
gucccucaaucacagauagcaccccunn

SEQ ID NO: 177 - RNA, 19 nt, Homo sapiens
  hsa-miR-342-5p isomiR sense
  misc_feature (18)..(19): n is a, c, g, or u
cucaaucacagauagcann

SEQ ID NO: 178 - RNA, 18 nt, Homo sapiens
  hsa-miR-342-5p isomiR sense
  misc_feature (17)..(18): n is a, c, g, or u
aucacagauagcacccnn

SEQ ID NO: 179 - RNA, 19 nt, Homo sapiens
  hsa-miR-342-5p isomiR sense
  misc_feature (18)..(19): n is a, c, g, or u
caauuccauuaaccaugnn

SEQ ID NO: 180 - RNA, 27 nt, Homo sapiens
  hsa-miR-342-5p isomiR sense
  misc_feature (26)..(27): n is a, c, g, or u
gucccucaaucacagauagcaccccnn

SEQ ID NO: 181 - RNA, 19 nt, Homo sapiens
  hsa-miR-342-5p isomiR sense
  misc_feature (18)..(19): n is a, c, g, or u
aaucacagauagcacccnn

SEQ ID NO: 182 - RNA, 19 nt, Homo sapiens
  hsa-miR-342-5p isomiR sense
  misc_feature (18)..(19): n is a, c, g, or u
ucccucaaucacagauann

SEQ ID NO: 183 - RNA, 21 nt, Homo sapiens
  hsa-miR-342-5p isomiR sense
  misc_feature (20)..(21): n is a, c, g, or u
accuugagcccaguuucacnn

SEQ ID NO: 184 - RNA, 24 nt, Homo sapiens
  hsa-miR-342-5p isomiR sense
  misc_feature (23)..(24): n is a, c, g, or u
accuugagcccaguuucacaggnn

SEQ ID NO: 185 - RNA, 20 nt, Homo sapiens
  hsa-miR-342-5p isomiR sense
  misc_feature (19)..(20): n is a, c, g, or u
ucaaucacagauagcaccnn

SEQ ID NO: 186 - RNA, 20 nt, Homo sapiens
  hsa-miR-342-5p isomiR sense
  misc_feature (19)..(20): n is a, c, g, or u
ucccucaaucacagauagnn

SEQ ID NO: 187 - RNA, 19 nt, Homo sapiens
  hsa-miR-342-5p isomiR sense
  misc_feature (18)..(19): n is a, c, g, or u
ccuugagcccaguuucann

SEQ ID NO: 188 - RNA, 22 nt, Homo sapiens
  hsa-miR-342-5p isomiR sense
  misc_feature (21)..(22): n is a, c, g, or u
accuugagcccaguuucacann

SEQ ID NO: 189 - RNA, 21 nt, Homo sapiens
  hsa-mir-520f-3p isomiR sense
  misc_feature (20)..(21): n is a, c, g, or u
ccucuaaaaggaagcacuunn

SEQ ID NO: 190 - RNA, 23 nt, Homo sapiens
  hsa-mir-520f-3p isomiR sense
  misc_feature (22)..(23): n is a, c, g, or u
cccucuaaaaggaagcacuugnn

SEQ ID NO: 191 - RNA, 21 nt, Homo sapiens
  hsa-miR-3157-5p isomiR sense
  misc_feature (20)..(21): n is a, c, g, or u
cugcacuagccuggcugaann

SEQ ID NO: 192 - RNA, 22 nt, Homo sapiens
  hsa-miR-3157-5p isomiR sense
  misc_feature (21)..(22): n is a, c, g, or u
cugcacuagccuggcugaagnn

SEQ ID NO: 193 - RNA, 21 nt, Homo sapiens
  hsa-miR-3157-5p isomiR sense
  misc_feature (20)..(21): n is a, c, g, or u
acugcacuagccuggcugann

SEQ ID NO: 194 - RNA, 20 nt, Homo sapiens
  hsa-miR-3157-5p isomiR sense
  misc_feature (19)..(20): n is a, c, g, or u
ugcacuagccuggcugaann

SEQ ID NO: 195 - RNA, 24 nt, Homo sapiens
  hsa-miR-3157-5p isomiR sense
  misc_feature (23)..(24): n is a, c, g, or u
gacugcacuagccuggcugaagnn

SEQ ID NO: 196 - RNA, 20 nt, Homo sapiens
  hsa-miR-193a-3p isomiR sense
  misc_feature (19)..(20): n is a, c, g, or u
ggacuuuguaggccaguunn

SEQ ID NO: 197 - RNA, 21 nt, Homo sapiens
  hsa-miR-193a-3p isomiR sense
  misc_feature (20)..(21): n is a, c, g, or u
gggacuuuguaggccaguunn

SEQ ID NO: 198 - RNA, 24 nt, Homo sapiens
  hsa-miR-7-5p isomiR sense
  misc_feature (23)..(24): n is a, c, g, or u
caacaaaaucacuagucuuccann

SEQ ID NO: 199 - RNA, 22 nt, Homo sapiens
  hsa-miR-7-5p isomiR sense
  misc_feature (21)..(22): n is a, c, g, or u
acaaaaucacuagucuuccann

SEQ ID NO: 200 - RNA, 25 nt, Homo sapiens
  hsa-miR-7-5p isomiR sense
  misc_feature (24)..(25): n is a, c, g, or u
acaacaaaaucacuagucuuccann

SEQ ID NO: 201 - RNA, 22 nt, Homo sapiens
  hsa-miR-323-5p mimic sense
gaacgcgccacggaccaccuuu

SEQ ID NO: 202 - RNA, 21 nt, Homo sapiens
  hsa-miR-342-5p mimic sense
aaucacagauagcaccccuuu

SEQ ID NO: 203 - RNA, 20 nt, Homo sapiens
  has-miR-520f-3p  mimic sense
cccucuaaaaggaagcacuu

SEQ ID NO: 204 - RNA, 21 nt, Homo sapiens
  hsa-mir-520f-3p-i3 mimic sense
cccucuaaaaggaagcacuug

SEQ ID NO: 205 - RNA, 22 nt, Homo sapiens
  hsa-miR-3157-5p mimic sense
agacugcacuagccuggcugaa

SEQ ID NO: 206 - RNA, 20 nt, Homo sapiens
  hsa-miR-193a-3p mimic sense
ugggacuuuguaggccaguu

SEQ ID NO: 207 - RNA, 21 nt, Homo sapiens
  hsa-miR-7-5p mimic sense
aacaaaaucacuagucuucca

SEQ ID NO: 208 - RNA, 22 nt, Homo sapiens
  hsa-miR-323-5p mimic sense
  2′-O-methylnucleoside (1)..(2)
gaacgcgccacggaccaccuuu

SEQ ID NO: 209 - RNA, 22 nt, Homo sapiens
  hsa-miR-323-5p mimic antisense
aggugguccguggcgcguucgc

SEQ ID NO: 210 - RNA, 21 nt, Homo sapiens
  hsa-miR-342-5p mimic sense
  2′-O-methylnucleoside (1)..(2)
aaucacagauagcaccccuuu

SEQ ID NO: 211 - RNA, 21 nt, Homo sapiens
  hsa-miR-342-5p mimic antisense
aggggugcuaucugugauuga

SEQ ID NO: 212 - DNA, 22 bp, Homo sapiens
  hsa-miR-520f-3p mimic sense
  RNA (1)..(20)
  2′-O-methylnucleoside (1)..(2)
  2′-O-methylnucleoside (19)..(20)
  DNA (21)..(22)
cccucuaaaaggaagcacuutt

SEQ ID NO: 213 - RNA, 22 nt, Homo sapiens
  hsa-miR-520f-3p mimic antisense
aagugcuuccuuuuagaggguu

SEQ ID NO: 214 - DNA, 23 bp, Homo sapiens
  hsa-miR-520-i3-3p mimic sense
  RNA (1)..(21)
  2′-O-methylnucleoside (1)..(2)
  2′-O-methylnucleoside (20)..(21)
  DNA (22)..(23)
cccucuaaaaggaagcacuugtt

SEQ ID NO: 215 - RNA, 23 nt, Homo sapiens
  hsa-miR-520-i3-3p mimic antisense
caagugcuuccuuuuagaggguu

SEQ ID NO: 216 - RNA, 22 nt, Homo sapiens
  hsa-miR-3157-5p mimic sense
  2′-O-methylnucleoside (1)..(1)
agacugcacuagccuggcugaa

SEQ ID NO: 217 - RNA, 24 nt, Homo sapiens
  hsa-miR-3157-5p mimic antisense
  2′-O-methylnucleoside (22)..(24)
uucagccaggcuagugcagucuua

SEQ ID NO: 218 - DNA, 22 bp, Homo sapiens
  hsa-miR-193a-3p mimic sense
  RNA (1)..(20)
  2′-O-methylnucleoside (1)..(2)
  2′-O-methylnucleoside (19)..(20)
  DNA (21)..(22)
ugggacuuuguaggccaguutt

SEQ ID NO: 219 - RNA, 22 nt, Homo sapiens
  hsa-miR-193a-3p mimic antisense
aacuggccuacaaagucccagu

SEQ ID NO: 220 - DNA, 23 bp, Homo sapiens
  hsa-miR-7-5p mimic sense
  RNA (1)..(21)
  2′-O-methylnucleoside (1)..(2)
  2′-O-methylnucleoside (20)..(21)
  DNA (22)..(23)
aacaaaaucacuagucuuccatt

SEQ ID NO: 221 - RNA, 23 nt, Homo sapiens
  hsa-miR-7-5p mimic antisense
uggaagacuagugauuuuguugu

SEQ ID NO: 222 - DNA, 21 bp, Artificial sequence
  HPRT1 forward primer
tccaaagatggtcaaggtcgc

SEQ ID NO: 223 - DNA, 23 bp, Artificial sequence
  HPRT1 reverse primer
cacgaagatctgcattgtcaagt

SEQ ID NO: 224 - DNA, 18 bp, Artificial sequence
  UBS forward primer
cagccgggatttgggtcg

SEQ ID NO: 225 - DNA, 23 bp, Artificial sequence
  UBS reverse primer
cacgaagatctgcattgtcaagt

SEQ ID NO: 226 - DNA, 20 bp, Artificial sequence
  GUSB forward primer
tgcgtagggacaagaaccac

SEQ ID NO: 227 - DNA, 20 bp, Artificial sequence
  GUSB reverse primer
gggaggggtccaaggatttg

SEQ ID NO: 228 - DNA, 20 bp, Artificial sequence
  mPPIH forward primer
aatcgagctctttgcagacg

SEQ ID NO: 229 - DNA, 20 bp, Artificial sequence
  mPPIH reverse primer
tatcctatcggaacgccatc

SEQ ID NO: 230 - DNA, 20 bp, Artificial sequence
  mSDHA forward primer
gaggaagcacaccctctcat

SEQ ID NO: 231 - DNA, 20 bp, Artificial sequence
  mSDHA reverse primer
ggagcggatagcaggaggta

SEQ ID NO: 232 - DNA, 20 bp, Artificial sequence
  mMCL-1 forward primer
taaggacgaaacgggactgg

SEQ ID NO: 233 - DNA, 20 bp, Artificial sequence
  mMCL-1 reverse primer
cgccttctaggtcctgtacg

SEQ ID NO: 234 - DNA, 20 bp, Artificial sequence
  mENTPD1 forward primer
gccgaatgcatggaactgtc

SEQ ID NO: 235 - DNA, 20 bp, Artificial sequence
  mENTPD1 reverse primer
ctgccgattgttcgctttcc

SEQ ID NO: 236 - DNA, 22 bp, Artificial sequence
  mKRAS forward primer
gtggatgagtatgaccctacga

SEQ ID NO: 237 - DNA, 20 bp, Artificial sequence
  mKRAS reverse primer
ctcctcttgacctgctgtgt

SEQ ID NO: 238 - DNA, 20 bp, Artificial sequence
  mTIM3 forward primer
gcaggatacagttccctggt

SEQ ID NO: 239 - DNA, 20 bp, Artificial sequence
  mTIM3 reverse primer
tctgagctggagtgaccttg

SEQ ID NO: 240 - DNA, 20 bp, Artificial sequence
  hMpp2 fwd primer
ccaggatgatgccaactggt

SEQ ID NO: 241 - DNA, 20 bp, Artificial sequence
  hMpp2 rev primer
atgctttccgcttctcctcc

SEQ ID NO: 242 - DNA, 20 bp, Artificial sequence
  hSTMN1 fwd primer
ccagaattccccctttcccc

SEQ ID NO: 243 - DNA, 20 bp, Artificial sequence
  hSTMN1 rev primer
ccagctgcttcaagacctca

SEQ ID NO: 244 - DNA, 25 bp, Artificial sequence
  hYWHAZ fwd primer
agaaaattgagacggagctaagaga

SEQ ID NO: 245 - DNA, 24 bp, Artificial sequence
  hYWHAZ rev primer
agaagactttgctctctgcttgtg

SEQ ID NO: 246 - DNA, 20 bp, Artificial sequence
  hCCNA2 fwd primer
cggtactgaagtccgggaac

SEQ ID NO: 247 - DNA, 20 bp, Artificial sequence
  hCCNA2 rev primer
tgctttccaaggaggaacgg

SEQ ID NO: 248 - DNA, 20 bp, Artificial sequence
  hNT5E fwd primer
aacaacctgagacacacgga

SEQ ID NO: 249 - DNA, 21 bp, Artificial sequence
  hNT5E rev primer
tggattccattgttgcgttca

SEQ ID NO: 250 - DNA, 22 bp, Artificial sequence
  hENTPD1 fwd primer
gcttcttgtgctatgggaagga

SEQ ID NO: 251 - DNA, 21 bp, Artificial sequenceh
  hENTPD1 rev primer
gatgaaagcatgggtccctga

(19) United States
(12) Patent Application Publication
(10) Pub. No.: US 2021/0038732 A1
(43) Pub. Date: Feb. 11, 2021

(54) ANTICANCER MICRORNA AND LIPID FORMULATIONS THEREOF

(71) Applicant: INTERNA TECHNOLOGIES B.V., Nijmegen (NL)

(72) Inventors: Matheus Maria DE GUNST, Woudenberg (NL); Laurens Adrianus Hendricus VAN PINXTEREN, Den Haag (NL); Michel JANICOT, Brussels (BE); Iman Johannes SCHULTZ, Amersfoort (NL); Roeland Quirinus Jozef SCHAAPVELD, Bussum (NL); Sanaz YAHYANEJAD, Rotterdam (NL)

(73) Assignee: INTERNA TECHNOLOGIES B.V., Nijmegen (NL)

(21) Appl. No.: 16/966,869

(22) PCT Filed: Feb. 12, 2019
(86) PCT No.: PCT/EP2019/053466

§ 371 Date: Jul. 31, 2020

(30) Foreign Application Priority Data
Feb. 12, 2018 (EP) 18156376.8
Apr. 13, 2018 (EP) 18167239.5

(51) Int. Cl. A61K 47/54; A61K 47/69; A61K 31/7105; A61K 31/221; A61K 31/355

(52) U.S. Cl. CPC A61K 47/542; A61K 47/6929; A61K 31/355; A61K 31/221; A61K 31/7105

(57) ABSTRACT

The present invention relates to a lipid formulation comprising microRNA. The formulation comprises cationic lipids that can form lipid nanoparticles with the microRNA. The formulations are useful in medicine.

### FIELD OF THE INVENTION

The present invention relates to a lipid formulation comprising microRNA. The formulation comprises cationic lipids that can form lipid nanoparticles with the microRNA. The formulations are useful in medicine.

### BACKGROUND ART

MicroRNAs (miRNAs) are naturally occurring single-stranded, non-coding small RNA molecules that control gene expression by binding to complementary sequences in their target mRNAs, thereby inhibiting translation or inducing mRNA degradation. miRNAs have recently emerged as key regulators of gene expression during development and are frequently misexpressed in human disease states, in particular cancer. In fact, miRNAs can be used to silence specific cancer genes. Several miRNAs are reported to be effective modulators of cancer. At present, a major challenge in developing miRNA therapies is the lack of an effective delivery system. miRNAs are sensitive to nuclease degradation, and display low physiological stability and may have cytotoxicity in their native form. There is an urgent need for an effective delivery system protecting miRNAs from nuclease degradation, while delivering the functional miRNA molecules or isomiRs or mimics or sources thereof into the cytoplasm of the targeted (cancer) cells without inducing any adverse effects.

Promising delivery systems are those comprising the same materials as cell membranes, or similar lipid or lipid-like materials, allowing the encapsulated miRNA to pass into cells through the cell membrane. Among this class of delivery systems are so-called lipid nanoparticles. Lipid nanoparticles are generally small complex structures, 10-100 nm in diameter, stable in physiological conditions, and immunologically inert (T. Admadzada et al, Biophysical Reviews (2018) 10:69-86). Despite advantages in the delivery of other types of oligonucleotides, there are no known reports of successful miRNA delivery using lipid nanoparticles. There is an ongoing need for effective miRNA nanoparticle formulations to improve the effect of the encapsulated miRNA.

There is an ongoing need for improved microRNA therapies for cancer, as well as an ongoing need for deeper mechanistic insight into microRNA treatment of cancer, which can open up new strategies for treatment. There is an ongoing need for modulation of the cancer immune response.

### DESCRIPTION OF EMBODIMENTS

Surprisingly, the inventors identified a miRNA nanoparticle formulation displaying remarkable in vivo efficacy in several cancer indications.

### Composition

The inventors have surprisingly found that a nanoparticle formulation comprising a diamino lipid provides excellent results. Accordingly, in a first aspect the invention provides a composition comprising a nanoparticle, the nanoparticle comprising a diamino lipid and a miRNA, an antagomiR, or a source thereof, wherein
i) the miRNA or antagomir is a miRNA molecule, an isomiR, or a mimic thereof, and is an anticancer miRNA, preferably an oligonucleotide with a seed sequence comprising at least 6 of the 7 nucleotides of the seed sequence represented by SEQ ID NOs: 17-50, or is an antagomir thereof, and wherein said miRNA or antagomir is preferably selected from the group consisting of miRNA-193a, miRNA-323, miRNA-342, miRNA-520f, miRNA-520f-i3, miRNA-3157, miRNA-7, miRNA-135a, miRNA-135b, and miRNA-196a, or an isomiR thereof, or a mimic thereof, or an antagomir thereof, and wherein
ii) the diamino lipid is of general formula (I)

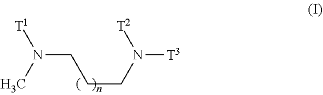

wherein
   n is 0, 1, or 2, and
   T¹, T², and T³ are each independently a C₁₀-C₁₈ chain with optional unsaturations and with zero, one, two, three, or four substitutions, wherein the substitutions are selected from the group consisting of C₁-C₄ alkyl, C₁-C₄ alkenyl, and C₁-C₄ alkoxy.

Such a composition is referred to hereinafter as a composition according to the invention. The nanoparticles comprised in a composition according to the invention are referred to hereinafter as nanoparticles according to the invention. The miRNA or antagomir or source thereof as described under i) are referred to hereinafter as miRNA from the composition; a miRNA from the composition is preferably a miRNA molecule, an isomiR, or a mimic thereof, or a precursor of a miRNA molecule, an isomiR, or a mimic.

In the context of this application, a nanoparticle is a particle with dimensions in the nanometer range, or in some cases in the micrometer range. Preferably, a nanoparticle is as least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200 or more nanometer in diameter, where a diameter is preferably an average diameter of a population of nanoparticles. Preferably, a nanoparticle is at most 100, 150, 200, 250, 300, 350, 400, 450, 500, 550, 600, 650, 700, 750, 800, 850, 900, 950, 1000, 1100, 1200, 1300, 1400, 1500, 2000, 5000, or 10000 nanometer in diameter. More preferably, nanoparticles have an average diameter of 40-300 nm, even more preferably of 50-200 nm, even more preferably of 50-150 nm, most preferably of 65-85 nm, such as about 70 nm.

Nanoparticles according to the invention are lipid nanoparticles that further comprise an oligonucleotide. The oligonucleotide can be seen as the cargo or the payload of the nanoparticle.

Accordingly, the nanoparticles can for example be micelles, liposomes, lipoplexes, unilamellar vesicles, multilamellar vesicles, or cross-linked variants thereof. It is preferred that the nanoparticles are micelles, liposomes, or lipoplexes. When reference is made to the composition of the nanoparticles, reference to the diamino lipid and optional further excipients is intended, and no reference to any cargo substances is intended. As a non-limiting example, when the nanoparticle is said to comprise 50 mol % of the diamino lipid and 50 mol % of other excipients, the molar percentages only relate to the diamino lipid and those other excipients; the oligonucleotide molar fraction or the molar fraction of solvents is not taken into account.

When the invention relates to a composition comprising more than one miRNA molecule, isomiR, mimic, or source thereof or antagomir thereof it is encompassed that each miRNA molecule, isomiR, mimic, or source thereof or antagomir thereof may be present each in a separate composition. Each composition can be sequentially or simultaneously administered to a subject, or mixed prior to use into a single composition. Alternatively, it is also encompassed that more than one miRNA molecules, isomiRs, mimics, or sources thereof or antagomir thereof is present in a composition as defined herein.

Diamino Lipid

The nanoparticle according to the invention comprises a diamino lipid of general formula (I), but it may also comprise further lipids. In preferred embodiments, the diamino lipid is the most prevalent lipid in the nanoparticle by molar percent. As used herein, the term lipid refers to substances that are soluble in nonpolar solvents. The diamino lipids used in the invention have three tails linked to a spacer and thus resemble naturally occurring triglyceride lipids. Several such lipids are known (U.S. Pat. No. 8,691,750).

The diamino lipid of general formula (I) comprises two tertiary amines that are separated by an aliphatic spacer of varying length. The spacer helps determine the headgroup size of the lipid. n can be 0, 1, or 2, so the spacer is in effect an 1,2-ethylene, n-1,3-propylene, or n-1,4-butylene spacer. In particular preferred embodiments, n is 0. In particular preferred embodiments, n is 1. In particular preferred embodiments, n is 2. It is most preferred that n is 1. Accordingly in preferred embodiments the invention provides a composition according to the invention, wherein the diamino lipid is of general formula (I) wherein n is 1. Accordingly, in preferred embodiments the invention provides a composition comprising a nanoparticle, the nanoparticle comprising a diamino lipid and a miRNA, an antagomiR, or a source thereof, wherein
i) the miRNA or antagomir is a miRNA molecule, an isomiR, or a mimic thereof, and is an oligonucleotide with a seed sequence comprising at least 6 of the 7 nucleotides of the seed sequence represented by SEQ ID NOs: 17-50, or is an antagomir thereof, and wherein said miRNA or antagomir is selected from the group consisting of miRNA-193a, miRNA-323, miRNA-342, miRNA-520f, miRNA-520f-i3, miRNA-3157, and miRNA-7, or an isomiR thereof, or a mimic thereof, or an antagomir thereof, and wherein
ii) the diamino lipid is of general formula (I-1)

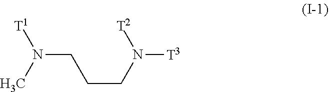

Wherein T¹, T², and T³ are each independently a C₁₀-C₁₈ chain with optional unsaturations and with zero, one, two, three, or four substitutions, wherein the substitutions are selected from the group consisting of C₁-C₄ alkyl, C₁-C₄ alkenyl, and C₁-C₄ alkoxy.

T¹, T², and T³ can be seen as the tails of the lipid, and are aliphatic C₁₀-C₁₈ with optional unsaturations and up to four optional substitutions. T¹, T², and T³ can be independently selected, or the same choice can be made for two or three of T¹, T², and T³. In preferred embodiments, this aspect provides the composition according to the invention, wherein the diamino lipid is of general formula (I) wherein T¹, T², and T³ are identical. Identical should not be so narrowly construed as to imply that the natural abundance of isotopes should be contemplated—identical should preferably only refer to the molecular structure as would be represented in a drawn structural formula.

Longer chains will generally lead to more rigid lipid membranes. In this application the number in C₁₀-C₁₈ refers to the longest continuous chain that can be determined, and not to the total C content. As a non-limiting example, an n-dodecyl chain with an n-propyl substitution at a 6-position comprises 15 C atoms but is a C₁₂ chain because the longest continuous chain has a length of 12 C atoms. Unsaturations can lead to less rigid membranes if the unsaturation is cis in the chain, bending it. A preferred unsaturation is cis. In preferred embodiments, T¹, T², and T³ contain zero, one, two, three, or four unsaturations. In more preferred embodiments, T², and T³ contain one, two, three, or four unsaturations. In even more preferred embodiments, T², and T³ contain one, two, or three unsaturations, preferably three unsaturations.

The optional substitutions are selected from the group consisting of C₁-C₄ alkyl, C₁-C₄ alkenyl, and C₁-C₄ alkoxy A preferred optional substitution is a C₁-C₄ alkyl, more preferably a C₁-C₂ alkyl, most preferably methyl (—CH₃). There are zero, one, two, three, or four of such substitutions, which means that substitutions can be absent. As such the substitutions are optional. Preferably, there are zero, one, two, or three such substitutions.

In preferred embodiments, T¹, T², and T³ are each independently a C₁₀-C₁₆ chain with optional unsaturations and with zero, one, two, three, or four substitutions, wherein the substitutions are selected from the group consisting of C₁-C₄ alkyl, C₁-C₄ alkenyl, and C₁-C₄ alkoxy. In more preferred embodiments, T¹, T², and T³ are each independently a C₁₀-C₁₄ chain with optional unsaturations and with zero, one, two, three, or four substitutions, wherein the substitutions are selected from the group consisting of C₁-C₄ alkyl, C₁-C₄ alkenyl, and C₁-C₄ alkoxy. Most preferably, T¹, T², and T³ are each independently a C₁₂ chain with optional unsaturations and with zero, one, two, three, or four substitutions, wherein the substitutions are selected from the group consisting of C₁-C₄ alkyl, C₁-C₄ alkenyl, and C₁-C₄ alkoxy.

In preferred embodiments, T¹, T², and T³ are each independently a C₁₀-C₁₈ chain with one, two, three, or four unsaturations and with zero, one, two, three, or four substitutions, wherein the substitutions are selected from the group consisting of C₁-C₄ alkyl, C₁-C₄ alkenyl, and C₁-C₄ alkoxy.

In preferred embodiments, T¹, T², and T³ are each independently a C₁₀-C₁₈ chain with one, two, or three unsaturations and with one, two, three, or four substitutions, wherein the substitutions are selected from the group consisting of C₁-C₄ alkyl, C₁-C₄ alkenyl, and C₁-C₄ alkoxy.

In preferred embodiments, T¹, T², and T³ are each independently a C₁₀-C₁₈ chain with one, two, or three unsaturations and with one, two, three, or four substitutions, wherein the substitutions are selected from the group consisting of C₁-C₄ alkyl.

In preferred embodiments, T¹, T², and T³ are each independently a C₁₀-C₁₄ chain with one, two, or three unsaturations and with one, two, or three substitutions, wherein the substitutions are selected from the group consisting of C₁-C₂ alkyl.

Preferred embodiments for T¹, T², and T³ are shown below, with a name for each option appearing below each structural formula. In the systematic Cₙ numbering, a number after a colon (as in C1-C₃) indicates the degree of unsaturation.

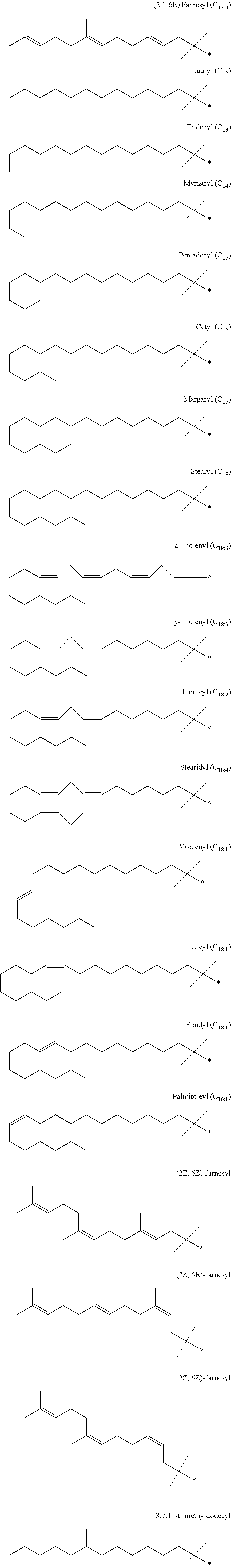

Accordingly, in preferred embodiments this aspect provides the composition according to the invention, wherein the diamino lipid is of general formula (I) wherein T¹, T², and T³ are each independently selected from the group consisting of farnesyl, lauryl, tridecyl, myristryl, pentadecyl, cetyl, margaryl, stearyl, α-linolenyl, γ-linolenyl, linoleyl, stearidyl, vaccenyl, oleyl, elaidyl, palmitoleyl, and 3,7,11-trimethyldodecyl. Preferably, T¹, T², and T³ are each independently selected from the group consisting of farnesyl, lauryl, tridecyl, myristryl, pentadecyl, cetyl, α-linolenyl, γ-linolenyl, linoleyl, stearidyl, oleyl, palmitoleyl, and 3,7,11-trimethyldodecyl. More preferably, T¹, T², and T³ are each independently selected from the group consisting of farnesyl, lauryl, tridecyl, myristryl, stearidyl, palmitoleyl, and 3,7,11-trimethyldodecyl. Even more preferably, T¹, T², and T³ are each independently selected from the group consisting of farnesyl, lauryl, tridecyl, myristryl, and 3,7,11-trimethyldodecyl. Even more preferably, T¹, T², and T³ are each independently selected from the group consisting of farnesyl, lauryl, and 3,7,11-trimethyldodecyl. Most preferably, T¹, T², and T³ are each independently farnesyl, such as (2E,6E) farnesyl, (2E,6Z) farnesyl, (2Z,6E) farnesyl, or (2Z,6Z) farnesyl; preferably they are each (2E,6E) farnesyl.

Farnesyl is also known as 3,7,11-trimethyldodeca-2,6,10-trienyl and is an unsaturated linear C₁₂ chain; it can be (2E,6E), (2E,6Z), (2Z,6E), or (2Z,6Z); preferably it is (2E,6E). Lauryl is also known as dodecyl and is a saturated linear C₁₂ chain. Tridecyl is a saturated linear C₁₃ chain. Myristryl is also known as tetradecyl and is a saturated linear C₁₄ chain. Pentadecyl is a saturated linear C₁₅ chain. Cetyl is also known as palmityl and is a saturated linear C₁₆ chain. Margaryl is also known as heptadecyl and is a saturated linear C₁₇ chain. Stearyl is also known as octadecyl and is a saturated linear C₁₈ chain. α-linolenyl is also known as (9Z,12Z,15Z)-9,12,15-octadecatrienyl and is an unsaturated linear C₁₈ chain. γ-linolenyl is also known as (6Z,9Z, 12Z)-6,9,12-octadecatrienyl and is an unsaturated linear C₁₈ chain. Linoleyl is also known as (9Z,12Z)-9,12-octadecadienyl and is an unsaturated linear C₁₈ chain. Stearidyl is also known as (6Z,9Z,12Z,15Z)-6,9,12,15-octadecatetraenyl and is an unsaturated linear C₁₈ chain. Vaccenyl is also known as (E)-octadec-11-enyl and is an unsaturated linear C₁₈ chain. Oleyl is also known as (9Z)-octadec-9-enyl and is an unsaturated linear C₁₈ chain. Elaidyl is also known as (9E)-octadec-9-enyl and is an unsaturated linear C₁₈ chain. Palmitoleyl is also known as (9Z)-hexadec-9-enyl and is an unsaturated linear C₁₆ chain. 3,7,11-trimethyldodecyl is saturated farnesyl and is a saturated linear C₁₂ chain.

Anticancer miRNA, antagomiR, or a Source Thereof

In preferred embodiments, said anticancer miRNA or antagomir is selected from the group consisting of miRNA-193a, mi RNA-323, mi RNA-342, mi RNA-520f, mi RNA-520f-i3, miRNA-3157, miRNA-135a, miRNA-135b, and miRNA-196a, or an isomiR thereof, or a mimic thereof, or an antagomir thereof. In more preferred embodiments, said miRNA or antagomir is selected from the group consisting of miRNA-193a, miRNA-323, miRNA-342, miRNA-520f, miRNA-520f-i3, miRNA-3157, and miRNA-7, or an isomiR thereof, or a mimic thereof, or an antagomir thereof. In other more preferred embodiments, said miRNA or antagomir is selected from the group consisting of miRNA-193a, miRNA-323, miRNA-342, miRNA-520f, miRNA-520f-i3, and miRNA-3157, or an isomiR thereof, or a mimic thereof, or an antagomir thereof. In other more preferred embodiments, said miRNA or antagomir is a miRNA and is selected from the group consisting of miRNA-193a, miRNA-323, miRNA-342, miRNA-520f, miRNA-520f-i3, and miRNA-3157, or an isomiR thereof, or a mimic thereof, or an antagomir thereof.

Preferred nanoparticles according to the invention comprise a miRNA, an antagomiR, or a source thereof, preferably a miRNA or a source thereof, wherein the miRNA or antagomir is a miRNA molecule, an isomiR, or a mimic thereof, and is an oligonucleotide with a seed sequence comprising at least 6 of the 7 nucleotides of the seed sequence represented by SEQ ID NOs: 17-50, or is an antagomir thereof, and wherein said miRNA or antagomir is selected from the group consisting of miRNA-193a, miRNA-323, miRNA-342, miRNA-520f, miRNA-520f-i3, miRNA-3157, miRNA-7, miRNA-135a, miRNA-135b, and miRNA-196a, or an isomiR thereof, or a mimic thereof, or an antagomir thereof. More preferably, nanoparticles according to the invention comprise a miRNA or a source thereof, wherein the miRNA is a miRNA molecule, an isomiR, or a mimic thereof, and is an oligonucleotide with a seed sequence comprising at least 6 of the 7 nucleotides of the seed sequence represented by SEQ ID NOs: 17-50, and wherein said miRNA is selected from the group consisting of miRNA-193a, miRNA-323, miRNA-342, miRNA-520f, miRNA-520f-i3, miRNA-3157, and miRNA-7, or an isomiR thereof, or a mimic thereof.

MicroRNAs (miRNAs) are small RNAs of 17-25 nucleotides, which function as regulators of gene expression in eukaryotes. miRNAs are initially expressed in the nucleus as part of long primary transcripts called primary miRNAs (pri-miRNAs). Inside the nucleus, pri-miRNAs are partially digested by the enzyme Drosha, to form 65-120 nucleotide-long hairpin precursor miRNAs (pre-miRNAs) that are exported to the cytoplasm for further processing by Dicer into shorter, mature miRNAs, which are the active molecules. In animals, these short RNAs comprise a 5′ proximal “seed” region (generally nucleotides 2 to 8) which appears to be the primary determinant of the pairing specificity of the miRNA to the 3′ untranslated region (3′-UTR) of a target mRNA. A more detailed explanation is given in the part dedicated to general definitions.

Each of the definitions given below concerning a miRNA molecule, a miRNA mimic or a miRNA isomiR or a miRNA antagomir or a source of any of those is to be used for each of the identified miRNAs, molecules or mimics or isomiRs or antagomirs or sources thereof of this application: miRNA miRNA-193a, miRNA-323, miRNA-342, miRNA-520f, miRNA-520f-i3, miRNA-3157, and miRNA-7, or isomiRs or mimics or antagomirs or sources thereof. Preferred mature sequences (SEQ ID NOs: 51-57), seed sequences (SEQ ID NOs: 17-50, where SEQ ID NOs: 17-23 are seed sequences for canonical miRNAs and SEQ ID NOs: 24-50 are seed sequences for isomiRs), isomiR sequences (SEQ ID NOs: 58-125), or source sequences (RNA precursor as SEQ ID NOs: 1-8, or DNA encoding a RNA precursor as SEQ ID NOs: 9-16) of said miRNA molecule or mimic or isomiR thereof respectively are identified in the sequence listing.

In preferred embodiments, this aspect provides the composition according to the invention, wherein said miRNA is
i) a miRNA-323-5p molecule, a miRNA-323-5p isomiR, or a miRNA-323-5p mimic, or
ii) a miRNA-342-5p molecule, a miRNA-324-5p isomiR, or a miRNA-324-5p mimic, or
iii) a miRNA-520f-3p molecule, a miRNA-520f-3p isomiR, or a miRNA-520f-3p mimic, or
iv) a miRNA-520f-3p-i3 molecule, a miRNA-520f-3p-i3 isomiR, or a miRNA-520f-3p-i3 mimic, or
v) a miRNA-3157-5p molecule, a miRNA-3157-5p isomiR, or a miRNA-3157-5p mimic, or
vi) a miRNA-193a-3p molecule, a miRNA-193a-3p isomiR, or a miRNA-193a-3p mimic, or
vii) a miRNA-7-5p molecule, a miRNA-7-5p isomiR, or a miRNA-7-5p mimic.

In other preferred embodiments, this aspect provides the composition according to the invention, wherein said miRNA or antagomir is a miRNA-135a molecule, a miRNA-135b molecule, a miRNA-196a-5p molecule, an isomiR of miRNA-135a, an isomiR of miRNA-135b, an isomiR of miRNA-196a-5p, an antagomir of miRNA-135a, an antagomir of miRNA-135b, an antagomir of miRNA-196a-5p, or a mimic thereof.

A mimic is a molecule which has a similar or identical activity with a miRNA molecule. In this context a similar activity is given the same meaning as an acceptable level of an activity. A mimic is, in a functional determination, opposed to an antagomir. Preferred mimics are synthetic oligonucleotides, preferably comprising one or more nucleotide analogues such as locked nucleic acid monomers, and/or nucleotides comprising scaffold modifications and/or nucleotides comprising base modifications. A mimic can be a mimic for a miRNA or for an isomiR, and it can also be a mimic for an antagomir. Preferred mimics are mimics for a miRNA or for an isomiR.

Preferred mimics are double stranded oligonucleotides comprising a sense strand and an antisense strand. The canonical miRNA as it naturally occurs is defined herein as having an antisense sequence, because it is complementary to the sense sequence of naturally occurring targets. It follows that in a double stranded mimic as is a preferred mimic for the composition according to the invention, there are two strands, one of which is designated as a sense strand, and one of which is designated as an antisense strand. The antisense strand can have the same sequence as a miRNA, or as a precursor of a miRNA, or as an isomiR, or it can have the same sequence as a fragment thereof, or comprise the same sequence, or comprise the same sequence as a fragment thereof. The sense strand is at least partially reverse complementary to the antisense strand, to allow formation of the double stranded mimic. The sense strand is not necessarily biologically active per se, one of its important functions is to stabilize the antisense strand or to prevent its degradation. Examples of sense strands for mature miRNAs are SEQ ID NOs 126-132. Examples of sense strands for isomiRs are SEQ ID NOs: 133-200.

In preferred embodiments an antisense strand comprises at least one modified nucleoside, preferably selected from the group consisting of a bridged nucleic acid nucleoside such as a locked nucleic acid (LNA) nucleoside, a 2′-O-alkylnucleoside such as a 2′-O-methylnucleoside, a 2′-fluoronucleoside, and a 2′-azidonucleoside, preferably a 2′-O-alkylnucleoside such as a 2′-O-methylnucleoside. It is preferred that such an at least one modified nucleoside replaces the first or the last RNA nucleoside, or replaces the second or second-to-last RNA nucleoside. In preferred embodiments at least two modified nucleosides replace the first two or the last two RNA nucleosides.

More preferably both the first and the last RNA nucleosides are replaced, even more preferably both the first two and the last two. It is to be understood that the replacing modified nucleoside has the same pairing capacity as the nucleoside it replaces, preferably it has the same nucleobase. Preferably an antisense strand does not comprise modified nucleosides outside of the first two or the last two RNA nucleosides. In preferred embodiments, the last base of an antisense strand is a DNA nucleoside; more preferably the last two bases of an antisense strand are DNA nucleosides. Preferably the last one or two residues of an antisense strand form an overhang when the antisense strand forms a pair with the sense strand; more preferably the last two residues of an antisense strand form such an overhang. Preferably an antisense sense does not comprise DNA nucleosides outside of the last two nucleosides, or outside of an overhang. Preferably a sense strand comprises only RNA nucleosides.

Preferably, the sense strand and the antisense strand do not fully overlap, having one, two, three, or four additional bases at their 3′-end, preferably having two additional bases at their 3′-end, forming a sticky end. Accordingly, in the corresponding antisense strand, the 3′-end one, two, three, or four bases preferably do not have a reverse complementary base in the sense strand, also forming a sticky end; more preferably the first two bases of a sense strand form a sticky end, not having complementary bases in the antisense strand. The sense strand is not necessarily biologically active, it serves primarily to increase the stability of the antisense strand. Examples of preferred sequences for sense/antisense pairs for mimics are SEQ ID NOs: 201-207, 208, 210, 212, 214, 216, 218, and 220 for sense strands, more preferably SEQ ID NOs: 208, 210, 212, 214, 216, 218, and 220 for sense strands, and SEQ ID NOs: 209, 211, 213, 215, 217, 219, and 221 for antisense strands. Preferred pairs are SEQ ID NOs: 201 or 208 and SEQ ID NO: 209, SEQ ID NOs: 202 or 210 and SEQ ID NO: 211, SEQ ID NOs: 203 or 212 and SEQ ID NO: 213, SEQ ID NOs: 204 or 214 and SEQ ID NO: 215, SEQ ID NOs: 205 or 216 and SEQ ID NO: 217, SEQ ID NOs: 206 or 218 and SEQ ID NO: 219, and SEQ ID NOs: 207 or 220 and SEQ ID NO: 221, more preferably SEQ ID NO: 218 and SEQ ID NO: 219.

In preferred embodiments, a mimic is a double stranded oligonucleotide comprising a sense strand and an antisense strand, wherein both strands have a length of 15 to 30 nucleotides, preferably of 17 to 27 nucleotides, wherein the antisense strand has 70, 75, 80, 85, 90, 95, 96, 97, 98, 99, or 100% sequence identity with any one of SEQ ID NOs: 51-125, wherein the sense strand optionally has 70, 75, 80, 85, 90, 95, 96, 97, 98, 99, or 100% sequence identity with any one of SEQ ID NOs: 126-200, wherein the sense strand and the antisense strand preferably can anneal to form said double stranded oligonucleotide, wherein optionally one or both ends of the oligonucleotide are sticky ends having an overlap of one, two, three, or four, preferably of two nucleotides, wherein the sense strand optionally comprises chemically modified nucleotides. Preferably, the two strands of a double stranded mimic have the same length, or differ by one, two, three, four, five, or six nucleotides in length.

An antagomir of a miRNA molecule, isomiR, mimic, or source thereof is a molecule which has an activity which is opposite or reverse to the one of the corresponding miRNA molecule it derives from. An antagomir of a miRNA, isomiR, or mimic may also be defined as a molecule which is able to antagonize or silence or decrease an activity of said miRNA molecule or isomiR or mimic. An activity which is opposite or reverse to the one of the corresponding miRNA molecule it derives from or an activity which is able to antagonize an activity of said miRNA molecule it derives from is preferably an activity which is able to decrease an activity of said miRNA molecule or isomiR or mimic or source thereof. In this context, decrease means at least 5%, 10%, 20%, 30%, 40%, 50%, 60%, 70%, 80%, 90%, 95% or 100% decrease of the activity of said miRNA molecule or isomiR or mimic or source thereof. A mimic of an antagomir can be a synthetic oligonucleotide that has chemical modifications such as later defined herein. Preferred activities and preferred assays for assessing said activities are later on defined herein.

Within the whole text of the application unless otherwise indicated, a miRNA may also be named a miRNA molecule, a miR, an isomiR, an antagomir, or a mimic, or a source or a precursor thereof. Each sequence identified herein may be identified as being SEQ ID NO as used in the text of the application or as corresponding SEQ ID NO in the sequence listing. A SEQ ID NO as identified in this application may refer to the base sequence of said miRNA, isomiR, antagomir, mimic, or source thereof such as a precursor. For all SEQ ID NOs, a skilled person knows that some bases can be interchanged. For example, each instance of T can be individually substituted by U, and vice versa. An RNA sequence provided for a mature miRNA can for example be synthesized as a DNA oligonucleotide using DNA nucleotides instead of RNA nucleotides. In such a case, thymine bases can be used instead of uracil bases. Alternately, thymine bases on deoxyribose scaffolds can be used. A skilled person understands that the base pairing behaviour is more important than the exact sequence, and that T and U are generally interchangeable for such purposes. Accordingly, an antagomir can be either a DNA or an RNA molecule, or a further modified oligonucleotide as defined later herein. Accordingly, a mimic can be either a DNA or an RNA molecule, or a further modified oligonucleotide as defined later herein.

MiRNA antagomirs are also referred to in the present invention. This term relates to miRNA molecules of this invention whose expression is not to be up-regulated/over-expressed/increased and/or whose activity is not to be increased in order to be used in therapeutic applications as identified herein. In contrast, the endogenous expression of these miRNA molecules needs to be down-regulated/decreased and/or an activity of such miRNA molecule needs to be decreased or reduced or inhibited to obtain a therapeutically desirable effect. This is preferably carried out as explained later herein using an antagomir. Therefore, in the invention when reference is made to any of these miRNA molecules in a therapeutic use, one always refers to a use of an antagomir of a miRNA-135a, miRNA-135b, or miRNA-196a-5p molecule or of a mimic of an antagomir of these miRNAs or a source of an antagomir of these miRNAs. Accordingly, when one refers to an antagomir, one always refers to a use of an antagomir of a miRNA-135a, miRNA-135b, or miRNA-196a-5p molecule or a mimic or a source thereof as indicated herein. Each of the definitions given herein concerning a miRNA molecule or a mimic or an isomiR or a source of any of those may also apply for any of the miRNA molecules to be used as an antagomir as identified in this paragraph. Each definition given herein concerning a given antagomir of a miRNA molecule also holds for other antagomir of a distinct miRNA molecule, each as defined herein. An antagomir is preferably complementary or reverse complementary to a miRNA, isomiR, or mimic thereof.

In the context of the invention, a miRNA molecule or a mimic or an isomiR or an antagomir thereof may be a synthetic or natural or recombinant or mature or part of a mature miRNA or a human miRNA or derived from a human miRNA as further defined in the part dedicated to the general definitions. A human miRNA molecule is a miRNA molecule which is found in a human cell, tissue, organ or body fluids (i.e. endogenous human miRNA molecule). A human miRNA molecule may also be a human miRNA molecule derived from an endogenous human miRNA molecule by substitution, deletion and/or addition of a nucleotide. A miRNA molecule or a mimic or an isomiR or an antagomir thereof may be a single stranded or double stranded RNA molecule.

Preferably a miRNA molecule or a mimic or an isomiR thereof is from 6 to 30 nucleotides in length, preferably 12 to 30 nucleotides in length, preferably 15 to 28 nucleotides in length, more preferably said molecule has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

Preferably an antagomir of a miRNA molecule is from 8 to 30 nucleotides in length, preferably 10 to 30 nucleotides in length, preferably 12 to 28 nucleotides in length, more preferably said molecule has a length of at least 8, 9, 10, 11, 12, 13, 14, 15, 16, 17, 18, 19, 20, 21, 22, 23, 24, 25, 26, 27, 28, 29, 30 nucleotides or more.

In a preferred embodiment, a miRNA molecule or a mimic or isomiR comprises at least 6 of the 7 nucleotides present in the seed sequence of said miRNA molecule or a mimic or isomiR thereof (SEQ ID NOs: 17-50), or is an antagomir thereof. Preferably in this embodiment, a miRNA molecule or a mimic or isomiR is from 6 to 30 nucleotides in length and more preferably comprises at least 6 of the 7 nucleotides present in the seed sequence of said miRNA molecule or mimic or isomiR, or is an antagomir thereof of the same length. Even more preferably a miRNA molecule or a mimic or isomiR is from 15 to 28 nucleotides in length and more preferably comprises at least 6 of the 7 nucleotides present in the seed sequence, even more preferably a miRNA molecule has a length of at least 6, 7, 8, 9, 10, 11, 12, 13, 14, 15, 16, 17, 18, 19, 20, 21, 22, 23, 24, 25, 26, 27, 28, 29, 30 nucleotides or more, or is an antagomir thereof of the same length.

In this context, to comprise at least 6 of the 7 nucleotides present in a seed sequence is intended to refer to a continuous stretch of 7 nucleotides that differs from the seed sequence in at most one position. Alternately, this can refer to a continuous stretch of 6 nucleotides that differs from the seed sequence only through omission of a single nucleotide. Throughout the application, more preferred miRNA molecules, isomiRs, mimics, or precursors thereof comprise all 7 of the 7 nucleotides present in an indicated seed sequence, or in other words have 100% sequence identity with said seed sequences. Preferably, when comprised in a miRNA, isomiR, or mimic, a seed sequence starts at nucleotide number 1, 2, or 3, and ends at nucleotide number 7, 8, 9, 10, or 11; most preferably such a seed sequence starts at nucleotide number 2 and ends at nucleotide number 8.

Preferred miRNA-135a, miRNA-135b, and miRNA-196a molecules, isomiRs, or mimics thereof are described in EP17199997, in tables 2, 4, 5, and 6. Preferred precursors thereof are described in tables 1 and 3 of EP17199997. Preferred miRNA-135a, miRNA-135b, and miRNA-196a molecules, isomiRs, or mimics thereof comprise at least 6 of the 7 nucleotides present in the seed sequences identified in tables 4 or 5 of EP17199997 and more preferably have a length of at least 6, 7, 8, 9, 10, 11, 12, 13, 14, 15, 16, 17, 18, 19, 20, 21, 22, 23, 24, 25, 26, 27, 28, 29, 30 nucleotides or more. Preferably, for an antagomir, a sequence reverse complementary to at least 6 of the 7 nucleotides present in the seed sequence identified in tables 4 or 5 of EP17199997 is comprised instead. A preferred antagomir of miRNA-135a, miRNA-135b, or miRNA-196a is complementary or reverse complementary to the miRNA-135a, miRNA-135b, or miRNA-196a molecule, isomiR, or mimic thereof as described above, and is preferably as described in table 6 of EP17199997.

A preferred miRNA-323 is a miRNA-323-5p molecule, isomiR, or mimic thereof and comprises at least 6 of the 7 nucleotides present in the seed sequence of SEQ ID NOs: 17 or 24-28 and more preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more. Preferably, for an antagomir, a sequence reverse complementary to at least 6 of the 7 nucleotides present in the seed sequence of SEQ ID NOs: 17 or 24-28 is comprised instead. A preferred antagomir of miRNA-323 is complementary or reverse complementary to the miRNA-323 molecule, isomiR, or mimic thereof as described above.

A preferred mimic of miRNA-323 has a sense strand and an antisense strand, wherein the antisense strand comprises at least 6 of the 7 nucleotides present in the seed sequence of SEQ ID NOs: 17 or 24-28 and wherein the antisense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more, and wherein the antisense strand preferably has at least 70%, 75%, 80%, 85%, 90%, 95%, 97%, 98%, 99% or 100% identity over SEQ ID NOs: 51, 58-68, or 209 and wherein the sense strand preferably has at least 70%, 75%, 80%, 85%, 90%, 95%, 97%, 98%, 99% or 100% identity over SEQ ID NOs: 126, 133-143, 201, or 208 and wherein the sense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

A preferred miRNA-342 is a miRNA-342-5p molecule, isomiR, or mimic thereof and comprises at least 6 of the 7 nucleotides present in the seed sequence of SEQ ID NOs: 18 or 29-42 and more preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more. Preferably, for an antagomir, a sequence reverse complementary to at least 6 of the 7 nucleotides present in the seed sequence of SEQ ID NOs: 18 or 29-42 is comprised instead. A preferred antagomir of miRNA-342 is complementary or reverse complementary to the miRNA-342 molecule, isomiR, or mimic thereof as described above.

A preferred mimic of miRNA-342 has a sense strand and an antisense strand, wherein the antisense strand comprises at least 6 of the 7 nucleotides present in the seed sequence of SEQ ID NOs: 18 or 29-42 and wherein the antisense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more, and wherein the antisense strand preferably has at least 70%, 75%, 80%, 85%, 90%, 95%, 97%, 98%, 99% or 100% identity over SEQ ID NOs: 52, 69-113, or 211 and wherein the sense strand preferably has at least 70%, 75%, 80%, 85%, 90%, 95%, 97%, 98%, 99% or 100% identity over SEQ ID NOs: 127, 144-188, 202, or 210 and wherein the sense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

A preferred miRNA-520f is a miRNA-520f-3p molecule, isomiR, or mimic thereof and comprises at least 6 of the 7 nucleotides present in the seed sequence of SEQ ID NOs: 19 or 43-44 and more preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more. Preferably, for an antagomir, a sequence reverse complementary to at least 6 of the 7 nucleotides present in the seed sequence of SEQ ID NOs: 19 or 43-44 is comprised instead. A preferred antagomir of miRNA-520f is complementary or reverse complementary to the miRNA-520f molecule, isomiR, or mimic thereof as described above.

A preferred mimic of miRNA-520f has a sense strand and an antisense strand, wherein the antisense strand comprises at least 6 of the 7 nucleotides present in the seed sequence of SEQ ID NOs: 19 or 43-44 and wherein the antisense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more, and wherein the antisense strand preferably has at least 70%, 75%, 80%, 85%, 90%, 95%, 97%, 98%, 99% or 100% identity over SEQ ID NOs: 53, 114, 115, or 213 and wherein the sense strand preferably has at least 70%, 75%, 80%, 85%, 90%, 95%, 97%, 98%, 99% or 100% identity over SEQ ID NOs: 128, 189, 190, 203, or 212, and wherein the sense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

A further preferred miRNA-520f is a miRNA-520f-3p-i3 molecule or mimic thereof comprises at least 6 of the 7 nucleotides present in the seed sequence of SEQ ID NO: 20 and more preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more. Preferably, for an antagomir, a sequence reverse complementary to at least 6 of the 7 nucleotides present in the seed sequence of SEQ ID NO: 20 is comprised instead. A preferred antagomir of miRNA-520f-3p-i3 is complementary or reverse complementary to the miRNA-520f-3p-i3 molecule or mimic thereof as described above.

A preferred mimic of miRNA-520f-3p-i3 has a sense strand and an antisense strand, wherein the antisense strand comprises at least 6 of the 7 nucleotides present in the seed sequence of SEQ ID NO: 20 and wherein the antisense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more, and wherein the antisense strand preferably has at least 70%, 75%, 80%, 85%, 90%, 95%, 97%, 98%, 99% or 100% identity over SEQ ID NOs: 54 or 215, and wherein the sense strand preferably has at least 70%, 75%, 80%, 85%, 90%, 95%, 97%, 98%, 99% or 100% identity over SEQ ID NOs: 129, 204, or 214 and wherein the sense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

A preferred miRNA-3157 is a miRNA-3157-5p molecule, isomiR, or mimic thereof and comprises at least 6 of the 7 nucleotides present in the seed sequence of SEQ ID NOs: 21 or 45-48 and more preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more. Preferably, for an antagomir, a sequence reverse complementary to at least 6 of the 7 nucleotides present in the seed sequence of SEQ ID NOs: 21 or 45-48 is comprised instead. A preferred antagomir of miRNA-3157 is complementary or reverse complementary to the miRNA-3157 molecule, isomiR, or mimic thereof as described above.

A preferred mimic of miRNA-3157 has a sense strand and an antisense strand, wherein the antisense strand comprises at least 6 of the 7 nucleotides present in the seed sequence of SEQ ID NOs: 21 or 45-48 and wherein the antisense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more, and wherein the antisense strand preferably has at least 70%, 75%, 80%, 85%, 90%, 95%, 97%, 98%, 99% or 100% identity over SEQ ID NOs: 55, 116-120, or 217, and wherein the sense strand preferably has at least 70%, 75%, 80%, 85%, 90%, 95%, 97%, 98%, 99% or 100% identity over SEQ ID NOs: 130, 191-195, 205, or 216, and wherein the sense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

A preferred miRNA-193a is a miRNA-193a-3p, more preferably a miRNA-193a-3p molecule, isomiR, or mimic thereof, and comprises at least 6 of the 7 nucleotides present in the seed sequence of SEQ ID NOs: 22 or 49 and more preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more. Preferably, for an antagomir, a sequence reverse complementary to at least 6 of the 7 nucleotides present in the seed sequence of SEQ ID NOs: 22 or 49 is comprised instead. A preferred antagomir of miRNA-193a is complementary or reverse complementary to the miRNA-193a molecule, isomiR, or mimic thereof as described above.

A preferred mimic of miRNA-193a has a sense strand and an antisense strand, wherein the antisense strand comprises at least 6 of the 7 nucleotides present in the seed sequence of SEQ ID NOs: 22 or 49 and wherein the antisense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more, and wherein the antisense strand preferably has at least 70%, 75%, 80%, 85%, 90%, 95%, 97%, 98%, 99% or 100% identity over SEQ ID NOs: 56, 121, 122, or 219, preferably 56 or 219, more preferably 219, and wherein the sense strand preferably has at least 70%, 75%, 80%, 85%, 90%, 95%, 97%, 98%, 99% or 100% identity over SEQ ID NOs: 131, 196, 197, 206, or 218, more preferably 218, and wherein the sense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

A preferred miRNA-7 is a miRNA-7-5p molecule, isomiR, or mimic thereof and comprises at least 6 of the 7 nucleotides present in the seed sequence of SEQ ID NOs: 23 or 50 and more preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more. Preferably, for an antagomir, a sequence reverse complementary to at least 6 of the 7 nucleotides present in the seed sequence of SEQ ID NOs: 23 or 50 is comprised instead. A preferred antagomir of miRNA-7 is complementary or reverse complementary to the miRNA-7 molecule, isomiR, or mimic thereof as described above.

A preferred mimic of miRNA-7 has a sense strand and an antisense strand, wherein the antisense strand comprises at least 6 of the 7 nucleotides present in the seed sequence of SEQ ID NOs: 23 or 50 and wherein the antisense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more, and wherein the antisense strand preferably has at least 70%, 75%, 80%, 85%, 90%, 95%, 97%, 98%, 99% or 100% identity over SEQ ID NOs: 57, 123-125, or 221, and wherein the sense strand preferably has at least 70%, 75%, 80%, 85%, 90%, 95%, 97%, 98%, 99% or 100% identity over SEQ ID NOs: 132, 198-200, 207, or 220, and wherein the sense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

Preferably, a miRNA molecule, isomiR, or mimic thereof has a length of at least 6, 7, 8, 9, 10, 11, 12, 13, 14, 15, 16, 17, 18, 19, 20, 21, 22, 23, 24, 25, 26, 27, 28, 29, 30 nucleotides or more, comprises at least 6 of the 7 nucleotides present in a given seed sequence of any one of SEQ ID NOs: 17-50 and has at least 70% identity over the whole mature sequence of any one of SEQ ID NOs: 51-125. Preferably, identity is at least 75%, 80%, 85%, 90%, 95%, 97%, 98%, 99% or 100%.

Alternatively, preferably, a miRNA molecule, isomiR, or mimic thereof has a length of not more than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides, comprises at least 6 of the 7 nucleotides present in a given seed sequence of any one of SEQ ID NOs: 17-50 and has at least 70% identity over the whole mature sequence of any one of SEQ ID NOs: 51-125. Preferably, identity is at least 75%, 80%, 85%, 90%, 95%, 97%, 98%, 99% or 100%.

In another preferred embodiment, an isomiR of a miRNA molecule has at least 70% identity over the whole isomiR sequence of any one of SEQ ID NOs: 58-125. Preferably, identity is at least 75%, 80%, 85%, 90%, 95% or higher. Preferably in this embodiment, an isomiR of a miRNA molecule or a mimic thereof has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

Accordingly a preferred miRNA-323 molecule, isomiR, or mimic thereof is a miRNA-323-5p molecule, isomiR, or mimic thereof and comprises at least 6 of the 7 nucleotides present in the seed sequence identified as SEQ ID NOs: 17, 24-28 and/or has at least 70%, 75%, 80%, 85%, 90%, 95%, 97%, 98%, 99% or 100% identity over SEQ ID NOs: 51, 58-68 and/or has a length of at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides or more.

Accordingly a preferred miRNA-323 molecule, isomiR, or mimic thereof is a miRNA-323-5p molecule, isomiR, or mimic thereof and comprises at least 6 of the 7 nucleotides present in the seed sequence identified as SEQ ID NOs: 17, 24-28 and/or has at least 70%, 75%, 80%, 85%, 90%, 95%, 97%, 98%, 99% or 100% identity over SEQ ID NOs: 51, 58-68 and/or has a length of at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides or more.

Accordingly a preferred miRNA-342 molecule, isomiR, or mimic thereof is a miRNA-342-5p molecule, isomiR, or mimic thereof and comprises at least 6 of the 7 nucleotides present in the seed sequence identified as SEQ ID NOs: 18, 29-42 and/or has at least 70%, 75%, 80%, 85%, 90%, 95%, 97%, 98%, 99% or 100% identity over SEQ ID NOs: 52, 69-113 and/or has a length of at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides or more.

Accordingly a preferred miRNA-520f molecule, isomiR, or mimic thereof is a miRNA-520f-3p molecule, isomiR, or mimic thereof and comprises at least 6 of the 7 nucleotides present in the seed sequence identified as SEQ ID NOs: 19, 43-44 and/or has at least 70%, 75%, 80%, 85%, 90%, 95%, 97%, 98%, 99% or 100% identity over SEQ ID NOs: 53, 114-115 and/or has a length of at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides or more. A further preferred miRNA 520f molecule, isomiR, or mimic thereof is a miRNA-520f-3p-i3 molecule or a mimic thereof and comprises at least 6 of the 7 nucleotides present in the seed sequence identified as SEQ ID NO: 20 and/or has at least 70%, 75%, 80%, 85%, 90%, 95%, 97%, 98%, 99% or 100% identity over SEQ ID NO: 54 and/or has a length of at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides or more.

Accordingly a preferred miRNA-3157 molecule, isomiR, or mimic thereof is a miRNA-3157-5p molecule, isomiR, or mimic thereof and comprises at least 6 of the 7 nucleotides present in the seed sequence identified as SEQ ID NOs: 21, 45-48 and/or has at least 70%, 75%, 80%, 85%, 90%, 95%, 97%, 98%, 99% or 100% identity over SEQ ID NOs: 55, 116-120 and/or has a length of at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides or more.

Accordingly a preferred miRNA-193a molecule, isomiR, or mimic thereof is a miRNA-193a-3p molecule, isomiR, or mimic thereof and comprises at least 6 of the 7 nucleotides present in the seed sequence identified as SEQ ID NOs: 22 or 49 and/or has at least 70%, 75%, 80%, 85%, 90%, 95%, 97%, 98%, 99% or 100% identity over SEQ ID NOs: 56, 121-122 and/or has a length of at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides or more.

Accordingly a preferred miRNA-7 molecule, isomiR, or mimic thereof is a miRNA-7-5p molecule, isomiR, or mimic thereof and comprises at least 6 of the 7 nucleotides present in the seed sequence identified as SEQ ID NOs: 23 or 50 and/or has at least 70%, 75%, 80%, 85%, 90%, 95%, 97%, 98%, 99% or 100% identity over SEQ ID NOs: 57, 123-125 and/or has a length of at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides or more.

Another preferred miRNA molecule, isomiR, or mimic thereof has at least 60% identity with a seed sequence of any one of SEQ ID NOs: 17-50, or with a mature sequence of any one of SEQ ID NOs: 51-57, or with a precursor sequence of any one of SEQ ID NOs: 1-16, preferably of any one of SEQ ID NOs: 1-8, or with a DNA encoding an RNA precursor of any one of SEQ ID NOs: 9-16, or with an isomiR sequence of any one of SEQ ID NOs: 58-125. Identity may be at least 65%, 70%, 75%, 80%, 85%, 90%, 95%, 99% or 100%. Identity is preferably assessed on the whole SEQ ID NO as identified in a given SEQ ID NO. However, identity may also be assessed on part of a given SEQ ID NO. Part may mean at least 50% of the length of the SEQ ID NO, at least 60%, 70%, 80%, 90% or 100%.

A precursor sequence may result in more than one isomiR sequences depending on the maturation process—see for example miRNA-323 (mature sequence SEQ ID NO: 51) where in certain tissues multiple isomiRs have been identified (SEQ ID NOs: 58-68). IsomiRs of a miRNA molecule stem from the same precursor, and conversely a precursor can lead to multiple miRNA molecules, one of which is referred to as the canonical miRNA (such as miRNA-323-5p, SEQ ID NO: 51) and others being referred to as isomiRs (such as the oligonucleotide represented by SEQ ID NOs: 58-68). The difference between a canonical miRNA and its isomiRs can be said lie only in their prevalence—generally, the most prevalent molecule is called the canonical miRNA, while the others are isomiRs. Dependent on the type, environment, position in its life cycle, or pathological state of a cell, individual isomiRs or miRNAs can be expressed at different levels; expression can even differ between population groups or gender (Loher et al., Oncotarget (2014) DOI: 10.18632/oncotarget.2405).

An antagomir of a miRNA molecule or mimic or isomiR or source thereof may be a nucleic acid, preferably a RNA which is complementary or reverse complementary to a part of the corresponding miRNA molecule or isomiR or mimic thereof. An antagomir preferably hybridizes with a part of the corresponding miRNA molecule or isomiR or mimic thereof. Preferred antagomir are complementary or reverse complementary to a part of sequences of mature miRNAs or isomiR of SEQ ID NOs: 51-125. A part may mean at least 50% of the length of the SEQ ID NO, at least 60%, at least 70%, at least 80%, at least 90% or 100%. In a preferred embodiment, an antagomir or a mimic thereof is complementary or reverse complementary to a seed sequence or a part of said seed sequence of a miRNA molecule or isomiR or mimic thereof. A part may mean at least 50% of the length of the seed sequence, at least 60%, at least 70%, at least 80%, at least 90% or 100%.

The chemical structure of the nucleotides of an antagomir of a miRNA molecule or mimics or sources thereof, or of a sense strand or an antisense strand in a mimic of a miRNA or of an isomiR, may be modified to increase stability, binding affinity and/or specificity. Said antagomir or sense strand or antisense strand may comprise or consists of a RNA molecule or preferably a modified RNA molecule. A preferred modified RNA molecule comprises a modified sugar. One example of such modification is the introduction of a 2′-O-methyl or 2′-O-methoxyethyl group or 2′ fluoride group on the nucleic acid to improve nuclease resistance and binding affinity to RNA. Another example of such modification is the introduction of a methylene bridge connecting the 2′-0 atom and the 4′-C atom of the nucleic acid to lock the conformation (Locked Nucleic Acid (LNA)) to improve affinity towards complementary single-stranded RNA. A third example is the introduction of a phosphorothioate group as linker between nucleic acid in the RNA-strand to improve stability against a nuclease attack. A fourth modification is conjugation of a lipophilic moiety on the 3′ end of the molecule, such as cholesterol to improve stability and cellular delivery. In a preferred embodiment, an antagomir of miRNA molecule consists of a fully LNA-modified phosphorotioate oligonucleotide. An antagomir as defined herein may comprise 1, 2, 3, 4, 5, 6, 7, 8, 9, 10 or more sugar modifications. It is also encompassed by the invention to introduce more than one distinct sugar modification in one antagomir.

In a preferred embodiment, the first two bases of a sense strand of a mimic have modified sugars, preferably 2′-O-methyl modifications. In a preferred embodiment, the first two of the last four bases of a sense strand of a mimic have modified sugars, preferably 2′-O-methyl modifications. In a preferred embodiment, the first two bases and the first two of the last four bases of a sense strand of a mimic have modified sugars, preferably 2′-O-methyl modifications. In a preferred embodiment, the last two bases of a sense strand of a mimic have modified sugars, preferably 2′-O-methyl modifications. In a preferred embodiment, the first two and the last two bases of a sense strand of a mimic have modified sugars, preferably 2′-O-methyl modifications. In a preferred embodiment, the last two bases of a sense strand of a mimic are DNA bases. In a preferred embodiment, the first two bases and the first two of the last four bases of a sense strand of a mimic have modified sugars, preferably 2′-O-methyl modifications, and the last two bases of said sense strand are DNA bases. In a preferred embodiment, the first two bases of a sense strand of a mimic have modified sugars, preferably 2′-O-methyl modifications, and the last two bases of said sense strand are DNA bases. In a preferred embodiment, the first two of the last four bases of a sense strand of a mimic have modified sugars, preferably 2′-O-methyl modifications, and the last two bases of said sense strand are DNA bases.

In preferred embodiments, this aspect provides the composition according to the invention,
wherein said miRNA shares at least 70% sequence identity with any one of SEQ ID NOs: 51-125, 209, 211, 213, 215, 217, 219, or 221,
and/or wherein said miRNA is from 15-30 nucleotides in length,
and/or wherein said source of a miRNA is a precursor of said miRNA and shares at least 70% sequence identity with any one of SEQ ID NOs: 1-16, preferably with any one of SEQ ID NOs: 1-8.

In preferred embodiments, this aspect provides the composition according to the invention, wherein said miRNA shares at least 70% sequence identity with any one of SEQ ID NOs: 51-125, 209, 211, 213, 215, 217, 219, or 221, and wherein said miRNA is from 15-30 nucleotides in length. In preferred embodiments, this aspect provides the composition according to the invention, wherein said miRNA shares at least 70% sequence identity with any one of SEQ ID NOs: 51-125, 209, 211, 213, 215, 217, 219, or 221, and wherein said miRNA is from 15-30 nucleotides in length and wherein said source of a miRNA is a precursor of said miRNA and shares at least 70% sequence identity with any one of SEQ ID NOs: 1-16, preferably with any one of SEQ ID NOs: 1-8. In preferred embodiments, this aspect provides the composition according to the invention, wherein said miRNA shares at least 70% sequence identity with any one of SEQ ID NOs: 51-125, 209, 211, 213, 215, 217, 219, or 221, and wherein said source of a miRNA is a precursor of said miRNA and shares at least 70% sequence identity with any one of SEQ ID NOs: 1-16, preferably with any one of SEQ ID NOs: 1-8.

A source of a miRNA molecule or a source of a mimic or an isomiR may be any molecule which is able to induce the production of a miRNA molecule or of a mimic or isomiR as identified herein and which preferably comprises a hairpin-like structure and/or a double stranded nucleic acid molecule. The presence of a hairpin-like structure may be assessed using the RNA shapes program (Steffen P. et al 2006) using sliding windows of 80, 100 and 120 nt or more. The hairpin-like structure is usually present in a natural or endogenous source of a miRNA molecule whereas a double-stranded nucleic acid molecule is usually present in a recombinant or synthetic source of a miRNA molecule or of an isomiR or mimic thereof.

A source of an antagomir of a miRNA molecule or a source of a mimic of an antagomir of a miRNA molecule may be any molecule which is able to induce the production of said antagomir, such as an appropriate vector.

A source of a miRNA molecule or of a mimic or an isomiR or an antagomir thereof may be a single stranded, a double stranded RNA or a partially double stranded RNA or may comprise three strands, an example of which is described in WO2008/10558. As used herein partially double stranded refers to double stranded structures that also comprise single stranded structures at the 5′ and/or at the 3′ end. It may occur when each strand of a miRNA molecule does not have the same length. In general, such partial double stranded miRNA molecule may have less than 75% double stranded structure and more than 25% single stranded structure, or less than 50% double stranded structure and more than 50% single stranded structure, or more preferably less than 25%, 20% or 15% double stranded structure and more than 75%, 80%, 85% single stranded structure.

Alternatively, a source of a miRNA molecule or of a mimic or an isomiR thereof is a DNA molecule encoding a precursor of a miRNA molecule or a mimic or an isomiR thereof. Preferred DNA molecules in this context are SEQ ID NOs: 9-16. The invention encompasses the use of a DNA molecule encoding a precursor of a miRNA molecule that has at least 70% identity with said SEQ ID NOs: 9-16. Preferably, the identity is at least 75%, 80%, 85%, 90%, 95%, 97%, 98%, 99% or 100%. Preferably in this embodiment, a DNA molecule has a length of at least 50, 55, 60, 70, 75, 80, 85, 90, 95, 100, 130, 150, 200, 250, 300, 350, 400 nucleotides or more and has at least 70% identity with a DNA sequence of SEQ ID NOs: 9-16.

The induction of the production of a given miRNA molecule or of a mimic or an isomiR, or the inductions of the production of a given antagomir thereof is preferably obtained when said source is introduced into a cell using one assay as defined below. Cells encompassed by the present invention are later on defined.

A preferred source of a miRNA molecule or of a mimic or an isomiR thereof is a precursor thereof, more preferably a nucleic acid encoding said miRNA molecule or a mimic or an isomiR thereof. A preferred precursor is a naturally-occurring precursor. A precursor may be a synthetic or recombinant precursor. A synthetic or recombinant precursor may be a vector that can express a naturally-occurring precursor. In preferred embodiments, this aspect provides the composition according to the invention, wherein a source of a miRNA is a precursor of a miRNA and is an oligonucleotide of at least 50 nucleotides in length.

A preferred precursor of a given miRNA molecule has a sequence represented by any one of SEQ ID NOs: 1-16. The invention encompasses the use of a precursor of a miRNA molecule or of an isomiR or mimic thereof that has at least 70% identity with said sequence. Preferably, identity is at least 75%, 80%, 85%, 90%, 95%, 97%, 98%, 99% or 100%. Preferably in this embodiment, a DNA molecule has a length of at least 50, 55, 60, 70, 75, 80, 85, 90, 95, 100, 130, 150, 200, 250, 300, 350, 400 nucleotides or more and has at least 70% identity with a sequence represented by any one of SEQ ID NOs: 1-16. Preferably, in this embodiment, a precursor comprises a seed sequence that shares at least 6 of the 7 nucleotides with a seed sequence selected from the group represented by SEQ ID NOs: 17-50. More preferably, a precursor comprises a seed sequence selected from the group represented by SEQ ID NOs: 17-50. A more preferred precursor of a given miRNA molecule has a sequence represented by any one of SEQ ID NOs: 1-8. The invention encompasses the use of a precursor of a miRNA molecule or of an isomiR or mimic thereof that has at least 70% identity with said sequence. Preferably, identity is at least 75%, 80%, 85%, 90%, 95%, 97%, 98%, 99% or 100%. Preferably in this embodiment, a DNA molecule has a length of at least 50, 55, 60, 70, 75, 80, 85, 90, 95, 100, 130, 150, 200, 250, 300, 350, 400 nucleotides or more and has at least 70% identity with a sequence represented by any one of SEQ ID NOs: 1-8. Preferably, in this embodiment, a precursor comprises a seed sequence that shares at least 6 of the 7 nucleotides with a seed sequence selected from the group represented by SEQ ID NOs: 17-50. More preferably, a precursor comprises a seed sequence selected from the group represented by SEQ ID NOs: 17-50.

Accordingly, a preferred source of a miRNA-323 molecule has at least 70%, 75%, 80%, 85%, 90%, 95%, 97%, 98%, 99% or 100% identity with SEQ ID NOs: 1 or 9, preferably SEQ ID NO: 1, and optionally has a length of at least 50, 55, 60, 70, 75, 80, 85, 90, 95, 100, 130, 150, 200, 250, 300, 350, 400 nucleotides or more and optionally comprises a seed sequence that shares at least 6 of the 7 nucleotides of any one of SEQ ID NOs: 17 or 24-28. Such a source is a precursor of a miRNA-323 molecule and of miRNA-323 isomiRs.

Accordingly, a preferred source of a miRNA-342 molecule has at least 70%, 75%, 80%, 85%, 90%, 95%, 97%, 98%, 99% or 100% identity with SEQ ID NOs: 2 or 10, preferably SEQ ID NO: 2, and optionally has a length of at least 50, 55, 60, 70, 75, 80, 85, 90, 95, 100, 130, 150, 200, 250, 300, 350, 400 nucleotides or more and optionally comprises a seed sequence that shares at least 6 of the 7 nucleotides of any one of SEQ ID NOs: 18 or 29-42. Such a source is a precursor of a miRNA-342 molecule and of miRNA-342 isomiRs.

Accordingly, a preferred source of a miRNA-520f molecule has at least 70%, 75%, 80%, 85%, 90%, 95%, 97%, 98%, 99% or 100% identity with SEQ ID NOs: 3 or 11, preferably SEQ ID NO: 3, and optionally has a length of at least 50, 55, 60, 70, 75, 80, 85, 90, 95, 100, 130, 150, 200, 250, 300, 350, 400 nucleotides or more and optionally comprises a seed sequence that shares at least 6 of the 7 nucleotides of any one of SEQ ID NOs: 19, 20, 43, or 44. Such a source is a precursor of a miRNA-520f molecule and of miRNA-520f isomiRs such as miRNA-520f-3p-i3.

Accordingly, a preferred source of a miRNA-3157 molecule has at least 70%, 75%, 80%, 85%, 90%, 95%, 97%, 98%, 99% or 100% identity with SEQ ID NOs: 4 or 12, preferably SEQ ID NO: 4, and optionally has a length of at least 50, 55, 60, 70, 75, 80, 85, 90, 95, 100, 130, 150, 200, 250, 300, 350, 400 nucleotides or more and optionally comprises a seed sequence that shares at least 6 of the 7 nucleotides of any one of SEQ ID NOs: 21 or 45-48. Such a source is a precursor of a miRNA-3157 molecule and of miRNA-3157 isomiRs.

Accordingly, a preferred source of a miRNA-193a molecule has at least 70%, 75%, 80%, 85%, 90%, 95%, 97%, 98%, 99% or 100% identity with SEQ ID NOs: 5 or 13, preferably SEQ ID NO: 5, and optionally has a length of at least 50, 55, 60, 70, 75, 80, 85, 90, 95, 100, 130, 150, 200, 250, 300, 350, 400 nucleotides or more and optionally comprises a seed sequence that shares at least 6 of the 7 nucleotides of any one of SEQ ID NOs: 22 or 49. Such a source is a precursor of a miRNA-193a molecule and of miRNA-193a isomiRs.

Accordingly, a preferred source of a miRNA-7 molecule has at least 70%, 75%, 80%, 85%, 90%, 95%, 97%, 98%, 99% or 100% identity with SEQ ID NOs: 6-8 or 14-16, preferably SEQ ID NOs: 6-8, and optionally has a length of at least 50, 55, 60, 70, 75, 80, 85, 90, 95, 100, 130, 150, 200, 250, 300, 350, 400 nucleotides or more and optionally comprises a seed sequence that shares at least 6 of the 7 nucleotides of any one of SEQ ID NOs: 23 or 50. Such a source is a precursor of a miRNA-7 molecule and of miRNA-7 isomiRs.

In this context, it is pointed that several precursors of a given mature miRNA molecule may lead to an identical miRNA molecule. For example, miRNA-7 may originate from precursor miRNA-7-1 or miRNA-7-2 or miRNA-7-3 (preferably identified as being SEQ ID NOs: 6, 8, or 8, respectively). Also in this context, it is pointed that several isomirs of a given mature miRNA molecule may lead to miRNA molecules with identical seed sequences. For example, mature miRNA-323-5p (SEQ ID NO: 51) and at least isomirs with SEQ ID NOs: 58 or 59 all share the same seed sequence (preferably identified as being SEQ ID NO: 17).

Preferred sources or precursors have been defined elsewhere herein. A preferred source includes or comprises an expression construct comprising a nucleic acid, i.e. DNA encoding said precursor of said miRNA or encoding said antagomir, more preferably said expression construct is a viral gene therapy vector selected from gene therapy vectors based on an adenovirus, an adeno-associated virus (AAV), a herpes virus, a pox virus and a retrovirus. A preferred viral gene therapy vector is an AAV or Lentiviral vector. Other preferred vectors are oncolytic viral vectors. Such vectors are further described herein below. Alternatively, a source may be a synthetic miRNA molecule or a chemical mimic as further defined in the part dedicated to general definitions.

In preferred embodiments, this aspect provides the nanoparticle composition according to the invention, further comprising a further miRNA or antagomir selected from the group consisting of miRNA-193a, miRNA-323, miRNA-342, miRNA-520f, miRNA-520f-i3, miRNA-3157, and miRNA-7, or an isomiR thereof, or a mimic thereof, or an antagomir thereof. Accordingly, in preferred embodiments this aspect provides a composition further comprising:

i) one or more of a miRNA-323, miRNA-342, miRNA-520f, miRNA-520f-i3, miRNA-3157, and miRNA-7, or an isomiR thereof, or a mimic thereof, or an antagomir thereof, or

ii) one or more of a miRNA-193a, miRNA-342, miRNA-520f, miRNA-520f-i3, miRNA-3157, and miRNA-7, or an isomiR thereof, or a mimic thereof, or an antagomir thereof, or

iii) one or more of a miRNA-193a, miRNA-323, miRNA-520f, miRNA-520f-i3, miRNA-3157, and miRNA-7 or an isomiR thereof, or a mimic thereof, or an antagomir thereof, or

iv) one or more of a miRNA-193a, miRNA-323, miRNA-342, miRNA-520f-i3, miRNA-3157, and miRNA-7, or an isomiR thereof, or a mimic thereof, or an antagomir thereof, or

v) one or more of a miRNA-193a, miRNA-323, miRNA-342, miRNA-520f, miRNA-3157, and miRNA-7, or an isomiR thereof, or a mimic thereof, or an antagomir thereof, or

vi) one or more of a miRNA-193a, miRNA-323, miRNA-342, miRNA-520f, miRNA-520f-i3, and miRNA-7, or an isomiR thereof, or a mimic thereof, or an antagomir thereof, or

vii) one or more of a miRNA-193a, miRNA-323, miRNA-342, miRNA-520f, miRNA-520f-i3, and miRNA-3157, or an isomiR thereof, or a mimic thereof, or an antagomir thereof, or

viii) one or more of a miRNA-193a, miRNA-323, miRNA-342, miRNA-3157, and miRNA-7, or an isomiR thereof, or a mimic thereof, or an antagomir thereof.

Accordingly, in more preferred embodiments this aspect provides a composition further comprising:

i) one or more of a miRNA-323, miRNA-342, miRNA-520f, miRNA-520f-i3, miRNA-3157, and miRNA-7, or an isomiR thereof, or a mimic thereof, or

ii) one or more of a miRNA-193a, miRNA-342, miRNA-520f, miRNA-520f-i3, miRNA-3157, and miRNA-7, or an isomiR thereof, or a mimic thereof, or

iii) one or more of a miRNA-193a, miRNA-323, miRNA-520f, miRNA-520f-i3, miRNA-3157, and miRNA-7 or an isomiR thereof, or a mimic thereof, or

iv) one or more of a miRNA-193a, miRNA-323, miRNA-342, miRNA-520f-i3, miRNA-3157, and miRNA-7, or an isomiR thereof, or a mimic thereof, or

v) one or more of a miRNA-193a, miRNA-323, miRNA-342, miRNA-520f, miRNA-3157, and miRNA-7, or an isomiR thereof, or a mimic thereof, or

vi) one or more of a miRNA-193a, miRNA-323, miRNA-342, miRNA-520f, miRNA-520f-i3, and miRNA-7, or an isomiR thereof, or a mimic thereof, or

vii) one or more of a miRNA-193a, miRNA-323, miRNA-342, miRNA-520f, miRNA-520f-i3, and miRNA-3157, or an isomiR thereof, or a mimic thereof, or

viii) one or more of a miRNA-193a, miRNA-323, miRNA-342, miRNA-3157, and miRNA-7, or an isomiR thereof, or a mimic thereof, or an antagomir thereof.

Nanoparticle Composition

The composition can further comprise solvents and/or excipients, preferably pharmaceutically acceptable excipients. Preferred solvents are aqueous solutions such as pharmaceutically acceptable buffers, for example PBS or citrate buffer. A preferred citrate buffer comprises 50 mM citrate at pH 2.5-3.5 such as pH 3, preferably set using NaOH. A preferred PBS is at pH 7-8 such as pH 7.4. PBS preferably does not comprise bivalent cations such as Ca²⁺ and Mg²⁺. Another preferred pharmaceutically acceptable excipient is ethanol. Most preferably, the composition comprises a physiological buffer such as PBS or a Good's buffer or Hepes-buffered saline or Hank's balanced salt solution or Ringer's balanced salt solution or a Tris buffer. Preferred compositions are pharmaceutical compositions.

The composition can comprise further excipients. These further excipients can be comprised in the nanoparticles.

In preferred embodiments, this aspect provides the composition according to the invention, further comprising a sterol, preferably selected from the group consisting of adosterol, brassicasterol, campesterol, cholecalciferol, cholestenedione, cholestenol, cholesterol, delta-7-stigmasterol, delta-7-avenasterol, dihydrotachysterol, dimethylcolesterol, ergocalciferol, ergosterol, ergostenol, ergostatrienol, ergostadienol, ethylcholestenol, fusidic acid, lanosterol, norcholestadienol, β-sitosterol, spinasterol, stigmastanol, stigmastenol, stigmastadienol, stigmastadienone, stigmasterol, and stigmastenone, more preferably cholesterol. More particularly, in preferred embodiments, this aspect provides the composition according to the invention, wherein the nanoparticles further comprise a sterol, preferably selected from the group consisting of adosterol, brassicasterol, campesterol, cholecalciferol, cholestenedione, cholestenol, cholesterol, delta-7-stigmasterol, delta-7-avenasterol, dihydrotachysterol, dimethylcolesterol, ergocalciferol, ergosterol, ergostenol, ergostatrienol, ergostadienol, ethylcholestenol, fusidic acid, lanosterol, norcholestadienol, β-sitosterol, spinasterol, stigmastanol, stigmastenol, stigmastadienol, stigmastadienone, stigmasterol, and stigmastenone, more preferably cholesterol.

Preferably, such a further comprised sterol is not conjugated to any moiety. Conjugated sterols can also be comprised, as will be explained later herein. As such, both conjugated and unconjugated sterols can be comprised. Unless explicitly indicated otherwise, reference to a sterol is intended as reference to an unconjugated sterol.

When a sterol is comprised in the composition, it is preferably comprised in the nanoparticle, and preferably at least 5, 10, 15, 20, 25, 30, 35, 40, 45, 50, 55, 60, 65, or 70 mol % of sterol is comprised; preferably at most 80, 75, 70, 65, 60, 65, 50, 45, 40, 35, or 30 mol % of sterol is comprised. As explained above, this molar percentage only pertains to the substances making up the lipid nanoparticle, and not to solvents or cargo such as oligonucleotides. When a sterol is comprised in the composition, preferably 5 to 70 mol %, 15 to 60 mol %, 25 to 60 mol %, 35 to 60 mol %, 40 to 60 mol %, or 45 to 55 mol % is comprised; more preferably 40 to 60 mol % or 45 to 55 mol % is comprised, most preferably 45 to 55 mol % is comprised, such as 48 mol % or 54 mol %.

In preferred embodiments, this aspect provides the composition according to the invention, further comprising a phospholipid, preferably selected from the group consisting of distearoyl phosphatidylcholine (DSPC), dipalmitoyl phosphatidylcholine (DPPC), dimyristoyl phosphatidylcholine (DMPC), dilauroyl phosphatidylcholine (DLPC), dioleyl phosphatidylcholine (DOPC), 1,2-dioleoyl-sn-glycero-3-phosphoethanolamine (DOPE), egg phosphatidylcholine (EggPC), soy phosphatidylcholine (SoyPC), more preferably distearoyl phosphatidylcholine (DSPC). More particularly, in preferred embodiments, this aspect provides the composition according to the invention, wherein the nanoparticles further comprise a phospholipid, preferably selected from the group consisting of distearoyl phosphatidylcholine (DSPC), dipalmitoyl phosphatidylcholine (DPPC), dimyristoyl phosphatidylcholine (DMPC), dilauroyl phosphatidylcholine (DLPC), dioleyl phosphatidylcholine (DOPC), 1,2-dioleoyl-sn-glycero-3-phosphoethanolamine (DOPE), egg phosphatidylcholine (EggPC), soy phosphatidylcholine (SoyPC), more preferably distearoyl phosphatidylcholine (DSPC).

Preferably, such a further comprised phospholipid is not conjugated to any moiety. Conjugated phospholipids can also be comprised, as will be explained later herein. As such, both conjugated and unconjugated phospholipids can be comprised.

When a phospholipid is comprised in the composition, it is preferably comprised in the nanoparticle, and preferably at least 1, 2, 3, 4, 5, 6, 7, 8, 9, 10, 11, 12, 13, 14, 15, 16, 17, 18, 19, 20, 25, 30, 35, 40, 45, 50, 55, or 60 mol % of phospholipid is comprised; preferably at most 65, 60, 55, 50, 45, 40, 35, 30, 25, 20, 15, 14, 13, 12, 11, 10, 9, 8, 7, 6, or 5 mol % of phospholipid is comprised. As explained above, this molar percentage only pertains to the substances making up the lipid nanoparticle, and not to solvents or cargo such as oligonucleotides. When a phospholipid is comprised in the composition, preferably 0 to 40 mol %, 0 to 35 mol %, 0 to 30 mol %, 5 to 30 mol %, 5 to 25 mol %, or 5 to 20 mol % is comprised; more preferably 5 to 20 mol % or 5 to 15 mol % is comprised, most preferably 5 to 15 mol % is comprised, such as 10 mol % or 11 mol %.

In preferred embodiments, this aspect provides the composition according to the invention, further comprising a conjugate of a water soluble polymer and a lipophilic anchor, wherein:
i) the water soluble polymer is selected from the group consisting of poly(ethylene glycol) (PEG), poly(hydroxyethyl-l-asparagine) (PHEA), poly-(hydroxyethyl-L-glutamine) (PHEG), poly(glutamic acid) (PGA), polyglycerol (PG), poly(acrylamide) (PAAm), poly(vinylpyrrolidone) (PVP), poly(N-(2-hydroxypropyl)methacrylamide) (PHPMA), and poly(2-oxazoline) (POx) such as poly(2-methyl-2-oxazoline) (PMeOx) and poly(2-ethyl-2-oxazoline) (PEtOx), or copolymers thereof
   and wherein
ii) the lipophilic anchor is selected from the group consisting of a sterol, a lipid, and a vitamin E derivative. Preferably, the lipophilic anchor is a lipid, more preferably a diglyceride.

More particularly, in preferred embodiments, this aspect provides the composition according to the invention, wherein the nanoparticles further comprise a conjugate of a water soluble polymer and a lipophilic anchor as described above. The water soluble polymer generally increases the colloidal stability of the nanoparticles, to which is it linked via the lipophilic anchor. In general, the lipophilic anchor embeds in the lipid bilayer or in the micelle, and thus links the water soluble polymer to the surface of the nanoparticle. The use of such water soluble polymers for this purpose is known in the art (Knop et al., 2010, doi: 10.1002/anie.200902672). A preferred water soluble polymer is poly(ethylene glycol). Preferably, the water soluble polymer has a molecular weight ranging from about 750 Da to about 15000 Da, more preferably from about 1000 Da to about 6000 Da, even more preferably from about 1000 Da to about 3000 Da, most preferably from about 1500 Da to about 3000 Da, such as about 2000 Da. Accordingly, PEG-2000 is a preferred water soluble polymer for use in a conjugate as described above. The water soluble polymer is preferably a linear polymer, and is preferably conjugated at one of its two termini. The other terminus is preferably uncharged at physiological conditions, such as a hydroxyl group or a methyl or ethyl ether. Preferably, the non-conjugated terminus is a methyl ether or a hydroxyl group, most preferably a methyl ether.

The lipophilic anchor to which the water soluble polymer is conjugated generally serves to ensure a connection between the water soluble polymer and the nanoparticle. The method of conjugation between the polymer and the anchor is not important, a skilled person can select any suitable chemical bond such as an ester bond, an amide bond, an ether linkage, a triazole, or any other moiety resulting from conjugating a water soluble polymer to a lipophilic anchor. The use of small linkers is also envisaged, such as succinic acid or glutaric acid. The lipophilic anchor is selected from the group consisting of a sterol, a lipid, and a vitamin E derivative. Preferred sterols are described above. Preferred vitamin E derivatives are tocopherols and tocotrienols such as alpha-tocopherol, beta-tocopherol, gamma-tocopherol, delta-tocopherol, and corresponding tocotrienols. Preferably, the lipophilic anchor is a lipid, more preferably a diglyceride or a phospholipid. Examples of preferred lipids are described above, examples of preferred diglycerides are distearoylglycerol, preferably 1,2-distearoyl-sn-glycerol, dipalmitoylglycerol, preferably 1,2-dipalmitoyl-sn-glycerol, dioleoylglycerol, preferably 1,2-dioleoyl-sn-glycerol, and diarachidoylglycerol, preferably 1,2-diarachidoyl-sn-glycerol. A most preferred diglyceride is distearoylglycerol, preferably 1,2-distearoyl-sn-glycerol.

Suitable examples of conjugates as described above are (1,2-distearoyl-sn-glycerol)-[methoxy(polyethylene glycol-2000)] ether, (1,2-distearoyl-sn-glycerol)-[methoxy(polyethylene glycol-1500)] ether, (1,2-distearoyl-sn-glycerol)-[methoxy(polyethylene glycol-3000)]ether, (1,2-distearoyl-sn-glycerol)-[hydroxy(polyethylene glycol-2000)]ether, (1,2-distearoyl-sn-glycerol)-[hydroxy(polyethylene glycol-1500)]ether, (1,2-distearoyl-sn-glycerol)-[hydroxy(polyethylene glycol-3000)]ether, (1,2-distearoyl-sn-glyceryl)-[methoxy(polyethylene glycol-2000)carboxylate], (1,2-distearoyl-sn-glyceryl)-[methoxy(polyethylene glycol-1500) carboxylate], (1,2-distearoyl-sn-glyceryl)-[methoxy(polyethylene glycol-3000) carboxylate], (1,2-distearoyl-sn-glyceryl)-[hydroxy(polyethylene glycol-2000) carboxylate], (1,2-distearoyl-sn-glyceryl)-[hydroxy(polyethylene glycol-1500) carboxylate], (1,2-distearoyl-sn-glyceryl)-[hydroxy(polyethylene glycol-3000) carboxylate], (1,2-distearoyl-sn-glyceryl)-[methoxy(polyethylene glycol-2000) carbamate], (1,2-distearoyl-sn-glyceryl)-[methoxy(polyethylene glycol-1500) carbamate], (1,2-distearoyl-sn-glyceryl)-[methoxy(polyethylene glycol-3000) carbamate], (1,2-distearoyl-sn-glyceryl)-[hydroxy(polyethylene glycol-2000) carbamate], (1,2-distearoyl-sn-glyceryl)-[hydroxy(polyethylene glycol-1500) carbamate], and (1,2-distearoyl-sn-glyceryl)-[hydroxy(polyethylene glycol-3000) carbamate], wherein the stearoyl moieties can optionally be replaced by other fatty acids, preferably by other 010-020 fatty acids. For carbamates and esters as described above, the parent amines and parent alcohols and parent carboxylic acids can also be switched around, for example a PEG-alcohol can be reacted with a carboxylic acid analogue of a diglyceride. Most preferred examples of conjugates are (1,2-distearoyl-sn-glycerol)-[methoxy(polyethylene glycol-2000)] ether, which is also known as DSG-PEG (CAS #: 308805-39-2), and its ester analogue (1,2-distearoyl-sn-glyceryl)-[methoxy(polyethylene glycol-2000)carboxylate], and its carbamate analogue (1,2-distearoyl-sn-glyceryl)-[methoxy(polyethylene glycol-2000) carbamate] or 1,2-distearoyloxy propylamine 3-N-methoxy(polyethylene glycol)-2000 carbamoyl which is also known as DSA-PEG, and its amide analogue.

When a conjugate as described above is comprised in the composition, it is preferably comprised in the nanoparticle, and preferably at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 4.5, or 5.0 mol % of conjugate is comprised; preferably at most 6.5, 6.0, 5.5, 5.0, 4.5, 4.0, 3.5, 3.0, 2.5, 2.0, 1.9, 1.8, 1.7, 1.6, 1.5, 1.4, 1.3, 1.2, 1.1, 1.0, 0.9, 0.8, 0.7, 0.6, or 0.5 mol % of conjugate is comprised. As explained above, this molar percentage t only pertains to the substances making up the lipid nanoparticle, and not to solvents or cargo such as oligonucleotides. When a conjugate is comprised in the composition, preferably 0 to 4 mol %, 0 to 3 mol %, 0.3 to 3 mol %, 0.5 to 3 mol %, 0.5 to 2.5 mol %, or 1 to 2.5 mol % is comprised; more preferably 0.5 to 2.5 mol % or 0.7 to 2.5 mol % is comprised, most preferably 0.8 to 2.4 mol % is comprised, such as 1 mol % or 2 mol %.

Preferred nanoparticles comprise a diamino lipid and a sterol. Further preferred nanoparticles comprise a diamino lipid and a phospholipid. Further preferred nanoparticles comprise a diamino lipid and a conjugate of a water soluble polymer and a lipophilic anchor. Preferred nanoparticles comprise a diamino lipid and a sterol and a phospholipid. Preferred nanoparticles comprise a diamino lipid and a sterol and a conjugate of a water soluble polymer and a lipophilic anchor. Preferred nanoparticles comprise a diamino lipid and a phospholipid and a conjugate of a water soluble polymer and a lipophilic anchor. Most preferred nanoparticles comprise a diamino lipid and a sterol and a phospholipid and a conjugate of a water soluble polymer and a lipophilic anchor.

In preferred embodiments, this aspect provides the composition according to the invention, wherein the nanoparticles comprise:
i) 20-60 mol % of diamino lipid, and
ii) 0-40 mol % of phospholipid, and
iii) 30-70 mol % of a sterol, preferably cholesterol, and
iv) 0-10 mol % of a conjugate of a water soluble polymer and a lipophilic anchor as defined above.
   In further preferred embodiments the nanoparticles comprise
i) 25-55 mol % of diamino lipid, and
ii) 1-30 mol % of phospholipid, and
iii) 35-65 mol % of a sterol, preferably cholesterol, and
iv) 0.1-4 mol % of a conjugate of a water soluble polymer and a lipophilic anchor.
   In further preferred embodiments the nanoparticles comprise
i) 30-50 mol % of diamino lipid, and
ii) 5-15 mol % of phospholipid, and
iii) 40-60 mol % of a sterol, preferably cholesterol, and
iv) 0.5-2.5 mol % of a conjugate of a water soluble polymer and a lipophilic anchor.
   In further preferred embodiments the nanoparticles comprise
i) about 38-42 mol % of diamino lipid, and
ii) about 8-12 mol % of phospholipid, and
iii) about 46-50 mol % of a sterol, preferably cholesterol, and
iv) about 1.8-2.2 mol % of a conjugate of a water soluble polymer and a lipophilic anchor.

### Medical Use

The invention provides the medical use of these nanoparticles, and of miRNA from the composition. Accordingly, this aspect provides the use of a composition according to the invention, for use as a medicament. Accordingly, this aspect provides the use of a miRNA from the composition, for use as a medicament. This use can also be the use of the composition or miRNA in the manufacture of a medicament. In preferred embodiments, the composition according to the invention or the miRNA from the composition is for use as a medicament, preferably in the treatment of cancer. This may be for use as a medicament for preventing, treating, reverting, curing and/or delaying a cancer, or in other words for obtaining an anti-tumor effect. Such a composition for use is referred to hereinafter as a composition for use according to the invention. For medical use as described herein, a preferred miRNA from the composition is miRNA-193a or a mimic or isomiR or precursor thereof, and preferred compositions according to the invention comprise miRNA-193a or a mimic or isomiR or precursor thereof.

Preferred cancers in this context are colorectal cancer, colon cancer, head and neck cancer, glioblastoma, brain tumour, cervix cancer, carcinoma, tumours of the haematopoietic and lymphoid malignancies, liver cancer, breast cancer such as triple negative breast cancer, prostate cancer, bladder cancer, ovarian cancer, lung cancer, renal cell cancer, pancreas cancer, or melanoma, more preferred are colorectal cancer, colon cancer, head and neck cancer, glioblastoma, brain tumour, cervix cancer, carcinoma, tumours of the haematopoietic and lymphoid malignancies, liver cancer, breast cancer such as triple negative breast cancer, or melanoma, still more preferably carcinoma, tumours of the haematopoietic and lymphoid malignancies, liver cancer, breast cancer such as triple negative breast cancer, or melanoma, even more preferably liver cancer such as hepatocellular carcinoma (HCC), lung cancer such as non-small-cell lung carcinoma (NSCLC), tumours of the haematopoietic and lymphoid malignancies such as leukemia or lymphoma or myeloma wherein leukemia is preferred, breast cancer such as triple-negative breast cancer (TNBC), melanoma, pancreas cancer, colon cancer, renal cell cancer (RCC), squamous cell carcinoma such as head and neck cancer (HNSCC), prostate cancer, and carcinoma such as hepatocellular carcinoma (HCC) or non-small-cell lung carcinoma or squamous cell carcinoma. Examples of leukemias are acute lymphoblastic leukemia (ALL), acute myelogenous leukemia (AML), chronic lymphocytic leukemia (CLL), small lymphocytic lymphoma (SLL), chronic myelogenous leukemia (CML), myelodysplastic syndrome, and acute monocytic leukemia (AMoL), wherein AML is preferred. Examples of lymphomas are cutaneous T cell lymphoma (CTCL), B cell lymphoma, hodgkin's lymphomas (all four subtypes, i.e. nodular sclerosing, mixed-cellularity, lymphocyte-rich, and lymphocyte-depleted), and non-Hodgkin's lymphomas (and its subtypes). Myeloma is also known as multiple myeloma, also known as plasma cell myeloma.

In a preferred embodiment, an anti-tumour activity is assessed in tumour cells of a subject. More preferably, said tumour cells are HNSCC cells (Head and Neck Squamous Cell Carcinoma), i.e. squamous cell carcinomas or mucosal or epithelium cells of the upper aerodigestive tract including the lip, inner lip, oral cavity (mouth), tongue, floor of mouth, gingiva, hard palate, nasal cavity (inside the nose), paranasal sinuses, pharynx, including the nasopharynx, oropharynx, hypopharynx and larynx (i.e. laryngeal cancer including glottic, supraglottic and subglottic cancer), trachea. Alternatively, said tumour cells may be colorectal cells, colon cells, brain cells, glioblastoma cells, breast cells, cervical cells.

In a preferred embodiment the cancer is colorectal cancer. In a preferred embodiment the cancer is colon cancer. In a preferred embodiment the cancer is head and neck cancer. In a preferred embodiment the cancer is glioblastoma. In a preferred embodiment the cancer is brain tumour. In a preferred embodiment the cancer is breast cancer such as triple negative breast cancer. In a preferred embodiment the cancer is cervix cancer. In a preferred embodiment the cancer is carcinoma. In a preferred embodiment the cancer is a tumour of the haematopoietic or a lymphoid malignancy. In a preferred embodiment the cancer is liver cancer. In a preferred embodiment the cancer is prostate cancer. In a preferred embodiment the cancer is bladder cancer. In a preferred embodiment the cancer is ovarian cancer. In a preferred embodiment the cancer is lung cancer. In a preferred embodiment the cancer is renal cell cancer. In a preferred embodiment the cancer is pancreas cancer. In a preferred embodiment the cancer is melanoma.

Unless otherwise indicated, an anti-tumor effect is preferably assessed or detected before treatment and after at least one week, two weeks, three weeks, four weeks, one month, two months, three months, four months, five months, six months or more in a treated subject. An anti-tumor effect is preferably identified in a subject as:
an inhibition of proliferation or a detectable decrease of proliferation of tumor cells or a decrease in cell viability of tumor cells or melanocytes, and/or
an increase in the capacity of differentiation of tumor cells, and/or
an increase in tumor cell death, which is equivalent to a decrease in tumor cell survival, and/or
a delay in occurrence of metastases and/or of tumor cell migration, and/or
an inhibition or prevention or delay of the increase of a tumor weight or growth, and/or
a prolongation of patient survival of at least one month, several months or more (compared to those not treated or treated with a control or compared with the subject at the onset of the treatment), and/or
a decrease in tumor size or volume.

In the context of the invention, a patient may survive and may be considered as being disease free. Alternatively, the disease or condition may have been stopped or delayed or regressed. An inhibition of the proliferation of tumor cells may be at least 20%, 30%, 40%, 50%, 55%, 60%, 65%, 70% or 75% or more. Proliferation of cells may be assessed using known techniques. An decrease in cell viability of tumor cells or melanocytes may be a decrease of at least 20%, 30%, 40%, 50%, 55%, 60%, 65%, 70% or 75% or more. Such decrease may be assessed 4 days after transfection with a given miRNA molecule, equivalent or source thereof. Cell viability may be assessed via known techniques such as the MTS assay.

Treatment of cancer can be the reduction of tumour volume or a decrease of tumour cell viability. Reduction of tumour volume can be assessed using a caliper. A decrease of tumour volume or cell viability or survival may be at least a decrease of at least 1%, 5%, 10%, 15%, 20%, 25%, 30%, 40%, 50%, 55%, 60%, 65%, 70% or 75%, or more. An induction of apoptosis in tumour cells or an induction of tumour cell death may be at least 1%, 5%, 10%, 15%, 20%, 25%, 30%, 40%, 50%, 55%, 60%, 65%, 70% or 75%, or more. Tumour cell viability or survival or death may be assessed using techniques known to the skilled person. Tumour cell viability and death may be assessed using routine imaging methods such MRI, CT or PET, and derivatives thereof, or in biopsies. Tumour cell viability may be assessed by visualising the extension of the lesion at several time points. A decrease of 10%,15%, 20%, 25%, 30%, 40%, 50%, 55%, 60%, 65%, 70% or 75%, or more of the lesion observed at least once will be seen as a decrease of tumour cell viability.

An inhibition of the proliferation of tumour cells may be at least 1%, 5%, 10%, 15%, 20%, 30%, 40%, 50%, 55%, 60%, 65%, 70% or 75%, or more. Proliferation of cells may be assessed using known techniques as a standard proliferation assay. Such a proliferation assay may use of vital stains such as Cell Titer Blue (Promega). This includes a substrate molecule that is converted into a fluorescent molecule by metabolic enzymes. The level of fluorescence then reflects the number of living and metabolically active cells. Alternatively, such proliferation assay may determine the mitotic index. The mitotic index is based on the number of tumor cells under proliferation stage compared to the number of total tumor cells. The labelling of proliferative cells can be performed by using the antibody Ki-67 and immunohistochemistry staining. An inhibition of the proliferation of tumours cells may be seen when the mitotic index is reduced by at least 20%, at least 30%, at least 50% or more (as described in Kearsley J. H., et al, 1990, PMID: 2372483).

A delay in occurrence of metastases and/or of tumor cell migration may be a delay of at least one week, one month, several months, one year or longer. The presence of metastases may be assessed using MRI, CT or Echography or techniques allowing the detection of circulating tumour cells (CTC). Examples of the latter tests are CellSearch CTC test (Veridex), an EpCam-based magnetic sorting of CTCs from peripheral blood.

In certain embodiments, an inhibition or a decrease of a tumour weight or a delayed tumour growth or an inhibition of a tumour growth may be of at least 1%, 5%, 10%, 15%, 20%, 30%, 40%, 50%, 55%, 60%, 65%, 70% or 75%, or more. Tumour weight or volume tumour growth may be assessed using techniques known to the skilled person. The detection of tumour growth or the detection of the proliferation of tumour cells may be assessed in vivo by measuring changes in glucose utilization by positron emission tomography with the glucose analogue 2-[18F]-fluor-2-deoxy-D-glucose (FDG-PET) or [18F]-'3-fluoro-'3-deoxy-L-thymidine PET. An ex vivo alternative may be staining of a tumour biopsy with Ki67.

An increase in the capacity of differentiation of tumor cells may be assessed using a specific differentiation marker and following the presence of such marker on cells treated. Preferred markers or parameters are p16, Trp-1 and PLZF, c-Kit, MITF, Tyrosinase, and Melanin. This may be done using RT-PCR, western blotting or immunohistochemistry. An increase of the capacity of differentiation may be at least a detectable increase after at least one week of treatment using any of the identified techniques. Preferably, the increase is of 1%; 5%, 10%, 15%, 20%, 25%, or more, which means that the number of differentiated cells within a given sample will increase accordingly. In certain embodiments, tumor growth may be delayed at least one week, one month, two months or more. In a certain embodiment, an occurrence of metastases is delayed at least one week, two weeks, three weeks, four weeks, one months, two months, three months, four months, five months, six months or more.

Reductions to practice of exemplary embodiments of these methods or medical uses are shown in the examples.

The invention provides an in vivo, in vitro, or ex vivo method for stimulating cellular uptake of a miRNA, the method comprising the step of contacting a cell with a composition according to the invention.

The method can further encompass allowing the nanoparticles of the invention to actively or passively enter a cell, preferably by passing over the cell membrane. The method is preferably for increasing the efficiency of a miRNA for use in treatment. Reductions to practice of exemplary embodiments of these methods or medical uses are shown in the examples.

In preferred embodiments, the composition according to the invention or the miRNA from the composition is for use in the treatment of cancer. Accordingly, the invention provides the miRNA from the composition for the treatment of cancer, such as a miRNA molecule, an isomiR, a mimic, or a precursor of a miRNA molecule, an isomiR, or a mimic as described earlier herein.

In preferred embodiments, the composition according to the invention or the miRNA from the composition is for use in the treatment of chemotherapy-resistant cancer such as sorafenib-resistant cancer.

In preferred embodiments, the composition according to the invention or the miRNA from the composition is for use in the treatment of carcinoma. More preferably, the composition according to the invention or the miRNA from the composition is for use in the treatment of chemotherapy-resistant carcinoma such as sorafenib-resistant carcinoma.

In preferred embodiments, the composition according to the invention or the miRNA from the composition is for use in the treatment of hepatocellular carcinoma (HCC). More preferably, the composition according to the invention or the miRNA from the composition is for use in the treatment of chemotherapy-resistant HCC such as hepatocellular carcinoma (HCC) that is resistant to receptor tyrosine kinase inhibitors such as VEGF receptor inhibitors, for example axitinib, cediranib, lenvatinib, nintedanib, pazopanib, regorafenib, semaxanib, sorafenib, sunitinib, tivozanib, toceranib, or vandetanib, preferably sorafenib.

In preferred embodiments, the composition according to the invention or the miRNA from the composition is for use in the treatment of non-small-cell lung carcinoma (NSCLC). More preferably, the composition according to the invention or the miRNA from the composition is for use in the treatment of chemotherapy-resistant NSCLC such as NSCLC that is resistant to platinum-based cell-cycle nonspecific antineoplastic agents (for example carboplatin, cisplatin, dicycloplatin, nedaplatin, oxaliplatin, or satraplatin, preferably cisplatin or carboplatin), or that is resistant to taxanes (for example cabazitaxel, docetaxel, larotaxel, ortataxel, paclitaxel, or tesetaxel, preferably paclitaxel or docetaxel, more preferably paclitaxel), or that is resistant to pyrimidine-based antimetabolites (for example fluorouracil, capecitabine, doxifluridine, tegafur, carmofur, floxuridine, cytarabine, gemcitabine, azacitidine, or decitabine, preferably gemcitabine), or that is resistant to vinca alkaloids (for example vinblastine, vincristine, vinflunine, vindesine, or vinorelbine, preferably vinorelbine), or that is resistant to folic acid antimetabolites (aminopterin, methotrexate, pemetrexed, pralatrexate, or raltitrexed, preferably pemetrexed).

In preferred embodiments, the composition according to the invention or the miRNA from the composition is for use in the treatment of triple-negative breast cancer (TNBC). More preferably, the composition according to the invention or the miRNA from the composition is for use in the treatment of chemotherapy-resistant TNBC such as anthracyclin-resistant TNBC, for example TNBC resistant to aclarubicin, daunorubicin, doxorubicin, epirubicin, idarubicin, amrubicin, pirarubicin, valrubicin, or zorubicin, preferably to doxorubicin.

In preferred embodiments, the composition according to the invention or the miRNA from the composition is for use in the treatment of melanoma. More preferably, the composition according to the invention or the miRNA from the composition is for use in the treatment of chemotherapy-resistant melanoma such as melanoma that is resistant to nonclassical cell-cycle nonspecific antineoplastic agents (for example procarbazine, dacarbazine, temozolomide, altretamine, mitobronitol, or pipobroman, preferably dacarbazine or temozolomide), or that is resistant to taxanes (for example cabazitaxel, docetaxel, larotaxel, ortataxel, paclitaxel, or tesetaxel, preferably paclitaxel such as albumin-bound paclitaxel), or that is resistant to platinum-based cell-cycle nonspecific antineoplastic agents (for example carboplatin, cisplatin, dicycloplatin, nedaplatin, oxaliplatin, or satraplatin, preferably cisplatin or carboplatin), or that is resistant to vinca alkaloids (for example vinblastine, vincristine, vinflunine, vindesine, or vinorel bine, preferably vinblastine).

In preferred embodiments, the composition according to the invention or the miRNA from the composition is for use in the treatment of pancreas cancer. More preferably, the composition according to the invention or the miRNA from the composition is for use in the treatment of chemotherapy-resistant pancreas cancer such as pancreas cancer that is resistant to taxanes (for example cabazitaxel, docetaxel, larotaxel, ortataxel, paclitaxel, or tesetaxel, preferably paclitaxel such as albumin-bound paclitaxel), or that is resistant to pyrimidine-based antimetabolites (for example fluorouracil, capecitabine, doxifluridine, tegafur, carmofur, floxuridine, cytarabine, gemcitabine, azacitidine, or decitabine, preferably fluorouracil or gemcitabine), or that is resistant to topoisomerase inhibitors (for example camptothecin, cositecan, belotecan, gimatecan, exatecan irinotecan, lurtotecan, silatecan, topotecan, rubitecan, preferably irinotecan).

In preferred embodiments, the composition according to the invention or the miRNA from the composition is for use in the treatment of colon cancer. More preferably, the composition according to the invention or the miRNA from the composition is for use in the treatment of chemotherapy-resistant colon cancer such as colon cancer that is resistant to pyrimidine-based antimetabolites (for example fluorouracil, capecitabine, doxifluridine, tegafur, carmofur, floxuridine, cytarabine, gemcitabine, azacitidine, or decitabine, preferably fluorouracil or capecitabine), or that is resistant to topoisomerase inhibitors (for example camptothecin, cositecan, belotecan, gimatecan, exatecan irinotecan, lurtotecan, silatecan, topotecan, rubitecan, preferably irinotecan), or that is resistant to platinum-based cell-cycle nonspecific antineoplastic agents (for example carboplatin, cisplatin, dicycloplatin, nedaplatin, oxaliplatin, or satraplatin, preferably oxaliplatin), or that is resistant to trifluridine or tipiracil, or a combination of trifluridine and tipiracil.

In preferred embodiments, the composition according to the invention or the miRNA from the composition is for use in the treatment of renal cell cancer (RCC). More preferably, the composition according to the invention or the miRNA from the composition is for use in the treatment of chemotherapy-resistant RCC such as RCC that is resistant to receptor tyrosine kinase inhibitors such as VEGF receptor inhibitors, for example axitinib, cediranib, lenvatinib, nintedanib, pazopanib, regorafenib, semaxanib, sorafenib, sunitinib, tivozanib, toceranib, or vandetanib, preferably suntinib, sorafenib, or pazopanib, more preferably sorafenib.

In preferred embodiments, the composition according to the invention or the miRNA from the composition is for use in the treatment of head and neck cancer (HNSCC). More preferably, the composition according to the invention or the miRNA from the composition is for use in the treatment of chemotherapy-resistant HNSCC such as HNSCC that is resistant to taxanes (for example cabazitaxel, docetaxel, larotaxel, ortataxel, paclitaxel, or tesetaxel, preferably paclitaxel or docetaxel), or that is resistant to pyrimidine-based antimetabolites (for example fluorouracil, capecitabine, doxifluridine, tegafur, carmofur, floxuridine, cytarabine, gemcitabine, azacitidine, or decitabine, preferably fluorouracil), or that is resistant to folic acid antimetabolites (aminopterin, methotrexate, pemetrexed, pralatrexate, or raltitrexed, preferably methotrexate), or that is resistant to platinum-based cell-cycle nonspecific antineoplastic agents (for example carboplatin, cisplatin, dicycloplatin, nedaplatin, oxaliplatin, or satraplatin, preferably cisplatin), or that is resistant to anthracyclins (for example aclarubicin, daunorubicin, doxorubicin, epirubicin, idarubicin, amrubicin, pirarubicin, valrubicin, or zorubicin, preferably doxorubicin), or that is resistant to intercalating crosslinking agents (for example actinomycin, bleomycin, mitomycins, plicamycin, preferably bleomycin or mitomycin).

In preferred embodiments, the composition according to the invention or the miRNA from the composition is for use in the treatment of prostate cancer. More preferably, the composition according to the invention or the miRNA from the composition is for use in the treatment of chemotherapy-resistant prostate cancer such as prostate cancer that is resistant to taxanes (for example cabazitaxel, docetaxel, larotaxel, ortataxel, paclitaxel, or tesetaxel, preferably docetaxel), or that is resistant to anthracenediones (for example mitoxantrone or pixantrone, preferably mitoxantrone), or that is resistant to alkylating antineoplastic agents (for example estrogen-based alkylating antineoplastic agents such as alestramustine, atrimustine, cytestrol acetate, estradiol mustard, estramustine, estromustine, stilbostat; or phenestrol, preferably estramustine).

In preferred embodiments, the composition according to the invention or the miRNA from the composition is for use in the treatment of tumours of the haematopoietic and lymphoid malignancies. More preferably, the composition according to the invention or the miRNA from the composition is for use in the treatment of chemotherapy-resistant tumours of the haematopoietic and lymphoid malignancies such as myeloma that is resistant to bortezomib, or that is resistant to lenalidomide, or such as lymphoma that is resistant to CHOP or to rituximab, such as resistance to cyclophosphamide or to anthracyclines such as hydroxydaunorubicin or to oncovin or to prednisone, or such as leukemia resistant to vincristine, anthracyclines such as doxorubicine, L-asparaginase, cyclophosphamide, methotrexate, 6-mercaptopurine, chlorambucil, cyclophosphamide, corticosteroids such as prednisone or prednisolone, fludarabine, pentostatin, or cladribine. Treatment of chemotherapy-resistant cancer such as sorafenib-resistant cancer as described herein can be as second line treatment when chemotherapy such as sorafenib treatment has been found to be ineffective, or to be less effective than anticipated or desired.

Solid tumors are often epithelial in origin (i.e. carcinomas). A loss of epithelial cell markers (e.g. E-cadherin) and gain of mesenchymal cell markers (e.g. N-cadherin and Vimentin) is known for patient tumor samples, including prostate cancer. Cancer cells can dedifferentiate through this so-called Epithelial to Mesenchymal Transition (EMT). During EMT, intercellular cell junctions are broken down, thereby giving tumor cells the ability to migrate and invade into the surrounding tissue or through blood vessel walls. Such phenotypic changes play a major role in dissemination of the disease and ultimately lead to disease progression, which is often associated with poor prognosis for the patients.

Loss of E-cadherin expression is considered as a molecular hallmark of EMT. EMT in tumor cells results from a transcriptional reprogramming of the cell. In particular the transcriptional repression of the E-cadherin (CDH1) gene promoter has been shown to trigger the EMT phenotype. The E-cadherin protein is one of the most important cadherin molecules mediating cell-cell contacts in epithelial cells/tissues. CDH1 is repressed by binding of the transcriptional repressors, SNAI1, SNAI2, TCF3, TWIST, ZEB1, ZEB2 or KLF8, to three so-called E-boxes in the CDH1 proximal promoter region. Inhibiting the binding of these repressors to the CDH1 promoter can revert EMT, also called mesenchymal to epithelial transition (MET), and inhibits tumor cell invasion and tumor progression.

In preferred embodiments, the composition according to the invention or the miRNA for the composition is for use in treatment, prevention, delay, or amelioration of a disease or a condition associated with EMT. Herein the miRNA is preferably a miRNA-518b molecule, miRNA-520f molecule, or a miRNA-524 molecule; or an isomiR or mimic thereof, or a precursor thereof. The disease or condition associated with EMT is preferably a cancer, more preferably a bladder or prostate cancer. This use is preferably by inducing a mesenchymal to epithelial transition.

In preferred embodiments, the composition according to the invention or the miRNA for the composition is for use in treatment, prevention, delay, or amelioration of cancer by downregulating the immunosuppressive tumour microenvironment. In related preferred embodiments, the composition according to the invention or the miRNA for the composition is for use in treatment, prevention, delay, or amelioration of cancer by preventing or reducing evasion of host immunity by a tumour. Such use is preferably for preventing, inhibiting, or reducing adenosine generation, for example by inhibiting or reducing activity of cell surface ectoenzymes such as those that dephosphorylate ATP to produce adenosine. Such use is more preferably for reducing NT5E expression and/or reducing ENTPD1 expression and/or inhibiting adenosine generation. More preferably, the composition according to the invention or the miRNA for the composition is for reducing NT5E expression; here, the miRNA for the composition is preferably miRNA-193a. More preferably, this composition according to the invention or this miRNA for the composition is for reducing ENTPD1 expression. More preferably, this composition according to the invention or this miRNA for the composition is for inhibiting adenosine generation. In even more preferred embodiments, this composition according to the invention or this miRNA for the composition is for reducing cancer cell migration, preferably for reducing adenosine-induced cancer cell migration, most preferably for reducing adenosine-induced cancer cell migration associated with NT5E expression. Reduction of NT5E or ENTPD1 expression is preferably assessed by luciferase assay or by RT-PCR, more preferably as described in the examples. Reduction of cancer cell migration is preferably assessed by in vitro transwell assays, more preferably as described in the examples.

In preferred embodiments, the composition according to the invention or the miRNA for the composition is for use in treatment, prevention, delay, or amelioration of cancer by promoting or increasing G2/M arrest in cancer cells, preferably in liver cancer cells, in lung cancer cells, in pancreatic cancer cells, in carcinoma cells, or in melanoma cells, more preferably in liver cancer cells, in carcinoma cells, or in melanoma cells, even more preferably in hepatocellular carcinoma cells or in melanoma cells.

Such use is preferably for reducing the expression or activity of factors that regulate cell division and/or proliferation by associating with the cytoskeleton, such as MPP2 and/or STMN1. Such use is preferably for promoting or increasing factors that bind and/or sequester cyclin-dependent kinases, such as YWHAZ and/or CCNA2. Preferably, the composition according to the invention or the miRNA for the composition is for use in treatment, prevention, delay, or amelioration of cancer by reducing the expression or activity of at least one of MPP2, STMN1, YWHAZ, and CCNA2, more preferably by reducing the expression or activity of at least YWHAZ or STMN1, even more preferably of at least YWHAZ, most preferably of each of MPP2, STMN1, YWHAZ, and CCNA2. Increase in G2/M arrest is preferably an increase as compared to untreated cells, and is preferably an increase of at least 5, 10, 15, 20, 25, 30, 35, 40, 45, or 50% or more. It is preferably assessed by DNA staining followed by microscopy imaging to determine nucleus intensity based on DNA content. Reduction of the expression or activity of at least one of MPP2, STMN1, YWHAZ, and CCNA2 is preferably assessed using RT-PCR, more preferably as described in the examples.

In preferred embodiments, the composition according to the invention or the miRNA for the composition is for use in treatment, prevention, delay, or amelioration of cancer by decreasing or reducing cancer cell migration, cancer cell adhesion, or cancer cell proliferation, or by increasing or promoting cancer cell apoptosis. These cancer cells are preferably lung cancer cells, liver cancer cells, breast cancer cells, melanoma cells, or carcinoma cells, more preferably lung cancer cells, liver cancer cells, breast cancer cells, or melanoma cells, even more preferably lung cancer cells such as A549 and H460, liver cancer cells such as Hep3B and Huh7, breast cancer cells such as BT549, and skin cancer cells such as A2058. In more preferred embodiments this use in treatment, prevention, delay, or amelioration of cancer is by decreasing expression or activity of at least one gene selected from the group consisting of FOXRED2, ERMP1, NT5E, SHMT2, HYOU1, TWISTNB, AP2M1, CLSTN1, TNFRSF21, DAZAP2, C1QBP, STARD7, ATP5SL, DCAF7, DHCR24, DPY19L1, AGPAT1, SLC30A7, AIMP2, UBP1, RUSC1, DCTN5, ATP5F1, CCDC28A, SLC35D2, WSB2, SEC61A1, MPP2, FAM60A, PITPNB, and POLE3, even more preferably from the group consisting of NT5E and TNFRSF21; preferably the use as described above for apoptosis, cell migration, adhesion, and proliferation is use for apoptosis, cell migration, adhesion, and/or proliferation associated with at least one of these genes. Expression is preferably assessed by RT-PCR, more preferably as described in the examples.

In preferred embodiments, the composition according to the invention or the miRNA for the composition is for use in treatment, prevention, delay, or amelioration of cancer by increasing or promoting apoptosis of cancer cells, preferably by increasing or promoting apoptosis associated with at least one gene selected from the group consisting of KCNMA1, NOTCH2, TNFRSF21, YWHAZ, CADM1, NOTCH1, CRYAA, ETS1, AIMP2, SQSTM1, ZMAT3, TGM2, CECR2, PDE3A, STRADB, NIPA1, MAPK8, TP53INP1, PRNP, PRT1, GCH1, DHCR24, TGFB2, NET1, PHLDA2, and TPP1, more preferably from the group consisting of NOTCH2, TNFRSF21, YWHAZ, ETS1, TGFB2, and MAPK8. Expression or activity of the gene is preferably reduced by the composition according to the invention or by the miRNA for the composition, such as by miRNA-193a.

In preferred embodiments, the composition according to the invention or the miRNA for the composition is for use in treatment, prevention, delay, or amelioration of cancer by decreasing or inhibiting angiogenesis, preferably angiogenesis associated with cancer cells, more preferably by decreasing or inhibiting angiogenesis associated with at least one gene selected from the group consisting of CRKL, CTGF, ZMIZ1, TGM2, ELK3, LOX, UBP1, PLAU, CYR61, and TGFB2, even more preferably CRKL, TGFB2 or PLAU, most preferably PLAU. Expression or activity of the gene is preferably reduced by the composition according to the invention or by the miRNA for the composition, such as by miRNA-193a.

In preferred embodiments, the composition according to the invention or the miRNA for the composition is for use in treatment, prevention, delay, or amelioration of cancer by modulating the unfolded protein response in cancer cells, more preferably by modulating the unfolded protein response associated with at least one gene selected from the group consisting of ERMP1, NCEH1, SEC31A, CLSTN1, FOXRED2, SEPN1, EXTL2, HYOU1, SLC35D1, SULF2, PTPLB, HHAT, ERAP2, FAF2, DPM3, PDZD2, SEC61A1, DHCR24, IDS, MOSPD2, DPM, PRNP, and AGPAT1. Expression or activity of the gene is preferably reduced by the composition according to the invention or by the miRNA for the composition, such as by miRNA-193a. Modulation of the unfolded protein response is preferably an inhibition or reduction of the unfolded protein response.

In preferred embodiments, the composition according to the invention or the miRNA for the composition is for use in treatment, prevention, delay, or amelioration of cancer by decreasing or inhibiting chemotaxis of cancer cells, more preferably by decreasing or inhibiting chemotaxis associated with at least one gene selected from the group consisting of CXCL1, RAC2, CXCL5, CYR61, PLAUR, KCNMA1, ABI2, and HPRT1, most preferably PLAUR. Expression or activity of the gene is preferably reduced by the composition according to the invention or by the miRNA for the composition, such as by miRNA-193a.

In preferred embodiments, the composition according to the invention or the miRNA for the composition is for use in treatment, prevention, delay, or amelioration of cancer by decreasing or inhibiting protein transport in cancer cells, more preferably by decreasing or inhibiting protein transport associated with at least one gene selected from the group consisting of STON2, RAB11FIP5, SRP54, YWHAZ, SYNRG, GCH1, THBS4, SRP54, TOMM20, SEC31A, TPP1, SLC30A7, TGFB2, AKAP12, AP2M1, ITGB3, GNAI3, SORL1, KRAS, SLC15A1, SEC61A1, APPL1, LRP4, PLEKHA8, STRADB, SCAMP4, HFE, CADM1, ZMAT3, ARF3, VAMP8, NUP50, DHCR24, RAB11FIP5, ATP6V1B2, SQSTM1, and WNK4, even more preferably YWHAZ, TGFB2, or KRAS, most preferably YWHAZ. Expression or activity of the gene is preferably reduced by the composition according to the invention or by the miRNA for the composition, such as by miRNA-193a.

In preferred embodiments, the composition according to the invention or the miRNA for the composition is for use in treatment, prevention, delay, or amelioration of cancer by decreasing or inhibiting nucleoside metabolism in cancer cells, more preferably by decreasing or inhibiting nucleoside metabolism associated with at least one gene selected from the group consisting of NUDT3, NUDT15, NUDT21, DERA, NT5E, GCH1, and HPRT1, most preferably NT5E. Expression or activity of the gene is preferably reduced by the composition according to the invention or by the miRNA for the composition, such as by miRNA-193a.

In preferred embodiments, the composition according to the invention or the miRNA for the composition is for use in treatment, prevention, delay, or amelioration of cancer by decreasing or inhibiting glycosylation of cancer cells, more preferably by decreasing or inhibiting glycosylation associated with at least one gene selected from the group consisting of SLC35D1, ST3GAL5, SULF2, LAT2, GALNT1, NCEH1, ST3GAL4, CHST14, B3GNT3, DPM3, GALNT13, DHCR24, NUDT15, IDH2, PPTC7, HPRT1, EXTL2, SEC61A1, ERAP2, and GALNT14. Expression or activity of the gene is preferably reduced by the composition according to the invention or by the miRNA for the composition, such as by miRNA-193a.

In preferred embodiments, the composition according to the invention or the miRNA for the composition is for use in treatment, prevention, delay, or amelioration of cancer by decreasing or inhibiting oncogenesis, more preferably by decreasing or inhibiting oncogenesis associated with at least one gene selected from the group consisting of CCND1, CBL, CXCL1, CRKL, MAX, KCNMA1, TBL1XR1, GNAI3, YWHAZ, RAC2, ETS1, PTCH1, MAPK8, LAMC2, PIK3R1, CDK6, CBL, APPL1, GNAI3, PDE3A, TGFB2, ABI2, MAX, ITGB3, LOX, CXCL5, ARPC5, PPARGC1A, and THBS4, even more preferably selected from CRKL, TGFB2, YWHAZ, ETS1, MAPK8, and CDK6, most preferably from YWHAZ, ETS1, MAPK8, and CDK6. Expression or activity of the gene is preferably reduced by the composition according to the invention or by the miRNA for the composition, such as by miRNA-193a.

In preferred embodiments, the composition according to the invention or the miRNA for the composition is for use in treatment, prevention, delay, or amelioration of cancer by decreasing or inhibiting dysfunctional wound healing, more preferably by decreasing or inhibiting dysfunctional wound healing associated with at least one gene selected from the group consisting of NOTCH2, KCNMA1, CXCL1, ITGB3, PLAU, CCND1, ZMIZ1, ELK3, YWHAZ, IL11, PLAUR, LOX, CTGF, and TGFB2, even more preferably selected from TGFB2, NOTCH2, PLAU, YWHAZ, and PLAUR, most preferably from NOTCH2, PLAU, YWHAZ, and optionally PLAUR. Expression or activity of the gene is preferably reduced by the composition according to the invention or by the miRNA for the composition, such as by miRNA-193a.

In preferred embodiments, the composition according to the invention or the miRNA for the composition is for use in treatment, prevention, delay, or amelioration of cancer by increasing or promoting immune activation, preferably immune activation associated with an immune response against cancer, more preferably by increasing or promoting immune activation associated with at least one gene selected from the group consisting of NOTCH2, LAT2, CRKL, LRRC8A, YWHAZ, PIK3R1, IRF1, TGFB2, IL11, UNG, CDK6, and HPRT1, even more preferably selected from CRKL, TGFB2, NOTCH2, YWHAZ, and CDK6, most preferably from NOTCH2, YWHAZ, and CDK6. Expression or activity of the gene is preferably reduced by the composition according to the invention or by the miRNA for the composition, such as by miRNA-193a.

The invention also provides a T-cell obtained from a subject treated with a miRNA for the composition or with a composition according to the invention, preferably with miRNA-193a or with a composition according to the invention comprising miRNA-193a. Such a T-cell can be for use in the treatment of cancer as described elsewhere herein. In its use, the T-cell is preferably previously obtained from a subject treated with a miRNA for the composition or with a composition according to the invention. The T-cell is preferably from a human subject. It is preferably for use as a vaccine, or for preventing recurrence or metastasis of cancer.

In preferred embodiments, the composition according to the invention or the miRNA for the composition, preferably miRNA-193a or a composition according to the invention comprising miRNA-193a, is for use in treatment, prevention, delay, or amelioration of a cancer associated with at least one gene selected from the group consisting of CDK6, EIF4B, ETS1, IL17RD, MCL1, MAPK8, NOTCH2, NT5E, PLAU, PLAUR, TNFRSF21, and YWHAZ, more preferably selected from NOTCH2, NT5E, PLAU, PLAUR, and YWHAZ.

In preferred embodiments, the composition according to the invention or the miRNA for the composition, preferably miRNA-193a or a composition according to the invention comprising miRNA-193a, is for use in treatment, prevention, delay, or amelioration of a cancer associated with at least one gene selected from the group consisting of CDK4, CDK6, CRKL, NT5E, HMGB1, IL17RD, KRAS, KIT, HDAC3, RTK2, TGFB2, TNFRSF21, PLAU, NOTCH1, NOTCH2, and YAP1. These genes have known involvement in anti-tumor immunity.

In preferred embodiments, the composition according to the invention or the miRNA for the composition, preferably miRNA-193a or a composition according to the invention comprising miRNA-193a, is for use in treatment, prevention, delay, or amelioration of a cancer associated with at least one gene selected from the group consisting of ETS1, YWHAZ, MPP2, PLAU, CDK4, CDK6, EIF4B, RAD51, CCNA2, STMN1, and DCAF7. These genes are involved in regulation of the cell cycle.

In preferred embodiments, the composition according to the invention or the miRNA for the composition, preferably miRNA-193a or a composition according to the invention comprising miRNA-193a, is for use in treatment, prevention, delay, or amelioration of cancer, wherein a preferred cancer is a cancer selected from the group consisting of colon cancer such as colon carcinoma, lung cancer such as lung carcinoma, melanoma, lymphoma such as reticulum cell sarcoma, pancreas cancer such as pancreatic adenocarcinoma, liver cancer such as hepatocarcinoma or hepatoma, breast cancer such as breast carcinoma, prostate cancer, kidney cancer such as renal adenocarcinoma, carcinoma such as adenocarcinoma or colon, lung, liver, pancreas, kidney, or breast carcinoma, and adenocarcinoma such as pancreatic or renal adenocarcinoma. A more preferred cancer is a cancer selected from the group consisting of colon cancer such as colon carcinoma, lung cancer such as lung carcinoma, melanoma, lymphoma such as reticulum cell sarcoma, pancreas cancer such as pancreatic adenocarcinoma, liver cancer such as hepatocarcinoma, breast cancer such as breast carcinoma, prostate cancer, carcinoma such as adenocarcinoma or colon, lung, liver, pancreas, or breast carcinoma, and adenocarcinoma such as pancreatic adenocarcinoma. An even more preferred cancer is a cancer selected from the group consisting of colon cancer such as colon carcinoma, lung cancer such as lung carcinoma, melanoma, lymphoma such as reticulum cell sarcoma, and carcinoma such as colon or lung carcinoma.

In further preferred embodiments, the composition according to the invention or the miRNA from the composition is for use in the treatment of cancer wherein the composition is combined with a further chemotherapeutic agent such as sorafenib. This is referred to hereinafter as a combination according to the invention. A combination according to the invention is preferably for use as described above for the composition for use according to the invention.

A combination according to the invention is a combination comprising a composition according to the invention or the miRNA from the composition and comprising a chemotherapeutic agent such as a kinase inhibitor drug suitable for the treatment of cancer, for example such as a combination comprising a composition according to the invention and comprising sorafenib, or for example comprising a miRNA from the composition and comprising sorafenib.

Suitable chemotherapeutic agents are kinase inhibitor drugs such as sorafenib or B-raf inhibitors or MEK inhibitors or RNR inhibitors or AURKB inhibitors. A preferred B-raf inhibitor is vemurafenib and/or dabrafenib. A preferred MEK inhibitor is trametinib and/or selumetinib. A preferred RNR inhibitor is selected from the group consisting of gemcitabine, hydroxyurea, clolar clofarabine and triapine

B-raf inhibitors are compounds that specifically inhibit the B-raf protein, for which a mutated form of the BRAF gene encodes. Several mutations of the BRAF gene are known to cause melanoma, and specific compounds have been developed which inhibit the mutated form of the B-raf protein. B-raf inhibitors are known in the art and include, but are not limited to vemurafenib, dabrafenib, trametinib, GDC-0879, PLX-4720, sorafenib, SB590885, PLX4720, XL281 and RAF265. B-raf inhibitors are e.g. described in Wong K. K., et al. One B-raf inhibitor may be used or together with other B-raf inhibitors in a combination according to the invention. Preferred B-raf inhibitors to be used in the present invention are vemurafenib, dabrafenib or a mixture of vemurafenib and dabrafenib. Vemurafenib is also known as RG7204 or N-(3-{[5-(4-chlorophenyl)-1H-pyrrolo[2,3-b]pyridin-3-yl]carbonyl}-2,4-difluorophenyl)propane-1-sulfonamide, and marketed as Zelboraf. Dabrafenib is also known as N-{3-[5-(2-aminopyrimidin-4-yl)-2-(1,1-dimethylethyl)thiazol-4-yl]-2-fluorophenyl}-2,6-difluorobenzenesulfonamide.

MEK inhibitors are compounds that specifically inhibit a MEK protein. Several MEK inhibitors are known in the art and include, but are not limited to trametinib (GSK1120212), selumetinib (AZD-6244), XL518, CI-1040, PD035901. Trametinib is also known as N-(3-(3-cyclopropyl-5-(2-fluoro-4-iodophenylamino)-6,8-dimethyl-2,4,7-trioxo-3,4,6,7-tetrahydropyrido[4,3-d]pyrimidin-1(2H)-yl)phenyl)acetamide. Selumetinib is also known as: 6-(4-bromo-2-chlorophenylamino)-7-fluoro-N-(2-hydroxyethoxy)-3-methyl-3Hbenzo[d]imidazole-5-carboxamide. MEK inhibitors are e.g. described in Wong, K. K. (PMID: 19149686). One MEK inhibitor may be used or together with other MEK inhibitors in a combination according to the invention. Several MEK inhibitors is synonymous with several distinct MEK inhibitors. Preferred MEK inhibitors to be used in the present invention are trametinib and/or selumetinib.

RNR and/or AURKB inhibitors are compounds that specifically inhibit RNR and/or AURKB proteins. RNR is a ribonucleotide reductase (RNR) and as such is the only enzyme responsible for the de novo conversion of ribonucleoside diphosphate (NDP) to deoxyribonucleoside diphosphate (dNDP) (Zhou et al. 2013). RNR is the key regulator of intracellular dNTP supply. Maintenance of a balanced dNTP pool is a fundamental cellular function because the consequences of imbalance in the substrates for DNA synthesis and repair include mutagenesis and cell death. Human RNR is composed of a subunits (RRM1) that contain the catalytic site and two binding sites for enzyme regulators and b subunits (RRM2) with a binuclear iron cofactor that generates the stable tyrosyl radical necessary for catalysis. An inhibitor of RNR may inhibit RRM1 and/or RRM2. Preferred RNR inhibitors are selected from the group consisting of gemcitabine, hydroxyurea, clolar clofarabine and triapine.

AURKB (Aurora B kinase) is a protein that functions in the attachment of the mitotic spindle to the centromere. Chromosomal segregation during mitosis as well as meiosis is regulated by kinases and phosphatases. The Aurora kinases associate with microtubules during chromosome movement and segregation. In cancerous cells, over-expression of these enzymes causes unequal distribution of genetic information, creating aneuploid cells, a hallmark of cancer.

A chemotherapeutic agent is a drug that is able to induce or promote an anti-cancer effect as defined herein. A preferred chemotherapeutic agent is a kinase inhibitor or an RNR inhibitor or an AURKB inhibitor. Examples of such inhibitors are compounds that specifically inhibit the RNR and/or the AURKB proteins. To evaluate the ability of a therapeutic compound to inhibit RNR and/or AURKB proteins, one can perform western blotting with RNR (RRM1 and/or RRM2) or AURKB protein as read-out. Cells are plated in 6-well plates and treated for 72 hours at 0.01, 0.1 and 1 uM of said compound. After treatment cells are scraped into a lysis buffer as a RIPA lysis buffer. Equal amounts of protein extracts are separated by using 10% SDS PAGE, and then transferred to a polyvinylidene difluoride membrane. After blocking for 1 hour in a Tris-buffered saline containing 0.1% Tween 20 and 5% nonfat milk, the membrane is probed with a RNR (i.e. RRM1 and/or RRM2) and/or a AURKB primary antibody, followed by a secondary antibody conjugated to horseradish peroxidase for chemiluminescent detection on film. Tubulin is used as loading control. A preferred RRM2 antibody used is from Santa Cruz (product #sc-10846) and/or a preferred AURKB antibody is from Cell Signalling (product #3094). The evaluation of the therapeutic ability of said RNR and/or AURKB inhibitor may also be assessed at the RNA level by carrying out a Northern blot or by PCR.

Preferred combinations according to the invention comprise:
i) a composition according to the invention or a miRNA from the composition, wherein the composition preferably comprises miRNA-193a or a mimic or isomiR or precursor thereof, or wherein the miRNA from the composition is miRNA-193a or a mimic or isomiR or precursor thereof, and
ii) at least one chemotherapeutic agent selected from the group consisting of
   a. receptor tyrosine kinase inhibitors such as VEGF receptor inhibitors, for example axitinib, cediranib, lenvatinib, nintedanib, pazopanib, regorafenib, semaxanib, sorafenib, sunitinib, tivozanib, toceranib, or vandetanib, preferably suntinib, sorafenib, or pazopanib, more preferably sorafenib;
   b. platinum-based cell-cycle nonspecific antineoplastic agents, for example carboplatin, cisplatin, dicycloplatin, nedaplatin, oxaliplatin, or satraplatin, preferably cisplatin or carboplatin or oxaliplatin;
   c. taxanes, for example cabazitaxel, docetaxel, larotaxel, ortataxel, paclitaxel, or tesetaxel, preferably paclitaxel or docetaxel, more preferably paclitaxel or docetaxel;
   d. pyrimidine-based antimetabolites, for example fluorouracil, capecitabine, doxifluridine, tegafur, carmofur, floxuridine, cytarabine, gemcitabine, azacitidine, or decitabine, preferably fluorouracil or gemcitabine or capecitabine;
   e. vinca alkaloids, for example vinblastine, vincristine, vinflunine, vindesine, or vinorelbine, preferably vinorelbine or vinblastine;
   f. folic acid antimetabolites, aminopterin, methotrexate, pemetrexed, pralatrexate, or raltitrexed, preferably pemetrexed or methotrexate;
   g. anthracyclins, for example aclarubicin, daunorubicin, doxorubicin, epirubicin, idarubicin, amrubicin, pirarubicin, valrubicin, or zorubicin, preferably to doxorubicin;
   h. nonclassical cell-cycle nonspecific antineoplastic agents, for example procarbazine, dacarbazine, temozolomide, altretamine, mitobronitol, or pipobroman, preferably dacarbazine or temozolomide;
   i. taxanes, for example cabazitaxel, docetaxel, larotaxel, ortataxel, paclitaxel, or tesetaxel, preferably paclitaxel such as albumin-bound paclitaxel;
   j. topoisomerase inhibitors, for example camptothecin, cositecan, belotecan, gimatecan, exatecan irinotecan, lurtotecan, silatecan, topotecan, rubitecan, preferably irinotecan;
   k. trifluridine or tipiracil, or a combination of trifluridine and tipiracil;
   l. intercalating crosslinking agents, for example actinomycin, bleomycin, mitomycins, plicamycin, preferably bleomycin or mitomycin;
   m. anthracenediones, for example mitoxantrone or pixantrone, preferably mitoxantrone; and
   n. alkylating antineoplastic agents, for example estrogen-based alkylating antineoplastic agents such as alestramustine, atrimustine, cytestrol acetate, estradiol mustard, estramustine, estromustine, stilbostat; or phenestrol, preferably estramustine.

In preferred embodiments, a composition according to the invention or a miRNA from the composition is for use in the treatment of cancer, wherein the composition increases the immune response to cancer cells. This may mean that it initiates an immune response in cases where no immune response was present.

In more preferred embodiments for increasing immune response, the composition according to the invention or a miRNA from the composition is for increasing the production of immune system activating cytokines, such as IL-2. Preferably, cytokine production is increased by 1%, 5%, 10%, 15%, 20%, 25%, 30%, 40%, 50%, 55%, 60%, 65%, 70% or 75%, or more, and is preferably detected through FACS, more preferably such as demonstrated in the examples. As demonstrated in the examples, immune system activating cytokines are increased in a 4T1 mouse model for triple negative breast cancer (TNBC) after one week of treatment. The increase in cytokines leads to increased immune suppression of cancers, and can lead to immune suppression or partial immune suppression of cancers that would otherwise not be susceptible to immune suppression. In preferred embodiments, the composition according to the invention or a miRNA from the composition is for increasing T-cell function, such as increasing production of IFNγ and IL-2.

In more preferred embodiments for increasing immune response, the composition according to the invention or a miRNA from the composition is for decreasing regulatory T cell population. Regulatory T cells (Tregs) are immunosuppressive T regulatory cells, and decreasing Tregs increases the immune response to a cancer. Preferably, Tregs are decreased by 1%; 5%, 10%, 15%, 20%, 25%, 30%, 40%, 50%, 55%, 60%, 65%, 70% or 75%, or more. Decrease of Tregs can be determined via the determination of FOXP3 or LAG3, for example as described in the examples. This effect is preferably in parallel with increased cytokine production as described above.

As demonstrated in the examples, recruitment of CD8+T effector cells is increased in a 4T1 mouse model for triple negative breast cancer (TNBC) after two weeks of treatment, and T-cell function is induced, while Treg population is decreased. Accordingly, in preferred embodiments for increasing immune response, the composition according to the invention or a miRNA from the composition is for increasing T-cell frequency. Preferably, such an increase is by 1%, 5%, 10%, 15%, 20%, 25%, 30%, 40%, 50%, 55%, 60%, 65%, 70% or 75%, or more. Such an increase can be determined by measuring CD8, for example as performed in the examples. In preferred embodiments for increasing immune response, the composition according to the invention or a miRNA from the composition is for inducing T-cell function, preferably for inducing T-cell function by inducing IFNγ production. Most preferably, the composition according to the invention or a miRNA from the composition is for increasing T-cell frequency and simultaneously inducing T-cell function, preferably while simultaneously decreasing regulatory T cell population. Tumors with decreased Tregs and with increased CD8+T effector cells are referred to as ‘hot’ tumors, which are tumors that do not have an immunosuppressed microenvironment. Conversely, tumors in an immunosuppressed microenvironment are referred to as ‘cold’ tumors.

Additionally, compositions according to the invention can reduce expression of immune suppressive target genes such as ENTPD1 (CD39) or TIM-3. Such a reduction is preferably by 1%, 5%, 10%, 15%, 20%, 25%, 30%, 40%, 50%, 55%, 60%, 65%, 70% or 75%, or more. TIM-3 or ENTPD1 expression can be determined via qPCR, for example as demonstrated in the examples. ENTPD1 is an ectonucleotidase that catalyses the hydrolysis of γ- and β-phosphate residues of triphospho- and diphosphonucleosides to the monophosphonucleoside derivative. It has an immune suppressive role through its generation of high amounts of adenosine. Reduction of ENTPD1 expression increases the immune response to tumor cells. TIM-3 is also known as hepatitis A virus cellular receptor 2 (HAVCR2), and is an immune checkpoint, an inhibitory receptor acting as an immune-suppressive marker. TIM-3 is mainly expressed on activated CD8+ T cells and suppresses macrophage activation. Reduction of TIM-3 expression increases the immune response to tumor cells. In preferred embodiments, the composition according to the invention or a miRNA from the composition is for reducing expression of ENTPD1 or of TIM-3 or for reducing expression of ENTPD1 and TIM-3.

The positive effect of compositions according to the invention and miRNA from the compositions on the immune system as it relates to tumor cells and cancer cells leads to the invention being suitable for preventing the growth of new tumors, preventing metastasis, or reducing the growth of tumors that have been removed in size, for example through surgery. For example, as demonstrated in example 4.4, treatment with a composition according to the invention reduced the regrowth of surgically excised tumors, and reduced metastasis of such tumors, increasing survival in affected subjects. A tumor from which metastases derive is referred to as a primary tumor. Moreover, subjects with a particular tumor type that had been treated with a composition according to the invention or with a miRNA from a composition show limited tumor take when re-challenged with new tumor cells of the same type that had already been treated. After the limited tumor take, the tumor fully regresses. When challenged with a different tumor type, the tumor fully takes, but also subsequently regresses entirely.

Accordingly, in preferred embodiments the compositions according to the invention and miRNA from the compositions are for use as a medicament for preventing, reducing, or delaying cancer or metastatic cancer. In this context, preferred cancers are breast cancer, carcinoma, and liver cancer, more preferably breast cancer and liver cancer.

Accordingly, in preferred embodiments the compositions according to the invention and miRNA from the compositions are for use as a cancer vaccine, preferably for use as a cancer vaccine for the prevention or treatment of cancer. Such vaccines are preferably for preventing or reducing regrowth or recurrence of primary tumors. Preferably, regrowth is reduced by 1%, 5%, 10%, 15%, 20%, 25%, 30%, 40%, 50%, 55%, 60%, 65%, 70% or 75%, or more. In another use, such vaccines are preferably for reducing or treating metastatic cancer. Preferably, metastatic cancer is reduced by 1%, 5%, 10%, 15%, 20%, 25%, 30%, 40%, 50%, 55%, 60%, 65%, 70% or 75%, or more, or motility of cancer cells is reduced by 1%, 5%, 10%, 15%, 20%, 25%, 30%, 40%, 50%, 55%, 60%, 65%, 70% or 75%, or more. In this context, preferred cancers are breast cancer, carcinoma, and liver cancer, more preferably breast cancer and liver cancer.

Accordingly, in preferred embodiments the compositions according to the invention and miRNA from the compositions are for use as a medicament, wherein the medicament is for the prevention, reduction, or treatment of metastatic cancer, preferably wherein the primary tumor has been surgically excised or has regressed, more preferably wherein the primary tumor has been surgically excised. Preferably, metastatic cancer is reduced by by 1%, 5%, 10%, 15%, 20%, 25%, 30%, 40%, 50%, 55%, 60%, 65%, 70% or 75%, or more. In this context, preferred cancers are breast cancer, carcinoma, and liver cancer, more preferably breast cancer and liver cancer.

Accordingly, in preferred embodiments the compositions according to the invention and miRNA from the compositions are for use as a medicament, wherein the medicament is for the prevention, reduction, or treatment of regrowth or recurrence of a cancer after surgical excision. Preferably, regrowth or recurrence is reduced by 1%, 5%, 10%, 15%, 20%, 25%, 30%, 40%, 50%, 55%, 60%, 65%, 70% or 75%, or more. In this context, preferred cancers are breast cancer, carcinoma, and liver cancer, more preferably breast cancer and liver cancer.

Accordingly, in preferred embodiments the compositions according to the invention and miRNA from the compositions are for use as a medicament, wherein the medicament is for the prevention, reduction, or treatment of regrowth or recurrence of a cancer after said cancer has regressed or has been successfully treated. Preferably, regrowth or recurrence is reduced by 1%, 5%, 10%, 15%, 20%, 25%, 30%, 40%, 50%, 55%, 60%, 65%, 70% or 75%, or more. In this context, preferred cancers are breast cancer, carcinoma, and liver cancer, more preferably breast cancer and liver cancer.

In preferred embodiments, the composition according to the invention or a miRNA from the composition is for inhibiting proliferation of tumour cells. As demonstrated in the examples, compositions according to the invention can reduce K-RAS and MCL1 expression, leading to a reduced proliferation of tumor cells. K-RAS, also known as KRAS, K-ras, Ki-ras, is a proto-oncogene known in the art. MCL1 is also known as induced myeloid leukaemia cell differentiation protein Mcl-1. It can enhance cancer cell survival by inhibiting apoptosis. Both K-RAS and MCL1 enhance proliferation of cancer cells. In preferred embodiments, the composition according to the invention or a miRNA from the composition is for reducing expression of K-RAS or of MCL1 or for reducing expression of K-RAS and MCL1. In preferred embodiments, the composition according to the invention or a miRNA from the composition is for reducing expression of K-RAS and MCL1 and ENTPD1 and TIM-3.

Inhibition of proliferation is preferably via induction of apoptosis. As demonstrated in the examples, compositions according to the invention induce apoptosis in cancer cells through caspase activation and PARP inactivation through PARP cleavage. Preferred caspase activation is activation of caspase 3/7. PARP is also known as poly (ADP-ribose) polymerase and refers to a family of proteins involved in programmed cell death. It is cleaved in vivo by caspase 3 and by caspase 7, which triggers apoptosis. Cleavage of PARP can be determined through blotting techniques, and caspase activation can be assayed by determining PARP cleavage through blotting, or by qPCR, for example as demonstrated in the examples. In preferred embodiments, the composition according to the invention or a miRNA from the composition is for inducing apoptosis in cancer cells. In preferred embodiments, the composition according to the invention or a miRNA from the composition is for activating caspase 3 and caspase 7. In preferred embodiments, the composition according to the invention or a miRNA from the composition is for inactivating PARP. Preferably, PARP is inactivated by 1%; 5%, 10%, 15%, 20%, 25%, 30%, 40%, 50%, 55%, 60%, 65%, 70% or 75%, or more. Inactivation of PARP can be monitored by blotting techniques as demonstrated in the examples, detecting the smaller fragments of the uncleaved enzyme. Preferably, caspase activity is increased by 1%; 5%, 10%, 15%, 20%, 25%, 30%, 40%, 50%, 55%, 60%, 65%, 70% or 75%, or more.

In further preferred embodiments, the composition according to the invention or a miRNA from the composition is for reducing expression of at least one of the genes selected from the group consisting of K-RAS, MCL1, ENTPD1, TIM-3, c-Kit, CyclinD1, and CD73. c-Kit is a proto-oncogene also known as tyrosine-protein kinase Kit or CD117, and codes for a receptor tyrosine kinase protein. Cyclin D1 overexpression correlates with early cancer onset and tumor progression. CD73 is also known as 5′-nucleotidase (5′-NT), and as ecto-5′-nucleotidase. The enzyme encoded by CD73 is ecto-5-prime-nucleotidase (5-prime-ribonucleotide phosphohydrolase; EC 3.1.3.5) and catalyzes the conversion at neutral pH of purine 5-prime mononucleotides to nucleosides, the preferred substrate being AMP.

Expression of such genes is preferably reduced by 1%, 5%, 10%, 15%, 20%, 25%, 30%, 40%, 50%, 55%, 60%, 65%, 70% or 75%, or more, which can for example be determined via qPCR techniques as demonstrated in the examples.

In preferred embodiments, the composition according to the invention or a miRNA from the composition is for regulating the adenosine A2A receptor pathway. The adenosine A2A receptor, also known as ADORA2A, is an adenosine receptor that can suppress immune cells. The activity of compositions according to the invention in reducing expression of CD73 and/or of ENTPD1, as described above, interferes with the A2A receptor pathway, reducing immune suppression. This leads to an anti-tumor effect because tumor cells ability to escape immune surveillance is reduced. In preferred embodiments, the composition according to the invention or a miRNA from the composition is for increasing the susceptibility of tumor cells to immune surveillance. Such an increase preferably leads to a reduction of tumor volume of 1%, 5%, 10%, 15%, 20%, 25%, 30%, 40%, 50%, 55%, 60%, 65%, 70% or 75%, or more. In more preferred embodiments, the composition according to the invention or a miRNA from the composition is for increasing the susceptibility of tumor cells to immune surveillance, while increasing recruitment of CD8+T effector cells, preferably while decreasing Tregs, such as through reducing expression of LAG3 or of FoxP3, or of both. Increased susceptibility to immune surveillance preferably leads to reduced tumor volume.

Compositions according to the invention and miRNA from the compositions promote cell cycle arrest in tumor cells. In preferred embodiments, compositions according to the invention or miRNA from the composition are for use in the treatment of cancer, wherein the use is for inducing cell cycle arrest. Cell cycle arrest profiles can be measured for example by performing either nuclei imaging or flow cytometry, preferably as demonstrated in the examples. In this context, cell cycle arrest is preferably the induction of a G2/M or a SubG1 cell cycle arrest profile. Preferably, 1%, 5%, 10%, 15%, 20%, 25%, 30%, 40%, 50%, 55%, 60%, 65%, 70% or 75%, or more tumor cells undergo cell cycle arrest. Preferably, when the composition according to the invention or the miRNA from the composition is for treating melanoma, liver cancer, carcinoma, lung cancer, or pancreas cancer, the composition according to the invention or the miRNA from the composition is for increasing cell cycle arrest profiles.

### General Definitions

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value more or less 1% of the value. When moieties or substructures of molecules are said to be identical, the natural abundance distribution of isotopes is not accounted for. The identical nature refers to a structural formula as it would be drawn.

As used herein, mol % refers to molar percentage, which is also known as a mole fraction or a molar fraction or a mole percent or an amount fraction. It relates to the amount in moles of a constituent, divided by the total amount of all constituents in a mixture, also expressed in moles.

When a structural formula or chemical name is understood by the skilled person to have chiral centers, yet no chirality is indicated, for each chiral center individual reference is made to all three of either the racemic mixture, the pure R enantiomer, and the pure S enantiomer.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components. It is understood that charge is often associated with equilibrium. A moiety that is said to carry or bear a charge is a moiety that will be found in a state where it bears or carries such a charge more often than that it does not bear or carry such a charge. As such, an atom that is indicated in this disclosure to be charged could be non-charged under specific conditions, and a neutral moiety could be charged under specific conditions, as is understood by a person skilled in the art.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use are suitable for use in methods of treatment.

The present invention has been described above with reference to a number of exemplary embodiments. Modifications and alternative implementations of some parts or elements are possible, and are included in the scope of protection as defined in the appended claims. All citations of literature and patent documents are hereby incorporated by reference.

### General Definitions and General Technologies Referred to Herein

MicroRNA molecules (“miRNAs”) are generally 21 to 22 nucleotides in length, though lengths of 17 and up to 25 nucleotides have been reported. Any length of 17, 18, 19, 20, 21, 22, 23, 24, 25 is therefore encompassed within the present invention. The miRNAs are each processed from a longer precursor RNA molecule (“precursor miRNA”). Precursor miRNAs are transcribed from non-protein-encoding genes. A precursor may have a length of at least 50, 70, 75, 80, 85, 100, 150, 200 nucleotides or more. The precursor miRNAs have two regions of complementarity that enables them to form a stem-loop- or fold-back-like structure, which is cleaved by enzymes called Dicer and Drosha in animals. Dicer and Drosha are ribonuclease III-like nucleases. The processed miRNA is typically a portion of the stem.

The processed miRNA (also referred to as “mature miRNA”) becomes part of a large complex, known as the RNA-Induced Silencing Complex (RISC) complex, to (down)-regulate a particular target gene. Examples of animal miRNAs include those that perfectly or imperfectly basepair with the mRNA target, resulting in either mRNA degradation or inhibition of translation respectively (Olsen et al, 1999; Seggerson et al, 2002). SiRNA molecules also are processed by Dicer, but from a long, double-stranded RNA molecule. SiRNAs are not naturally found in animal cells, but they can function in such cells in a RNA-induced silencing complex (RISC) to direct the sequence-specific cleavage of an mRNA target (Denli et al, 2003).

SIROCCO is a EU consortium which investigates silencing RNAs as organisers and coordinators of complexity in eukaryotic organisms (see for example the websites cordis.europa.eu/pub/lifescihealth/docs/sirocco.pdf and www.sirocco-project.eu). As a consortium, SIROCCO maintains a database of miRNA sequence information. Each miRNA entry listed in the SIROCCO database is based on observed and verified expression of said miRNA.

The study of endogenous miRNA molecules is described in U.S. Patent Application 60/575,743.

A miRNA is apparently active in the cell when the mature, single-stranded RNA is bound by a protein complex that regulates the translation of mRNAs that hybridize to the miRNA. Introducing exogenous RNA molecules that affect cells in the same way as endogenously expressed miRNAs requires that a single-stranded RNA molecule of the same sequence as the endogenous mature miRNA be taken up by the protein complex that facilitates translational control. A variety of RNA molecule designs have been evaluated. Three general designs that maximize uptake of the desired single-stranded miRNA by the miRNA pathway have been identified. An RNA molecule with a miRNA sequence having at least one of the three designs may be referred to as a synthetic miRNA.

miRNA molecules of the invention can replace or supplement the gene silencing activity of an endogenous miRNA. An example of such molecules, preferred characteristics and modifications of such molecules and compositions comprising such molecules is described in WO2009/091982.

miRNA molecules of the invention or isomiRs or mimics or sources thereof comprise, in some embodiments, two RNA molecules wherein one RNA is identical to a naturally occurring, mature miRNA. The RNA molecule that is identical to a mature miRNA is referred to as the active strand or the antisense strand. The second RNA molecule, referred to as the complementary strand or the sense strand, is at least partially complementary to the active strand. The active and complementary strands are hybridized to create a double-stranded RNA, that is similar to the naturally occurring miRNA precursor that is bound by the protein complex immediately prior to miRNA activation in the cell. Maximizing activity of said miRNA requires maximizing uptake of the active strand and minimizing uptake of the complementary strand by the miRNA protein complex that regulates gene expression at the level of translation. The molecular designs that provide optimal miRNA activity involve modifications of the complementary strand. Two designs incorporate chemical modifications of the complementary strand. The first modification involves creating a complementary RNA with a group other than a phosphate or hydroxyl at its 5′ terminus. The presence of the 5′ modification apparently eliminates uptake of the complementary strand and subsequently favors uptake of the active strand by the miRNA protein complex. The 5′ modification can be any of a variety of molecules including NH2, NHCOCH3, biotin, and others. The second chemical modification strategy that significantly reduces uptake of the complementary strand by the miRNA pathway is incorporating nucleotides with sugar modifications in the first 2-6 nucleotides of the complementary strand. It should be noted that the sugar modifications consistent with the second design strategy can be coupled with 5′ terminal modifications consistent with the first design strategy to further enhance miRNA activities. The third miRNA design involves incorporating nucleotides in the 3′ end of the complementary strand that are not complementary to the active strand. Hybrids of the resulting active and complementary RNAs are very stable at the 3′ end of the active strand but relatively unstable at the 5′ end of the active strand. Studies with siRNAs indicate that 5′ hybrid stability is a key indicator of RNA uptake by the protein complex that supports RNA interference, which is at least related to the miRNA pathway in cells. The inventors have found that the judicious use of mismatches in the complementary RNA strand significantly enhances the activity of said miRNA.

### Nucleic Acids

The present invention concerns nucleic acid molecules also called sources or precursors of miRNAs that can introduce miRNAs in cultured cells or into a subject. The nucleic acids may have been produced in cells or in vitro by purified enzymes though they are preferentially produced by chemical synthesis. They may be crude or purified. The term “miRNA,” unless otherwise indicated, refers to the processed miRNA, after it has been cleaved from its precursor. The name of the miRNA is often abbreviated and referred to without the prefix and will be understood as such, depending on the context. Unless otherwise indicated, miRNAs referred to in the application are human sequences identified as mir-X or let-X, where X is a number and/or letter.

It is understood that a miRNA is derived from genomic sequences or a non-coding gene. In this respect, the term “gene” is used for simplicity to refer to the genomic sequence encoding the precursor miRNA for a given miRNA. However, embodiments of the invention may involve genomic sequences of a miRNA that are involved in its expression, such as a promoter or other regulatory sequences.

The term “recombinant” may be used and this generally refers to a molecule that has been manipulated in vitro or that is the replicated or expressed product of such a molecule.

The term “nucleic acid” is well known in the art. A “nucleic acid” as used herein will generally refer to a molecule (one or more strands) of DNA, RNA or a derivative or analogue thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.”

The term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complement(s)” of a particular sequence comprising a molecule. For example, precursor miRNA may have a self-complementary region, which is up to 100% complementary.

As used herein, “hybridization”, “hybridizes” or “capable of hybridizing” is understood to mean the forming of a double or triple stranded molecule or a molecule with partial double or triple stranded nature using techniques known to the skilled person such as southern blotting procedures. The term “anneal” as used herein is synonymous with “hybridize.” The term “hybridization”, “hybridize(s)” or “capable of hybridizing” may mean “low”, “medium” or “high” hybridization conditions as defined below.

Low to medium to high stringency conditions means prehybridization and hybridization at 42° C. in 5×SSPE, 0.3% SDS, 200 μg/ml sheared and denatured salmon sperm DNA, and either 25% 35% or 50% formamide for low to medium to high stringencies respectively. Subsequently, the hybridization reaction is washed three times for 30 minutes each using 2×SSC, 0.2% SDS and either 55° C., 65° C., or 75° C. for low to medium to high stringencies.

Nucleic acids or derivatives thereof of the invention will comprise, in some embodiments the miRNA sequence of any miRNA described in SEQ ID NOs: 51-125. It is contemplated that nucleic acids sequences of the invention derived from SEQ ID NO: 51-125 can have, have at least, or have at most 5, 6, 7, 8, 9, 10, 11, 12, 13, 14, 15, 16, 17, 18, 19, 20, 21, 22, 23, contiguous nucleotides from SEQ ID NOs: 51-125 (or any range derivable therein). In other embodiments, nucleic acids are, are at least, or are at most 80, 81, 82, 83, 84, 85, 86, 87, 88, 89, 90, 91, 92, 93, 94, 95, 96, 97, 98, 99, 100% identical to the miRNA sequence of SEQ ID NOs: 51-125.

### Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in a manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, carboxyalkyl, amino, hydroxyl, halogen (i.e., fluoro, chloro, bromo, or iodo), thiol or alkylthiol moeity. Preferred alkyl (e.g., alkyl, carboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. Other examples are well known to those of skill in the art.

A nucleobase may be comprised in a nucleoside or nucleotide, using any chemical or natural synthesis method described herein or known to one of ordinary skill in the art. Such nucleobase may be labeled or it may be part of a molecule that is labeled and contains the nucleobase.

### Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the l′-position of a 5-carbon sugar. In another non-limiting example, a nucleoside comprising a pyrimidine nucleobase (i.e., C, T or U) typically covalently attaches a 1 position of a pyrimidine to a l′-position of a 5-carbon sugar (Kornberg and Baker, 1992).

### Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

### Nucleic Acid Analogs

A nucleic acid may comprise, or be composed entirely of, a derivative or analogue of a nucleobase, a nucleobase linker moiety and/or backbone moiety that may be present in a naturally occurring nucleic acid. RNA with nucleic acid analogues may also be labeled according to methods of the invention. As used herein a “derivative” refers to a chemically modified or altered form of a naturally occurring molecule, while the terms “mimic” or “analogue” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980).

Additional non-limiting examples of nucleosides, nucleotides or nucleic acids comprising 5-carbon sugar and/or backbone moiety derivatives or analogs, include those in: U.S. Pat. No. 5,681,947, which describes oligonucleotides comprising purine derivatives that form triple helixes with and/or prevent expression of dsDNA; U.S. Pat. Nos. 5,652,099 and 5,763,167, which describe nucleic acids incorporating fluorescent analogs of nucleosides found in DNA or RNA, particularly for use as fluorescent nucleic acids probes; U.S. Pat. No. 5,614,617, which describes oligonucleotide analogs with substitutions on pyrimidine rings that possess enhanced nuclease stability; U.S. Pat. Nos. 5,670,663, 5,872,232 and 5,859,221, which describe oligonucleotide analogs with modified 5-carbon sugars (i.e., modified T-deoxyfuranosyl moieties) used in nucleic acid detection; U.S. Pat. No. 5,446,137, which describes oligonucleotides comprising at least one 5-carbon sugar moiety substituted at the 4′ position with a substituent other than hydrogen that can be used in hybridization assays; U.S. Pat. No. 5,886,165, which describes oligonucleotides with both deoxyribonucleotides with 3′-5′ internucleotide linkages and ribonucleotides with 2′-5′ internucleotide linkages; U.S. Pat. No. 5,714,606, which describes a modified internucleotide linkage wherein a 3′-position oxygen of the internucleotide linkage is replaced by a carbon to enhance the nuclease resistance of nucleic acids; U.S. Pat. No. 5,672,697, which describes oligonucleotides containing one or more 5′ methylene phosphonate internucleotide linkages that enhance nuclease resistance; U.S. Pat. Nos. 5,466,786 and 5,792,847, which describe the linkage of a substituent moiety which may comprise a drug or label to the 2′ carbon of an oligonucleotide to provide enhanced nuclease stability and ability to deliver drugs or detection moieties; U.S. Pat. No. 5,223,618, which describes oligonucleotide analogs with a 2′ or 3′ carbon backbone linkage attaching the 4′ position and 3′ position of adjacent 5-carbon sugar moiety to enhanced cellular uptake, resistance to nucleases and hybridization to target RNA; U.S. Pat. No. 5,470,967, which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240, which describe oligonucleotides with three or four atom linker moiety replacing phosphodiester backbone moiety used for improved nuclease resistance, cellular uptake and regulating RNA expression; U.S. Pat. No. 5,858,988, which describes hydrophobic carrier agent attached to the 2′-0 position of oligonucleotides to enhanced their membrane permeability and stability; U.S. Pat. No. 5,214,136, which describes oligonucleotides conjugated to anthraquinone at the 5′ terminus that possess enhanced hybridization to DNA or RNA; enhanced stability to nucleases; U.S. Pat. No. 5,700,922, which describes PNA-DNA-PNA chimeras wherein the DNA comprises 2′-deoxy-erythro-pentofuranosyl nucleotides for enhanced nuclease resistance, binding affinity, and ability to activate RNase H; and WO98/39352, WO99/14226, WO2003/95467 and WO2007/085485, which describe modified RNA nucleotides of which the ribose moiety is modified with an extra bridge connecting the 2′ oxygen and 4′ carbon. The locked ribose significantly increases the binding affinity and specificity; and WO2008/147824, which describes modified RNA nucleotides termed UNA (unlocked nucleic acid). UNA are acyclic analogues of RNA in which the bond between the C2′ and C3′ atoms has been cleaved, decreasing binding affinity towards a complementary strand. UNA are compatible with RNase H recognition and RNA cleavage and improves siRNA mediated gene silencing; WO2008/036127 which describes Morpholino nucleic acid analogues, which contain both uncharged and cationic intersubunit linkages; WO/2007/069092 and EP2075342 which describe Zip Nucleic Acids (ZNA), containing conjugating spermine derivatives as cationic moieties (Z units) to an oligonucleotide; U.S. Pat. No. 5,708,154, which describes RNA linked to a DNA to form a DNA-RNA hybrid; U.S. Pat. No. 5,728,525, which describes the labeling of nucleoside analogs with a universal fluorescent label.

Additional teachings for nucleoside analogs and nucleic acid analogs are U.S. Pat. No. 5,728,525, which describes nucleoside analogs that are end-labeled; U.S. Pat. Nos. 5,637,683, 6,251,666 (L-nucleotide substitutions), and 5,480,980 (7-deaza-2′-deoxyguanosine nucleotides and nucleic acid analogs thereof). The use of other analogs is specifically contemplated for use in the context of the present invention. Such analogs may be used in synthetic nucleic acid molecules of the invention, both throughout the molecule or at selected nucleotides. They include, but are not limited to,

1) ribose modifications (such as 2′F, 2′ NH2, 2′N3,4′thio, or 2′ O—CH3) and
2) phosphate modifications (such as those found in phosphorothioates, methyl phosphonates, and phosphoroborates).

Such analogs have been created to confer stability on RNAs by reducing or eliminating their capacity to be cleaved by ribonucleases. When these nucleotide analogs are present in RNAs, they can have profoundly positive effects on the stability of the RNAs in animals. It is contemplated that the use of nucleotide analogs can be used alone or in conjunction with any of the design modifications of a synthetic miRNA for any nucleic acid of the invention.

### Modified Nucleotides

miRNAs of the invention specifically contemplate the use of nucleotides that are modified to enhance their activities. Such nucleotides include those that are at the 5′ or 3′ terminus of the RNA as well as those that are internal within the molecule. Modified nucleotides used in the complementary strands of said miRNAs either block the 5′OH or phosphate of the RNA or introduce internal sugar modifications that enhance uptake of the active strand of the miRNA. Modifications for the miRNAs include internal sugar modifications that enhance hybridization as well as stabilize the molecules in cells and terminal modifications that further stabilize the nucleic acids in cells. Further contemplated are modifications that can be detected by microscopy or other methods to identify cells that contain the synthetic miRNAs.

### Preparation of Nucleic Acids

A nucleic acid may be made by any technique known to one of ordinary skill in the art, such as for example, chemical synthesis, enzymatic production or biological production.

Design of miRNAs

miRNAs typically comprise two strands, an active strand that is identical in sequence to the mature miRNA that is being studied and a complementary strand that is at least partially complementary to the active strand. The active strand is the biologically relevant molecule and should be preferentially taken up by the complex in cells that modulates translation either through mRNA degradation or translational control. Preferential uptake of the active strand has two profound results: (1) the observed activity of said miRNA increases dramatically and (2) non-intended effects induced by uptake and activation of the complementary strand are essentially eliminated. According to the invention, several miRNA designs can be used to ensure the preferential uptake of the active strand.

### 5′ Blocking Agent

The introduction of a stable moiety other than phosphate or hydroxyl at the 5′ end of the complementary strand impairs its activity in the miRNA pathway. This ensures that only the active strand of the miRNA will be used to regulate translation in the cell. 5′ modifications include, but are not limited to, NH2, biotin, an amine group, a lower alkylamine group, an acetyl group, 2′ O-Me, DMTO, fluoroscein, a thiol, or acridine or any other group with this type of functionality.

Other sense strand modifications. The introduction of nucleotide modifications like 2′-O Me, 2′-deoxy, T-deoxy-2′-fluoro, 2′-O-methyl, 2′-O-methoxyethyl (2′-0-MOE), 2′-O-aminopropyl (2′-0-AP), 2′-O-dimethylaminoethyl (2′-0-DMAOE), 2′-O-dimethylaminopropyl (2′-0-DMAP), 2′-O-dimethylaminoethyloxyethyl (2′-0-DMAEOE), or 2′-O—N-methylacetamido (2′-0-NMA), NH2, biotin, an amine group, a lower alkylamine group, an acetyl group, DMTO, fluoroscein, a thiol, or acridine or any other group with this type of functionality in the complementary strand of the miRNA can eliminate the activity of the complementary strand and enhance uptake of the active strand of the miRNA.

Base mismatches in the sense strand. As with siRNAs (Schwarz 2003), the relative stability of the 5′ and 3′ ends of the active strand of the miRNA apparently determines the uptake and activation of the active by the miRNA pathway. Destabilizing the 5′ end of the active strand of the miRNA by the strategic placement of base mismatches in the 3′ end of the complementary strand of the synthetic miRNA enhances the activity of the active strand and essentially eliminates the activity of the complementary strand.

### Host Cells and Target Cells

The cells wherein a miRNA or source thereof is introduced or wherein the presence of a miRNA is assessed may be derived from or contained in any organism. Preferably, the cell is a vertebrate cell. More preferably, the cell is a mammalian cell. Even more preferably, the cell is a human cell.

A mammalian cell may be from the germ line or somatic, totipotent or pluripotent, dividing or non-dividing, epithelium, immortalized or transformed, or the like. The cell may be an undifferentiated cell, such as a stem cell, or a differentiated cell, such as from a cell of an organ or tissue. Alternatively, cells may be qualified as epithelial or endothelial cells, stromal cells, brain, breast, cervix, colon, gastrointestinal tract, heart, kidney, large intestine, liver, lung, ovary, pancreas, heart, prostate, bladder, small intestine, stomach, testes or uterus.

As used herein, the terms “cell,” “cell line,” and “cell culture” may be used interchangeably. All of these terms also include their progeny, which is any and all subsequent generations formed by cell division. It is understood that all progeny may not be identical due to deliberate or inadvertent mutations. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a small, interfering RNA or a template construct encoding a reporter gene has been introduced. Therefore, recombinant cells are distinguishable from naturally occurring cells that do not contain a recombinantly introduced nucleic acid.

A tissue may comprise a host cell or cells to be transformed or contacted with a nucleic acid delivery composition and/or an additional agent. The tissue may be part or separated from an organism. In certain embodiments, a tissue and its constituent cells may comprise, but is not limited to brain, cerbellum, spinal cord, brachial nerve, intercostal nerves, musculocultaneous nerve, subcostal nerve, lumbar plexus, sacral plexus, femoral nerve, pudental nerve, sciatic nerve, muscular brenches of femoral nerve, saphnous nerve, tibial nerve, radial nerve, median nerve, iliophypogastric nerve, genitofemoral nerve, obturator nerve, ulnar nerve, common peroneal nerve, deep pernneal nerve, superficial peroneal nerve, ganglion, optic nerve, nerve cells, stem cells.

In certain embodiments, the host cell or tissue may be comprised in at least one organism. In certain embodiments, the organism may be a mammal, a human, a primate or murine. One of skill in the art would further understand the conditions under which to incubate all of the above described host cells to maintain them and to permit their division to form progeny.

### Delivery Methods

RNA molecules may be encoded by a nucleic acid molecule comprised in a vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, lentivirus, and plant viruses), and artificial chromosomes (e.g., YACs). One of skill in the art would be well equipped to construct a vector through standard recombinant techniques, which are described in Sambrook et al, 1989 and Ausubel et al, 1996, both incorporated herein by reference. In addition to encoding a modified polypeptide such as modified gelonin, a vector may encode non-modified polypeptide sequences such as a tag or targeting molecule. A targeting molecule is one that directs the desired nucleic acid to a particular organ, tissue, cell, or other location in a subject's body.

The term “expression vector” refers to a vector containing a nucleic acid sequence coding for at least part of a gene product capable of being transcribed. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described. Such vectors may be encapsulated in lipid nanoparticles according to the invention.

### Nanoparticle Functionalisation

A variety of compounds have been attached to the periphery of nanoparticles to facilitate their transport across cell membranes. Short signal peptides found in the HIV TAT, HSV VP22, Drosphila antennapedia, and other proteins have been found to enable the rapid transfer of biomolecules across membranes (reviewed by Schwarze 2000). These signal peptides, referred to as Protein Transduction Domains (PTDs), have been attached to oligonucleotides to facilitate their delivery into cultured cells (Eguchi A, Dowdy S F, Trends Pharmacol Sci., 2009, 7:341-5). Likewise, poly-L-lysine has been conjugated to oligonucleotides to decrease the net negative charge and improve uptake into cells (Leonetti 1990). Various signal peptides or ligands or transduction peptides or ligands can be linked to the surface of a nanoparticle according to the invention, for example by conjugating the peptides or ligands to a lipophilic anchor as defined earlier herein.

### Therapeutic Applications

miRNAs that affect phenotypic traits provide intervention points for therapeutic applications as well as diagnostic applications (by screening for the presence or absence of a particular miRNA). It is specifically contemplated that RNA molecules of the present invention can be used to treat any of the diseases or conditions discussed in the previous section. Moreover, any of the methods described above can also be employed with respect to therapeutic and diagnostic aspects of the invention. For example, methods with respect to detecting miRNAs or screening for them can also be employed in a diagnostic context. In therapeutic applications, an effective amount of the miRNAs of the present invention is administered to a cell, which may or may not be in an animal. In some embodiments, a therapeutically effective amount of the miRNAs of the present invention is administered to an individual for the treatment of disease or condition. The term “effective amount” as used herein is defined as the amount of the molecules of the present invention that are necessary to result in the desired physiological change in the cell or tissue to which it is administered. The term “therapeutically effective amount” as used herein is defined as the amount of the molecules of the present invention that achieves a desired effect with respect to a disease or condition associated with neo-angiogenesis as earlier defined herein. A skilled artisan readily recognizes that in many cases the molecules may not provide a cure but may provide a partial benefit, such as alleviation or improvement of at least one symptom. In some embodiments, a physiological change having some benefit is also considered therapeutically beneficial. Thus, in some embodiments, an amount of molecules that provides a physiological change is considered an “effective amount” or a “therapeutically effective amount.”

In certain embodiments, pharmaceutical compositions may comprise, for example, at least about 0.1% of an active compound. In other embodiments, an active compound may comprise 2% to 75% of the weight of the unit, or 25% to 60%, for example, and any range derivable therein. In other non-limiting examples, a dose may also comprise less than 1 microgram/kg/body weight, or 1 microgram/kg/body weight, from 5 microgram/kg/body weight, 10 microgram/kg/body weight, 50 microgram/kg/body weight, 100 microgram/kg/body weight, 200 microgram/kg/body weight, 350 microgram/kg/body weight, 500 microgram/kg/body weight, 1 milligram/kg/body weight, 5 milligram/kg/body weight, 10 milligram/kg/body weight, 50 milligram/kg/body weight, 100 milligram/kg/body weight, 200 milligram/kg/body weight, 350 milligram/kg/body weight, or 500 milligram/kg/body weight, to 1000 mg/kg/body weight or more per administration, and any range derivable therein. In non-limiting examples of a derivable range from the numbers listed herein, a range of 5 mg/kg/body weight to 100 mg/kg/body weight, 5 microgram/kg/body weight to 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens, chlorobutanol, phenol, sorbic acid, thimerosal or combinations thereof.

The molecules may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

The composition is generally a suspension of nanoparticles in an aqueous medium. However, it can be lyophilized and provided as a powder, wherein the powder comprises the nanoparticles and optionally buffer salts or other excipients.

### Effective Dosages

The molecules of the invention will generally be used in an amount effective to achieve the intended purpose. For use to treat or prevent a disease condition, the molecules of the invention, or pharmaceutical compositions thereof, are administered or applied in a therapeutically effective amount. A therapeutically effective amount is an amount effective to ameliorate or prevent the symptoms, or prolong the survival of the patient being treated. Determination of a therapeutically effective amount is well within the capabilities of those skilled in the art, especially in light of the detailed disclosure provided herein. For systemic administration, a therapeutically effective dose can be estimated initially from in vitro assays. For example, a dose can be formulated in animal models to achieve a circulating concentration range that includes the EC50 as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data. Dosage amount and interval may be adjusted individually to provide plasma levels of the molecules which are sufficient to maintain therapeutic effect. Usual patient dosages for administration by injection range from 0.01 to 0.1 mg/kg/day, or from 0.1 to 5 mg/kg/day, preferably from 0.5 to 1 mg/kg/day or more. Therapeutically effective serum levels may be achieved by administering multiple doses each day.

In cases of local administration or selective uptake, the effective local concentration of the proteins may not be related to plasma concentration. One having skill in the art will be able to optimize therapeutically effective local dosages without undue experimentation. The amount of molecules administered will, of course, be dependent on the subject being treated, on the subject's weight, the severity of the affliction, the manner of administration and the judgment of the prescribing physician. The therapy may be repeated intermittently while symptoms detectable or even when they are not detectable. The therapy may be provided alone or in combination with other drugs or treatment (including surgery).

### Kits

Any of the compositions described herein may be comprised in a kit. In a non-limiting example, individual miRNAs are included in a kit, also comprising diamino lipid. The kit may further include one or more negative control synthetic miRNAs that can be used to control for the effects of synthetic miRNA delivery. The kit may further include water and hybridization buffer to facilitate hybridization of the two strands of the synthetic miRNAs. The kit may also include one or more transfection reagent(s) to facilitate delivery of the miRNA to cells.

### Sequence Identity

“Sequence identity” is herein defined as a relationship between two or more nucleic acid (nucleotide, polynucleotide, RNA, DNA) sequences, as determined by comparing the sequences. In the art, “identity” also means the degree of sequence relatedness between nucleic acid sequences, as the case may be, as determined by the match between strings of such sequences. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988). In an embodiment, identity is assessed on a whole length of a given SEQ ID NO.

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Chemotherapeutic Agents

Examples of chemotherapeutic agents for use in combinations according to the invention include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma and calicheamicin omega); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-II); topoisomerase inhibitor RFS 2000; difluorometlhylornithine (DMFO); retinoids such as retinoic acid; capecitabine; gefitinib and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LYI 17018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in aberrant cell proliferation, such as, for example, PKC-α, Raf and H-Ras; ribozymes such as a VEGF expression inhibitor and a HER2 expression inhibitor; vaccines such as gene therapy vaccines and pharmaceutically acceptable salts, acids or derivatives of any of the above. A list of U.S. FDA approved oncology drags with their approved indications can be found on the World Wide Web at accessdata.fda.gov/scripts/cder/onctools/druglist.cfm. A suitable RNR inhibitor is selected from the group consisting of gemcitabine, hydroxyurea, clolar, clofarabine, and triapine. A suitable AURKB inhibitor is selected from the group consisting of: AZD1152, VX-680, MLN8054, MLN8237, PHA680632, PH739358, Hesperidin, ZM447439, JNJ770621, SU6668, CCT129202, AT9283, MP529, SNS314, R763, ENMD2076, XL228, TTP687, PF03814735 and CYC116. Another suitable anticancer drug is gefitinib.

Moreover, it is contemplated that samples that have differences in the activity of certain pathways may also be compared. Such cellular pathways include but are not limited to the following: any adhesion or motility pathway including but not limited to those involving cyclic AMP, protein kinase A, G-protein couple receptors, adenylyl cyclase, L-selectin, E-selectin, PECAM, VCAM-I, α-actinin, paxillin, cadherins, AKT, integrin-α, integrin-β, RAF-I, ERK, PI-3 kinase, vinculin, matrix metalloproteinases, Rho GTPases, p85, trefoil factors, profilin, FAK, MAP kinase, Ras, caveolin, calpain-1, calpain-2, epidermal growth factor receptor, ICAM-1, ICAM-2, cofilin, actin, gelsolin, Rho A, Rac, myosin light chain kinase, platelet-derived growth factor receptor or ezrin; any apoptosis pathway including but not limited to those involving AKT, Fas ligand, NFKB, caspase-9, PB kinase, caspase-3, caspase-7, ICAD, CAD, EndoG, Granzyme B, Bad, Bax, Bid, Bak, APAF-I, cytochrome C, p53, ATM, BcI-2, PARP, Chkl, Chk2, Rho-21, c-Jun, Rho73, Rad51, Mdm2, Rad50, c-Abl, BRCA-I, perforin, caspase-4, caspase-8, caspase-6, caspase-1, caspase-2, caspase-10, Rho, Jun kinase, Jun kinase kinase, Rip2, lannin-A, lannin-BI, lannin-B2, Fas receptor, H2O2, Granzyme A, NADPH oxidase, HMG2, CD4, CD28, CD3, TRADD, IKK, FADD, GADD45, DR3 death receptor, DR4/5 death receptor, FLIPs, APO-3, GRB2, SHC, ERK, MEK, RAF-1, cyclic AMP, protein kinase A, E2F, retinoblastoma protein, Smac/Diablo, ACH receptor, 14-3-3, FAK, SODD, TNF receptor, RTP, cyclin-DI, PCNA, Bcl-XL, PIP2, PIP3, PTEN, ATM, Cdc2, protein kinase C, calcineurin, IKKα, IKKβ, IKKγ, SOS-I, c-FOS, Traf-1, Traf-2, IκBβ or the proteasome; any cell activation pathway including but not limited to those involving protein kinase A, nitric oxide, caveolin-1, actin, calcium, protein kinase C, Cdc2, cyclin B, Cdc25, GRB2, SRC protein kinase, ADP-ribosylation factors (ARFs), phospholipase D, AKAP95, p68, Aurora B, CDKI, Eg7, histone H3, PKAc, CD80, PI3 kinase, WASP, Arp2, Arp3, p34, p20, PP2A, angiotensin, angiotensin-converting enzyme, protease-activated receptor-1, protease-activated receptor-4, Ras, RAF-I, PLCβ, PLCγ, COX-I, G-protein-coupled receptors, phospholipase A2, IP3, SUMOI, SUMO 2/3, ubiquitin, Ran, Ran-GAP, Ran-GEF, p53, glucocorticoids, glucocorticoid receptor, components of the SWI/SNF complex, RanBPI, RanBP2, importins, exportins, RCCI, CD40, CD40 ligand, p38, DCKα, IKKβ, NFKB, TRAF2, TRAF3, TRAF5, TRAF6, IL-4, IL-4 receptor, CDK5, AP-I transcription factor, CD45, CD4, T cell receptors, MAP kinase, nerve growth factor, nerve growth factor receptor, c-Jun, c-Fos, Jun kinase, GRB2, SOS-I, ERK-I, ERK, JAK2, STAT4, IL-12, IL-12 receptor, nitric oxide synthase, TYK2, IFNγ, elastase, IL-8, epithelins, IL-2, IL-2 receptor, CD28, SMAD3, SMAD4, TGFβ or TGFβ receptor; any cell cycle regulation, signaling or differentiation pathway including but not limited to those involving TNFs, SRC protein kinase, Cdc2, cyclin B, Grb2, Sos-1, SHC, p68, Aurora kinases, protein kinase A, protein kinase C, Eg7, p53, cyclins, cyclin-dependent kinases, neural growth factor, epidermal growth factor, retinoblastoma protein, ATF-2, ATM, ATR, AKT, CHKI, CHK2, 14-3-3, WEEI, CDC25 CDC6, Origin Recognition Complex proteins, pI5, pI6, p27, p21, ABL, c-ABL, SMADs, ubiquitin, SUMO, heat shock proteins, Wnt, GSK-3, angiotensin, p73 any PPAR, TGFα, TGFβ, p300, MDM2, GADD45, Notch, cdc34, BRCA-I, BRCA-2, SKPI, the proteasome, CULI, E2F, pi 07, steroid hormones, steroid hormone receptors, IκBα, IκBβ, Sin3A, heat shock proteins, Ras, Rho, ERKs, IKKs, PI3 kinase, Bcl-2, Bax, PCNA, MAP kinases, dynein, RhoA, PKAc, cyclin AMP, FAK, PIP2, PIP3, integrins, thrombopoietin, Fas, Fas ligand, PLK3, MEKs, JAKs, STATs, acetylcholine, paxillin calcineurin, p38, importins, exportins, Ran, Rad50, Rad51, DNA polymerase, RNA polymerase, Ran-GAP, Ran-GEF, NuMA, Tpx2, RCCI, Sonic Hedgehog, Crml, Patched (Ptc-1), MPF, CaM kinases, tubulin, actin, kinetochore-associated proteins, centromere-binding proteins, telomerase, TERT, PP2A, c-MYC, insulin, T cell receptors, B cell receptors, CBP, 1KB, NFKB, RACI, RAFI, EPO, diacylglycerol, c-Jun, c-Fos, Jun kinase, hypoxia-inducible factors, GATA4, β-catenin, α-catenin, calcium, arrestin, survivin, caspases, procaspases, CREB, CREM, cadherins, PECAMs, corticosteroids, colony-stimulating factors, calpains, adenylyl cyclase, growth factors, nitric oxide, transmembrane receptors, retinoids, G-proteins, ion channels, transcriptional activators, transcriptional coactivators, transcriptional repressors, interleukins, vitamins, interferons, transcriptional corepressors, the nuclear pore, nitrogen, toxins, proteolysis, or phosphorylation; or any metabolic pathway including but not limited to those involving the biosynthesis of amino acids, oxidation of fatty acids, biosynthesis of neurotransmitters and other cell signaling molecules, biosynthesis of polyamines, biosynthesis of lipids and sphingolipids, catabolism of amino acids and nutrients, nucleotide synthesis, eicosanoids, electron transport reactions, ER-associated degradation, glycolysis, fibrinolysis, formation of ketone bodies, formation of phagosomes, cholesterol metabolism, regulation of food intake, energy homeostasis, prothrombin activation, synthesis of lactose and other sugars, multi-drug resistance, biosynthesis of phosphatidylcholine, the proteasome, amyloid precursor protein, Rab GTPases, starch synthesis, glycosylation, synthesis of phosphoglycerides, vitamins, the citric acid cycle, IGF-I receptor, the urea cycle, vesicular transport, or salvage pathways. It is further contemplated that nucleic acids molecules of the invention can be employed in diagnostic and therapeutic methods with respect to any of the above pathways or factors. Thus, in some embodiments of the invention, a miRNA inhibits, eliminate, activates, induces, increases, or otherwise modulates one or more of the above pathways or factors is contemplated as part of methods of the invention. The nucleic acid can be used to diagnosis a disease or condition based on the relation of that miRNA to any of the pathways described above.

### FIGURE LEGENDS

FIG. 1. HPRT1 mRNA expression in the subq human A2058 melanoma tumors at 47-49 h after the last injection of 3 daily consecutive injections of siHPRT1 at 3 mg/kg.

FIG. 2. Relative tumor volume 12 days after the start of the treatment. Mice bearing subq human A2058 melanoma tumors were treated with 3 mg/kg of miRNA-193a formulated in diamino lipid nanoparticles for 5 consecutive days in week 1, followed by twice weekly injections (Monday/Thursday). Data represent medians+IQR (n=8).

FIG. 3. AFP levels (Day 42, left) and tumor weights (Day 49, right) of orthotopic Hep3b tumor bearing mice treated with different doses of miR-7 and miR-193a formulated in Nov340 or diamino lipid nanoparticles for 3 or 5 consecutive days in week 1, followed by twice weekly injections (Monday/Thursday) for another 3 weeks, as compared to PBS or Sorafenib treated mice. Data represent medians+IQR (n=6-16). *=p<0.05, **=p<0.01, ***=p<0.001, ****=p<0.0001

FIG. 4. Ratio of CD8+ T cells/Treg cells 1 (A) and 2 (B) weeks post treatment start. miRNA-193a treatment resulted in a shift from an immunosuppressive to an immunostimulatory 4T1 tumor microenvironment (CD8+ T cells/Treg cell >1, 2 weeks post start of miRNA-193a treatment).

FIG. 5. Percentage of immune cells and intracellular cytokines in CD45+ tumor cell population. Week 1: A) miRNA-193a treatment resulted in a significant increase in T-cell function (production of IFNγ and IL-2), B) and a significant decrease in regulatory T cell population (FOXP3+/LAG3+).

FIG. 5C. Week 2: miRNA-193a treatment resulted in a significant increase in T-cell frequency (CD8+) along with a mild induction in T-cell function (IFNγ).

FIG. 5D. Week 2: miRNA-193a treatment resulted in a significant decrease in regulatory T cell population (FOXP3+/LAG3+). Data represent medians+IQR. *=p<0.05, **=p<0.01.

FIG. 6. Percentage of CD73 (NTSE) expression level in immune cells. Upon miRNA-193a treatment CD73 expression level is down-regulated in immune cells. Data represent medians+IQR. *=p<0.05, **=p<0.01. A) week 1, 48h after 2^{nd} administration; B) week 2, 48h after 4^{th} administration.

FIG. 7. Percentage of mice that show primary tumor regrowth post 4T1 tumor resection. Mice were injected with 4T1 cells in mammary fat pad, twice weekly treatment (i.v.) started 1 week post cell injection, primary tumor removal at day 20 post cell injection. After primary tumor removal mice were treated for a further 6 weeks at twice weekly schedule at 10 mg/kg of miR-193a formulated in diamino lipid nanoparticles, as compared to PBS or Anti-PD1 treated mice, or treated with a combination.

FIG. 8. Individual mice with primary tumor regrowth post 4T1 tumor resection. Mice were injected with 4T1 cells in mammary fat pad, twice weekly treatment (i.v.) started 1 week post cell injection, primary tumor removal at day 20 post cell injection. After primary tumor removal mice were treated for a further 6 weeks at twice weekly schedule at 10 mg/kg of miR-193a formulated in diamino lipid nanoparticles, as compared to PBS or Anti-PD1 treated mice, or treated with a combination. 5 out of 11, 1 out of 10, 6 out of 12, and 3 out of 11 mice showed a tumor re-growth after primary tumor removal (followed up to indicated dates post-treatment) in groups 1,2,3 and 4 respectively. A) group treated with PBS; B) group treated with miRNA-193a in diamino lipid nanoparticles; C) group treated with anti-PD-1; D) group treated with a combination of miRNA-193a in diamino lipid nanoparticles and anti-PD-1.

FIG. 9. Percentage of mice that show primary tumor regrowth post 4T1 tumor resection (Day 66). Mice were injected with 4T1 cells in mammary fat pad, twice weekly treatment (i.v.) started 1 week post cell injection, primary tumor removal at day 20 post cell injection. After primary tumor removal mice were treated for a further 6 weeks at twice weekly schedule at 10 mg/kg of miR-193a formulated in diamino lipid nanoparticles, as compared to PBS or Anti-PD1 treated mice.

FIG. 10. The surviving miRNA-193a treated mice were re-challenged with 4T1 cells on day 75, 14 days post end of treatment as depicted in the left. Tumor volume is depicted in the middle and right graph as compared to naïve mice challenged with 4T1 cells. ***=p<0.001

FIG. 11. Detailed tumor volume (from FIG. 10) of the 3 miRNA-193a treated mice that showed tumor take compared to naïve mice when re-challenged with 4T1 cells.

FIG. 12. A) The surviving miRNA-193a treated mice were re-challenged with H22 cells on day 101, 38 days post end of treatment. B) Tumor volume as compared to naïve mice challenged with H22 cells. ***=p<0.001. C) Detailed tumor volume of the miRNA-193a treated mice that showed tumor take (100%) compared to naïve mice when re-challenged with H22 cells, with pronounced time-dependent tumor regression after 1 week in all miRNA-193a treated animals.

FIG. 13. Relative tumor volume 21 days after the start of the treatment. Mice bearing subq human A2058 melanoma tumors were treated with different doses and different regimen of miRNA-193a formulated in diamino lipid nanoparticles, or with vemurafenib. Data represent medians+IQR. *=p<0.05

FIG. 14. miRNA-193a target gene expression levels in the tumor over time after QDx2 (one injection per day, for two consecutive days) i.v. injection at 10 mg/kg. Mice bearing orthotopic 4T1 tumors were treated similarly and tumors were removed for pharmacodynamical analysis at different time points (see table 13). Individual tumor expression values are presented. Different target genes are significantly down-regulated at different time points. Data represent medians+IQR. *=p<0.05, **=P<0.01. Hours on axis indicate time post final miRNA-193a dose. A) mKRAS; B) mMCL-1; C) mTIM3; D) mENTPD1.

FIG. 15. miRNA-193a-3p directly targets NT5E gene and downregulates gene expression of both NT5E and ENTPD1 in different cell lines. A) luciferase activity of the NT5E wildtype reporter compared with NT5E mutated reporter in the presence of 10 nM of miRNA-193a-3p, mock and scrambled control in Hela cells. NT5E wildtype reporter reduces the luciferase activity compared with the mutated NT5E reporter and the controls. B) NT5E mRNA downregulation in the presence of 10 nM of miRNA-193a-3p compared with mock control in A2058 melanoma cell line. C) NT5E protein downregulation at 24h and 48h post administration of 10 nM miRNA-193a-3p compared with mutated miRNA-193a-3p, mock and scrambled control in A2058 melanoma cell line. Tubulin is used as loading control. Mutated miR-193a-3p contains 3 nucleotide mutations at its seed sequence D) ENTPD1 mRNA downregulation in the presence of 10 nM of miRNA-193a-3p, mock control, and scrambled control (not shown) in indicated cancer cell lines. All the mRNA values are normalized to mock (mock value set at 1).

FIG. 16. miRNA-193a-3p treatment affects adenosine generation pathway. A) free phosphate generation (indirect read out for adenosine generation) reduces upon treatment with 10 nM miRNA-193a-3p as compared with control conditions (Untreated (UT), mock, scrambled) in A2058 melanoma cells. siRNA against NTSE phenocopies the same phenotype. B) Adenosine generation reduces upon treatment with 10 nM miRNA-193a-3p compared with control conditions (Untreated (UT), mock, scrambled) in A2058 melanoma cells. siRNA against NTSE phenocopies the same phenotype. C) migration ability of A2058 cells reduces upon treatment with 10 nM miRNA-193a-3p compared with scrambled in A2058 melanoma cells. siRNA against NTSE phenocopies the same phenotype.

FIG. 17. miRNA-193a-3p enhances the G2/M arrest in cancer cells (A: HEP3B; B: SNU449; C: A2058) in a concentration-dependent manner compared to mock control, as determined by imaging of nuclei. G0, G1, S, G2/M are different phases of the cell cycle.

FIG. 18. G2/M related miRNA-193a-3p targets genes (MPP2, STMN1, YWHAZ, and CCNA2) are down-regulated upon 10 nM administration of miRNA-193a-3p in different cancer cells (A: HEP3B; B: SNU449; C: H1975) compared to mock at different time points (in hours), as determined by RT-PCR.

FIG. 19. A) surviving miRNA-193a treated mice and age matched naïve mice were re-challenged with 4T1 cells. B) miRNA-193a treated 4T1 re-challenged survivors and a group of age-matched naïve mice were depleted for T cells, re-challenged for 4T1 tumor cells and followed for tumor growth.

FIG. 19C. Mice with T-cell transfer from the miRNA-193a treated 4T1 re-challenged survivors into naïve mice, re-challenged by 4T1 and followed by tumor growth (miR-193a refers to miR-193a-3p together with formulation).

FIG. 20. AFP levels (Day 39, left) and tumor weights (Day 39, right) of orthotopic Hep3b tumor bearing mice treated with different microRNAs formulated in Nov340, every other day for the period of 3 weeks, as compared to PBS or Sorafenib treated mice. Data represent medians *=p<0.05, **=p<0.01, ***=p<0.001, ****=p<0.0001

### EXAMPLES

### Example 1—Provision of Diamino Lipids

### General Method for Providing Diamino Lipids of General Formula (I)

Alcohols of the corresponding formula T¹-OH, T²-OH, or T³-OH are generally commercially available.

These alcohols can be converted to corresponding aldehydes using methods known in the art, such as using pyridinium chlorochromate, or aldehydes may even be commercially available. The aldehydes can then be reacted with the required diamine, such as with N-,ethyl-1,3-diaminopropane when n=1 to form imines, which are subsequently reduced to corresponding amines in reductive amination of the T¹, T², and T³ moieties. The following is a non-limiting example:

### Farnesal (or Farnesyl Aldehyde)

To a mixture of farnesol (or farnesyl alcohol) (10.0 g, 44.9 mmol, 1 eq), sodium carbonate (2.38 g, 22.5 mmol, 0.5 eq) and molecular sieves 3 A (5 g) in 500 mL of dichloromethane pyridinium chlorochromate (PCC, 14.5 g, 67.4 mmol, 1.5 eq) was added. The suspension was stirred for 1h at room temperature. Then 250 mL of dichloromethane was added to the mixture and the suspension was filtrated through 250 mL of silica gel (silica gel 60, 0.04-0.063 mm, 230-400 mesh). The solvent was evaporated under reduced pressure, the residue obtained was the aldehyde and it was used without further purification. The compound was analyzed by TLC plates using a 10% ethyl acetate in cyclohexane solvent system (Rf=0.5) and 10% sulfuric acid in methanol for staining.

### (N′-methyl-N′,N″,N″-tris((2E,6E)-3,7,11-trimethyldodeca-2,6,10-trien-1-yl)propane-1,3-diamine) (Compound of General Formula (I) Wherein n=1 and Each of T¹, T², and T³ are Farnesyl)

To a solution of farnesal (8.75 g, 39.7 mmol, 3.5 eq) and N-methyl-1,3-diaminopropane (1 g, 11.3 mmol, 1.0 eq) in 100 mL 1,2-dichloroethane was added NaBH(OAc)₃ (10.9 g, 51.4 mmol, 4.55 eq) and acetic acid (2.94 mL, 51.4 mmol, 4.55 eq) at room temperature. The reaction mixture was stirred for 18 h at room temperature. The reaction was quenched with sodium hydroxide 2M solution and the mixture was extracted twice with dichloromethane (100 mL). The combined organic layers were washed with brine (saturated aqueous solution of sodium chloride) and dried over Na₂SO₄. The solvent was removed under reduced pressure, and the crude product was subject to flash chromatography on silica gel (300 mL silica gel 60, 0.04-0.063 mm, 230-400 mesh). The product was eluted employing a gradient from ethyl acetate to 4% methanol (MeOH) in ethyl acetate containing 0.5% trimethylamine in 10 column volumes. When the desired compound was eluting 20 mL fraction were collected, before the compound elutes 50-100 mL fractions were collected. Collected fractions were analyzed on TLC plates using a 5% MeOH in dichloromethane solvent system and 10% sulfuric acid in methanol for staining (Rf=0.4). The title compound was obtained as pale yellow oil (˜4.6 g, 6.6 mmol, yield 60%; chemical formula: C₄₉H₈₄N₂; exact mass: 700.66) with a typical purity of 96% as measured by RP-HPLC.

### Example 2—Provision of Nanoparticles

### General Procedures

All plastic vials and bottles were rinsed with sterile filtered deionized water prior to use. The standard error for weight in g scale is 0.01 g and for mg is 0.001 g.

### 50 mM Citrate Buffer pH 3

To 800 mL of sterile deionized water was added 10.51 g of citric acid monohydrate and 0.93 g of NaOH. The pH was measured, and if necessary it was adjust to pH 3 with 2M NaOH. Sterile deionized water was added to make 1 L. The buffer was filtrated thought a 0.2 μm bottle top filter rinsed with 20 mL sterile filtered deionized water prior to sample filtration.

### 1×PBS Buffer pH 7.4

10 g of PBS Dulbecco w/o Ca²⁺ w/o Mg²⁺ were dissolved in 10 L of sterile deionized water.

### Particle Production

Stock solutions of diamino lipid, 1,2-distearoyl-sn-glycero-3-phosphocholine (DSPC), cholesterol, and PEG2000-DSG were prepared at concentrations of 50 mM in ethanol and mixed to yield a molar ratio of 40:10:48:2 respectively. The final lipid mix solution was diluted with ethanol to a concentration of 33.8 mM. Nucleic acid (RNA) stock solution at a concentration of 20 mg/mL in H₂O was diluted in 50 mM sodium citrate buffer, pH 3 to a final concentration of 0.65 mg/mL. The total lipid to RNA mass ratio was 10.3.

To prepare the lipid nanoparticles, the organic lipid mix solution was injected into aqueous RNA solution to afford a final suspension containing 25% ethanol. The solutions were injected using a HPLC pump (Pump P-900, GE Healthcare, Germany) at relative volumetric flow of 3:1 (18.75 mL/min aqueous solution: 6.25 mL/min organic solution) and mixed via a T-junction (PEEK Low Pressure Tee Assembly 1/16″ PEEK 0.020 thru hole, IDEX Health & Science LLC, USA).

The nanoparticle suspension was immediately dialyzed 2 times against PBS buffer pH 7.4 at 200× volumes of the nanoparticle solution using a 70 mL Slide-A-Lyzer with a MWCO of 10 kD to remove ethanol and achieve buffer exchange. The first dialysis was performed at room temperature for 4 h and then the formulations were dialyzed overnight at 4° C. The resulting nanoparticle suspension was concentrated by centrifugation using VIVASPIN 20 concentrators. The concentrators were rinsed with 2 mL 1×PBS pH 7.4 prior filling the formulation (maximum of 20 mL of the formulation). The concentrators were spun at 1000 g with a swing rotor on a Heraeus Multifuge X3 FR centrifuge (Thermo Fisher Scientific, Germany) at 4° C. until the desired concentration was achieved (e.g 2 mg/mL). Aliquots at different concentration were prepared by diluting the concentrated formulation (e.g 2 mg/L) with sterile filtrated 1×PBS buffer pH 7.4. The resulting nanoparticle suspension was filtered through 0.2 μm sterile filter into glass vials and sealed with a crimp closure. Table 1 shows examples of further nanoparticles that were prepared.

TABLE 1
compositions of nanoparticles in mol %

| Entry | Diamino | Phos- | | Choles- | PEG- | Lipid/RNA |
|---|---|---|---|---|---|---|
| # | lipid | pholipid | | terol | anchor | (weight ratio) |
| 1 | 40 | 10 | (DSPC) | 48 | 2 (DSA) | 10 |
| 2 | 40 | 10 | (DSPC) | 48 | 2 (DSA) | 7 |
| 3 | 40 | 20 | (DSPC) | 38 | 2 (DSA) | 7 |
| 4 | 35 | 20 | (DSPC) | 43 | 2 (DSA) | 7 |
| 5 | 40 | 20 | (DOPE) | 38 | 2 (DSA) | 7 |
| 6 | 40 | 20 | (DSPC) | 36 | 4 (DSA) | 7 |
| 7 | 40 | 5 | (DSPC) | 48 | 2 (DSA) | 7 |
| 8 | 45 | 10 | (DSPC) | 53 | 2 (DSA) | 7 |
| 9 | 35 | 5 | (DSPC) | 53 | 2 (DSA) | 7 |
| 10 | 40 | 5 | (DSPC) | 54 | 1 (DSA) | 7 |
| 11 | 40 | 0 | | 58 | 2 (DSA) | 7 |
| 12 | 40 | 10 | (DSPC) | 48 | 2 (DPG) | 7 |
| 13 | 40 | 10 | (DSPC) | 48 | 2 (DSA) | 6 |

The size and the polydipsersity index (PDI) of the particles were measured by dynamic light scattering (DLS) technique using a Zetasizer Nano ZSP, ZEN5600, Malvern Instruments Ltd., U.K. with, He—Ne laser (633 nm). DLS measures the diffusion of particles moving under Brownian motion, and converts this to size and a size distribution using the Stokes-Einstein relationship. The measurements were performed in triplicate at a scattering angle of 173° at 25° C. and using clear disposable cuvette (10×10×48 mm, Sarstedt). The samples were 100 fold diluted with PBS buffer pH 7.4 prior to the measurement. The analysis was carried out using the Malvern software (DTS v 7.11, Malvern Instrument, UK) in multiple narrow mode analysis. The results are the average of the triplicate measurements and expressed as z-average diameter and PDI.

Zeta potential of nanoparticles was measured by the same Zetasizer using M3-PALS technique. The zeta potential of particles is calculated by determining the electrophoretic mobility of the particles and applying Henry's equation. Electrophoretic mobility is obtained by measuring the velocity of the particles while they are moving due to electrophoresis. The electrophoretic mobility was determined in an aqueous medium, using Smoluchowski approximation. The measurements were performed in triplicate at 25° C. in a clear disposable folded capillary cell (DTS1070, Malvern Instrument, UK). The samples were 100 fold diluted with 0.1×PBS buffer pH 7.4 prior to the measurement. The analysis was carried out using the Malvern software (DTS v 7.11, Malvern Instrument, UK) in auto mode analysis. The results are the average of the triplicate measurements and expressed as zeta potential.

Nucleic acid concentration (measuring total RNA) was determinate by UV-Vis spectrophotometry using a DU 800 spectrophotometer (Beckman Coulter, Beckman Coulter, Inc., Brea, Calif.). The absorbance of a diluted RNA sample is measured at 260 nm and the concentration is calculated using the Beer-Lambert law. Briefly, 100 μL of the diluted formulation in 1×PBS was added to 900 μL of a 4:1 (v/v) mixture of methanol and chloroform to dissolve the LNP. After mixing, the absorbance spectrum of the solution was recorded between 230 nm and 330 nm using a quartz cuvette (10 mm length path, 12.5×12.5×45 mm, Helima). The RNA concentration in the formulation was calculated based on the extinction coefficient of the RNA used in the formulation and on the difference between the absorbance at a wavelength of 260 and the baseline corrected value at a wavelength of 330 nm. The extinction coefficient of the RNA is determinate by measuring the absorbance at 260 nm of 6 RNA solutions at different concentrations ranging from 0.005 to 0.05 mg/mL and applying the Beer-Lambert law.

RNA encapsulation efficiency was evaluated by the Quant-iT™ RiboGreen® RNA assay. Briefly, the samples were diluted to a concentration of approximately 5 ng/mL in Tris-EDTA (TE) buffer pH 7.5. 50 μL of the diluted samples were transferred to a polystyrene 96 well plate, then either 50 μL of TE buffer (measuring unencapsulated RNA) or 50 μL of a 2% Triton X-100 solution (measuring total RNA, both encapsulated within LNPs and un-encapsulated, “free” RNA) was added. Samples were prepared in triplicate. The plate was incubated at a temperature of 37° C. for 15 minutes. The RiboGreen reagent was diluted 1:100 in TE buffer, 100 μL of this solution was added to each well. The fluorescence intensity was measured using a fluorescence plate reader (Wallac Victor 1420 Multilablel Counter; Perkin Elmer, Waltham, Mass.) at an excitation wavelength of ˜480 nm and an emission wavelength of ˜520 nm. The fluorescence values of the reagent blank were subtracted from that of each of the samples and the encapsulation efficiency was determined as follows:

Encapsulation efficiency=(1−([unencapsulated RNA]/[total RNA]))*100

Table 2 shows analytic values for the resulting nanoparticles, including their polydispersity (PDI).

TABLE 2
properties of nanoparticles; entry numbers
correspond to those in table 1

| | | | Zeta potential | Encapsulation |
|---|---|---|---|---|
| Entry | Mean size | | at pH 7.4 | efficiency |
| # | (nm) | PDI | (mV) | (RiboGreen %) |
| 1 | 70 | 0.06 | −0.5 | 98 |
| 2 | 58 | 0.05 | 0.2 | 78 |
| 3 | 68 | 0.07 | −0.7 | 97 |
| 4 | 69 | 0.06 | −1.9 | 80 |
| 5 | 80 | 0.02 | −1.4 | 99 |
| 6 | 52 | 0.07 | −3.4 | 94 |
| 7 | 77 | 0.03 | 0.0 | 99 |
| 8 | 55 | 0.03 | 0.5 | 94 |
| 9 | 68 | 0.03 | −0.3 | 100 |
| 10 | 86 | 0.03 | −0.3 | 100 |
| 11 | 85 | 0.02 | 0.0 | 99 |
| 12 | 62 | 0.04 | −0.2 | 96 |
| 13 | 59 | 0.04 | −2.1 | 99 |

### Reference Nanoparticles

As a reference, so-called Nov340 lipid nanoparticles were also prepared. The composition of Nov340 lipid nanoparticles has been described in U.S. Pat. No. 9,737,482 comprising of the following types of lipids: an amphoteric lipid pair (a cationic and an anionic lipid) and neutral lipids. The lipid composition of Nov340 lipid-nano particle is as follows:
POPC Palmitoyl-oleoylphosphatidylcholine
DOPE Dioleoylphosphatidylethanolamine
CHEMS Cholesterolhemisuccinate
MoChol 4-(2-Aminoethyl)-Morpholino-Cholesterolhemisuccinate

The Nov340 lipid mixture consists in mol. % of the following ratio: 6 (POPC), 24 (DOPE), 23 (CHEMS), and 47 (Mochol). The formulation of the Nov340 lipid nanoparticles is based on the method described in U.S. Pat. No. 6,843,942.

Lipids (POPC, Chems, and DOPE) are dissolved in absolute EtOH in a heating cabinet at 55° C. After complete dissolution of the lipids, this solution is transferred quantitatively into another bottle where MoChol is already weighed. This lipid mixture is stirred at 55° C. until MoChol dissolves. MOChol and Chems were obtained from Merck and POPC and POPE from Avanti Polar Lipids. Dissolution of lipids in two steps is done to decrease the degradation of MoChol to Chol. The final lipid solution is then filtered through a 0.2 μm pore size filter into the preparation system which is pre-heated at 55° C. In parallel, the oligonucleotide is dissolved in Na-Acetate/Sucrose pH 4 buffer at room temperature (RT) and filtered through a 0.2 μm pore size filter directly into the API bottle.

Liposomes form at the site of injection when lipid solution and API solution are combined using the method as described in U.S. Pat. No. 6,843,942. Immediately after liposome formation the suspension is diluted with NaCl/Na2HPO4 pH 9.0 buffer to increase the pH of the formulation to pH 7.5. The injection buffer as well as the dilution buffer are kept at RT. The generated liposomes (intermediate volume) are collected in a bottle. Liposomes are stirred for 30 minutes at RT before extrusion.

The intermediate volume is extruded through 200 nm pore size polycarbonate membranes to refine its size and size distribution. Both parameters are important to allow a final 0.2 μm sterile filtration and to decrease product losses.

Ultra-/diafiltration using a hollow fiber membrane (100 kDa MWCO; Merck Millipore) was performed to remove the free RNA and EtOH from the liposomal sample. During ultrafiltration the sample was concentrated down to the target volume (to achieve the target RNA concentration) and then during diafiltration, 10 volume exchanges were performed with PB Sucrose pH 7.5 to ensure complete removal of EtOH and free oligonucleotides and to exchange the outer buffer. Liposomes were 0.2 μm filtered using a syringe filter and filled into sterile vials. The vials remained sealed and stored at 2-8° C. protected from light.

Size measurement: Measurement for size/Pd I determination of liposomes was performed by Dynamic-Laser-Light-Scattering (DLS) using a Zetasizer Nano ZS (Malvern). This system is equipped with a 4 mW Helium/Neon Laser at 633 nm wavelength and measures the liposome samples with the non-invasive backscatter technology at a detection angle of 173°. Liposomes were diluted in purified water to reach optimal liposome concentration and the experiments were carried out at 25° C.

Zeta potential: Zeta potential of liposomes was measured using a Zetasizer Nano ZS (Malvern)

Quantification of RNA: Quantification of RNA was done by spectrophotometry at OD: 260 nm. The formulated lipid-nano particles were first diluted with purified water and then with methanol/chloroform to lyse the liposomes and release the amount of encapsulated RNA.

Quantification of lipid: Lipid concentration in the samples was measured from the bulk volume using HPLC method.

### Example 3—Provision of Oligonucleotides

For all miRNA and siRNA molecules used in this invention, such as for example miR-193a-3p, miR-7-5p, and HPRT1 siRNA, passenger and guide strands are chemically synthesized by solid phase synthesis using a commercially available synthesizer such as the Oligopilot 400 oligonucleotide synthesizer. The processes used to manufacture the single strands, are commonly used in industry to produce si-/miRNA oligonucleotides. Following the synthesis, the oligonucleotide single strands are cleaved from the solid support and are deprotected. The crude single oligonucleotide strands are purified using HPLC. Thereafter the single strands are desalted, concentrated, annealed, and lyophilized. Throughout these examples, unless context makes it clear that something else is intended, miR-193a refers to a duplex of the miRNA-193a-3p of SEQ ID NO: 218 (mimic, sense) with an antisense strand of SEQ ID NO: 219. It is used naked in in vitro studies, or together with the formulation in in vivo studies.

### Example 4—Pre-Clinical In Vivo Mouse Experiments

### Material and Methods

RNA Isolation

Total RNA was isolated from tumors using TriZol (Thermo Fisher) according to the manufacturer's instructions. Isolated RNA was resuspended in nuclease-free water (NFW).

RT-qPCR

To prepare cDNA, first 100 ng total RNA was mixed with Random Hexamers (Qiagen; final concentration 2 μM) in a final volume of 12.5 μl in NFW, denatured for 5 min at 70° C. and immediately cooled on ice. Next, 7.5 μl of a cDNA synthesis mix was added, consisting of 4 μl 5×RT Buffer (Promega), 0.4 μl 25 mM dNTPs (Promega), 1 μl 200 U/μL MMLV RT-enzyme (Promega), 0.5 μL 40 U/μL RNAse Inhibitor (Promega) and 1.6 μL NFW. The following cDNA synthesis protocol was used:

| 1. 10 minutes | 25° C. |
|---|---|
| 2. 60 minutes | 37° C. |
| 3. 5 minutes | 85° C. |
| 4. ∞ | 4° C. |

For a single qPCR reaction the following mix was prepared:

| | | |
|---|---|---|
| 1. cDNA | 1 | μL |
| 2. Forward primer (250 μM) | 0.05 | μL |
| 3. Reverse primer (250 μM) | 0.05 | μL |
| 4. NFW | 8.9 | μL |
| 5. SYBR Green (Bio-Rad) | 10 | μ L |

The following qPCR protocol was used:

1. One cycle: 5 minutes 95° C.
2. 40 cycles: 15s 95° C.+30s 60° C.

Each sample was analyzed as technical triplicate on a CFX96 Real-Time qPCR machine (Bio-Rad). HPRT1 expression was calculated using 2-(CtHPRT1—GEOMEAN(CtUBC;CtGUSB)). Primers used in qPCR are shown below:

| Gene | Primer | Sequence (5′-3′) | SEQ ID NO: |
|---|---|---|---|
| HPRT1 | Forward | TCCAAAGATGGTCAAGGTCGC | 222 |
| | Reverse | AGTCAAGGGCATATCCTACAACAA | 223 |
| UBC | Forward | CAGCCGGGATTTGGGTCG | 224 |
| | Reverse | CACGAAGATCTGCATTGTCAAGT | 225 |
| GUSB | Forward | TGCGTAGGGACAAGAACCAC | 226 |
| | Reverse | GGGAGGGGTCCAAGGATTTG | 227 |
| mPPIH | Forward | AATCGAGCTCTTTGCAGACG | 228 |
| | Reverse | TATCCTATCGGAACGCCATC | 229 |
| mSDHA | Forward | GAGGAAGCACACCCTCTCAT | 230 |
| | Reverse | GGAGCGGATAGCAGGAGGTA | 231 |
| mMCL-1 | Forward | TAAGGACGAAACGGGACTGG | 232 |
| | Reverse | CGCCTTCTAGGTCCTGTACG | 233 |
| mENTPD1 | Forward | GCCGAATGCATGGAACTGTC | 234 |
| | Reverse | CTGCCGATTGTTCGCTTTCC | 235 |
| mKRAS | Forward | GTGGATGAGTATGACCCTACGA | 236 |
| | Reverse | CTCCTCTTGACCTGCTGTGT | 237 |
| mTIM3 | Forward | GCAGGATACAGTTCCCTGGT | 238 |
| | Reverse | TCTGAGCTGGAGTGACCTTG | 239 |
| hMpp2 | Forward | CCAGGATGATGCCAACTGGT | 240 |
| | Reverse | ATGCTTTCCGCTTCTCCTCC | 241 |
| hSTMN1 | Forward | CCAGAATTCCCCCTTTCCCC | 242 |
| | Reverse | CCAGCTGCTTCAAGACCTCA | 243 |
| hYWHAZ | Forward | AGAAAATTGAGACGGAGCTAAGAGA | 244 |
| | Reverse | AGAAGACTTTGCTCTCTGCTTGTG | 245 |
| hCCNA2 | Forward | CGGTACTGAAGTCCGGGAAC | 246 |
| | Reverse | TGCTTTCCAAGGAGGAACGG | 247 |
| hNT5E | Forward | AACAACCTGAGACACACGGA | 248 |
| | Reverse | TGGATTCCATTGTTGCGTTCA | 249 |
| hENTPD1 | Forward | GCTTCTTGTGCTATGGGAAGGA | 250 |
| | Reverse | GATGAAAGCATGGGTCCCTGA | 251 |

Determination of Tumor Growth Inhibition (TGI) Effects:

To determine TGI effects, the T/C (tumor/control) ratio was determined by calculating the relative percentage increase in TV (tumor volume) for the individual mice of each group (TV of the day of randomization as reference point), and then dividing the group median relative increase in TV for the treated groups by that for the PBS group. Outliers (with respect to individual TVs) within each treatment group were determined by using the formulas Q1−1.5×IQR (lower limit) and Q3+1.5×IQR (upper limit).

Statistical Analysis:

Statistical analysis was performed using Graphpad Prism 7. A two-tailed, non-parametric Mann-Whitney test was used to calculate differences in relative tumor volumes (TV on day ‘X’ divided by TV on the day of randomization). In all statistical tests, the effect of the test items was compared to the PBS group. A p-value <0.05 was considered statistically significant.

For all miRNA and siRNA molecules used in this invention, such as for example miR-193a-3p, miR-7-5p, and HPRT1 siRNA, passenger and guide strands are chemically synthesized by solid phase synthesis using a commercially available synthesizer such as the Oligopilot 400 oligonucleotide synthesizer. The processes used to manufacture the single strands, are commonly used in industry to produce si-/miRNA oligonucleotides. Following the synthesis, the oligonucleotide single strands are cleaved from the solid support and are deprotected. The crude single oligonucleotide strands are purified using HPLC. Thereafter the single strands are desalted, concentrated, annealed, and lyophilized.

FACS Analysis on Tumor Samples

Freshly isolated orthotopic 4T1 tumors were prepared for FACS analysis on day 5 (week 1) and day 12 (week 2) post miRNA-193a treatment when they reached a min tumor volume (TV) of 300 mm³. Tumor samples were digested using murine tumor dissociation kit from Miltenyi (CAT #130-096-730). Following tumor cell digestion cells were re-suspended in 200 μl staining buffer with 1 pg/ml Fc-Block (Mouse BD Fc Block™ CAT #553141) and incubated at 4° C. for 15 minutes in the dark. Different markers (CD45, CD3, CD4, CD8, FoxP3, CD335, F4/80, CD11b, Gr-1, CD73, LAG3/CD223, IL-2, IFN-r, PD-1, L/D stain) have been used for FACS analysis. Antibody mixture for all markers except IL-2, IFN-r and LAG3 were diluted in Fc blocking buffer for each sample and stained for 30 min on ice, in the dark. Then, cells were gently washed by adding 2 ml of ice cold PBS to each tube. Each tube was centrifuged at 300 g for 5 minutes and supernatant was discarded. To detect intracellular markers (IL-2, IFN-r and LAG3) stimulation of the digested cells was performed. To do so, Leukocyte Activation Cocktail, with BD GolgiPlug™ was used. The cocktail was thawed at 37° C. in a water bath rapidly and for every 1 mL of cell culture (e.g., ˜10⁶ cells/mL) 2 μL of cocktail was added and mixed thoroughly. To stimulate, cell culture mix was placed in a 37° C. humidified CO₂ incubator for 4-6 hr. Then the cells were harvested and washed with FACS staining buffer. For staining, cell pellet was resuspended with pulse vortex and 200 ul of prepared Fixation/Permeabilization was added to each sample. Samples were incubated for 10 min at RT in the dark. Then, samples were washed twice by adding 1 ml of 1× Permeabilization Buffer (made from 10× Permeabilization Buffer, diluted with distilled H₂O) followed by centrifugation and decanting of supernatant and each sample was incubated with the antibody cocktail containing FoxP3, IFN-r and IL-2 in 1× Permeabilization Buffer and incubated at RT for 30 minutes in the dark. Finally, cells were resuspended in 150 p1 of staining Buffer and analyzed on cytometer. The data was analysed by Kaluza flow cytometry software (from Beckman Coulter).

T-Cell Depletion and FACS Analysis on Whole Blood

In all cases, monoclonal antibodies were delivered to mice by intraperitoneal injection in 200p1 phosphate-buffered saline. For depletion and neutralization experiments, CD4 (clone GK1.5) and CD8 (clone 2.43) antibodies were used simultaneously. Depletion or neutralization was commenced a week before tumor cell inoculation. For CD4+ and CD8+ T-cell depletion, 250 pg of the indicated antibody was delivered on a QOD×3 schedule in the first week and then a Q3D schedule for 3 more weeks. Depletion of the desired T-cell population(s) was confirmed on whole blood by flow cytometry (data not shown). 100 μL whole blood was drawn per animal on day 0 and day 14 post tumor cell inoculation (day 0=24h post QOD×3, Day 14: 24h post QOD×3+Q3D for 2 weeks). Then mixed with the antibodies. After vortexing gently, the mix was incubated for 30 minutes in the dark at room temperature (RT). 2 ml of 1× FACS lysing buffer was added to the solution and incubated for 10 min at RT. Then the solution mix was centrifuged for 5 min and the supernatant was removed. Finally, the cells were resuspended in 150p1 of staining buffer and analyzed on a cytometer. All flow cytometry antibodies (anti-CD45-PerCP-cy55 (clone: 30-F11), anti-CD3-FITC (clone: 17A2) anti-CD4-APC (clone: RM4-4), anti-CD8-PE (clone: 53-6.7) were purchased from Biolegend except anti-CD3 from BD Biosciences. (QOD×3: every other day for 3 days, Q3D: every 3 days (Monday-Thursday)).

Adoptive T-Cell Transfer

Adoptive transfer of CD3+ T cells form the survivor mice into naive mice was performed after spleens, auxiliary, brachial, and inguinal lymph nodes excision. Cell suspension were made from the indicated organs and pooled. Then, CD3+ T-cells were isolated from the pool using magnetic beads according the manufacturer's protocol (Miltenyi Biotech). 1×107 CD3+ T-cells per mouse were injected intravenously.

MTS Assay

Depending on cancer cell type 3000-6000 cells were seeded in a 96 well plate. After cells attached, they were transfected with different concentrations of miRNA-193a and viability was measured at different time points. Viability measurement was performed using CellTiter 96 AQueous One Solution Cell Proliferation Assay (Promega). 20 μL of MTS reagent was added to 100 μL of media in each well in 96-well plate and incubated for 2 hours at 37° C. Absorbance was measured at 490 nM for each sample in 96 well plate reader.

Caspase GLO Caspase 3/7 Assay (Apoptosis Assay)

Depending on cancer cell type 3000-6000 cells were seeded in a 96 well plate. After cells attached, they were transfected with different concentrations of miRNA-193a and apoptosis induction was measured at different time points. This assay was performed using Promega kit. 100 uL of caspase reagent was added to 100 uL of media in each well in 96-well plate and incubated for 2 hours in the dark at room temperature. The luminescent signal was read on a luminescent plate reader.

Boyden Chamber Assay

To check the migratory ability of cells, a Boyden Chamber assay was performed, which is based on a chamber of two medium-filled compartments separated by a 0.8 μm pore size membrane (BD falcon). In this assay, depending on cell type 60,000-120,000 cells were seeded in the upper compartment of the membrane in serum free medium and were allowed to migrate through the pores of the membrane into the lower compartment, in which serum is present in the media. The serum acts as a chemoattractant. After an appropriate incubation time, the membrane between two compartments was fixed, stained and 6 different images from each membrane were taken. The number of migratory cells were counted using Image J analysis.

Nuclei Imaging (Measuring the Cell Cycle Profile)

Depending on cancer cell type 3000-6000 cells were seeded in a 96 well plate. After cells attached, they were transfected with different concentrations of miRNA-193a and cell cycle profile was measured at different time points. At selected time points media was aspirated from wells and DNA staining solution (Hoescht 33342/Saponin/PFA) was added to each well and incubated for 2 hours at 37° C. Then, cell images were taken using Thermo Celllnsite in which nuclei were specified and nuclei intensity were measured. Nuclei intensity was analyzed using program R to determine the different phases of the cell cycle (G0/G1, S, G2/M) based on the DNA content.

Annexin V/Propidium Iodide Apoptosis Assay (Flow Cytometry)

To specifically detect the cells that are undergoing apoptosis, FITC Annexin V apoptosis detection kit (BD pharmingen) was used. Depending on cancer cell type different number of cells were seeded in 6-well plate to have a confluency of about 70% at the time of measurement. After cells attached, they were transfected with different concentrations of miRNA-193a and apoptosis assay was measured at different time points following the manufacturer's protocol. With this assay we have detected the percentage of apoptotic cells indicating the SubG1 phase of the cell cycle.

3′UTR Luciferase Assay

Firefly luciferase reporter constructs containing the 3′ untranslated regions (UTR) of NT5E (CD73) were transfected in Hela cells along with 10 nM miRNA-193a-3p or a scrambled control. Cell extracts were prepared 24 hours after transfection, and luciferase activity was measured using the Dual Luciferase Reporter Assay System (Promega). If the 3′UTR is a target of the miRNA, miRNA will interact with 3′UTR and provides lower luciferase signal.

Western Blot

Approximately 1×10⁶ cells were harvested and lysed in immunoprecipitation RIPA lysis buffer and the lysates were blotted onto a PVDF membrane. Membranes were probed overnight with primary antibodies and bound antibodies were visualized using HRP-linked secondary antibodies (Cell-Signalling). The antibodies used for western blot included anti-NT5E (D7F9A) from Cell Signalling Technology, and anti-αTubulin from Santa Cruz. αTubulin was used as a loading control for the western blot experiment.

Malachite Green Phosphate Assay

2000 A2058 melanoma cells were seeded per well in a 96 well plate. 24h after seeding, cells were transfected with different concentrations of miRNA-193a-3p, scrambled control, siNT5E and siPool as control. Malachite Green Phosphate Assay (Sigma Aldrich) was used to measure the liberation of phosphate from nucleoside triphosphates ATP following inhibition of CD39 (ENTPD1) and CD73 (NT5E) as target genes of miRNA-193a-3p 24h and 48h post transfection. The rapid colour formation from the reaction can be conveniently measured on a plate reader (600-660 nm).

Adenosine Assay

4000 A2058 melanoma cells were seeded per well in a 96 well plate. 4h after seeding, cells were transfected with different concentrations of miRNA-193a-3p, scrambled control, siNT5E and siPool as control. 24h post transfection the cells were treated with 500 μM AMP and adenosine measurement was performed 24h post-treatment following steps from adenosine measurement kit (BioVision).

Cell Preparation for RNA Sequencing

Six human cancer cell lines were cultured in appropriate media (Table 3) and seeded into 6-well plates 24h before transfection with 10 nM miRNA-193a-3p or mock using Lipofectamine RNAiMAX (Thermofisher). Reagents were aspirated 16h after transfection and cells were passaged into new 6-well plates. Media was aspirated 24h after transfection and plates were stored at −80° C. Three independent replicates were performed for each cell line.

TABLE 3
Cell line details.

| Cell line | Cancer type | Medium |
|---|---|---|
| A549 | Lung (NSCLC) | F-12K + 10% FBS + P/S |
| H460 | Lung (NSCLC) | RPMI-1640 + 10% FBS + P/S |
| HEP3B | Liver (HCC) | EMEM + 10% FBS + P/S |
| HUH7 | Liver (HCC) | DMEM low glucose + 10% FBS + |
| | | P/S + L-glutamine |
| A2058 | Melanoma | DMEM + 10% FBS + P/S |
| BT549 | Breast (TNBC) | RPMI-1640 + 10% FBS + P/S + |
| | | 0.023 IU/mL insulin |

FBS: fetal bovine serum, P/S: penicillin streptomycin

RNA Isolation for RNA Sequencing

RNA was isolated using the miRNeasy Mini kit (Qiagen). The procedure included on-column DNase treatment. RNA concentration was measured on NanodropOne. 150 ng of each independent replicate was pooled and 450 ng samples (Table) were submitted to GenomeScan BV (Leiden, The Netherlands).

TABLE 4
RNA samples for RNA-sequencing

| GenomeScan ID | Customer ID |
|---|---|
| 103485-001-001 | A2058 Mock_24 |
| 103485-001-002 | A2058 miRNA-193a-3p_24 |
| 103485-001-005 | A549 Mock_24 |
| 103485-001-006 | A549 miRNA-193a-3p_24 |
| 103485-001-009 | BT549 Mock_24 |
| 103485-001-010 | BT549 miRNA-193a-3p_24 |
| 103485-001-013 | H460 Mock_24 |
| 103485-001-014 | H460 miRNA-193a-3p_24 |
| 103485-001-017 | HEP3B Mock_24 |
| 103485-001-018 | HEP3B miRNA-193a-3p_24 |
| 103485-001-021 | HUH7 Mock_24 |
| 103485-001-022 | HUH7 miRNA-193a-3p_24 |

RNA-Sequencing Procedure

PolyA enrichment was performed followed by next generation RNA-Sequencing using Illumina NovaSeq 6000 at GenomeScan BV. The data processing workflow included raw data quality control, adapter trimming, and alignment of short reads. The reference GRCh37.75.dna.primary_assembly was used for alignment of the reads for each sample. Based on the mapped locations in the alignment file the frequency of how often a read was mapped on a transcript was determined (feature counting). The counts were saved to count files, which serve as input for downstream RNA-Seq differential expression analysis.

Data Analysis for RNA Sequencing

Differential expression analysis was performed on the short read data set by GenomeScan BV. The read counts were loaded into the DESeq package v1.30.0, a statistical package within the R platform v3.4.4. DESeq was specifically developed to find differentially expressed genes between two conditions (mock versus miRNA-193a-3p) for RNA-Seq data with small sample size and over-dispersion. The differential expression comparison grouping is provided in Table.

TABLE 5
Expression comparison setup

| Comparison | Condition A | Condition B |
|---|---|---|
| 1 | A2058_Mock_24 | A2058_miRNA-193a-3p_24 |
| 2 | A549_Mock_24 | A549_miRNA-193a-3p_24 |
| 3 | BT549_Mock_24 | BT549_miRNA-193a-3p_24 |
| 4 | H460_Mock_24 | H460_miRNA-193a-3p_24 |
| 5 | HEP3B_Mock_24 | HEP3B_miRNA-193a-3p_24 |
| 6 | HUH7_Mock_24 | HUH7_miRNA-193a-3p_24 |

### Example 4.1: Comparison of LNP Efficacy in Mice Bearing Subq Human A2058 Melanoma Tumors

Four to six weeks old female athymic nude mice (Crl:NU(NCr)-Foxn1nu; Charles River) were unilaterally and subcutaneously injected in the flank with 1×107 A2058 cells in 50% matrigel (0.2 mL/mouse). At the time of randomization, the TVs ranged between 134.5-538.7 mm3 (median 213.4, IQR 178.3-265.9). Body weights and TVs (caliper measurements) were determined three times/week.

After randomization, mice received a total of three i.v. injections, each administered on three consecutive days (QDx3). For the dosing scheme see Table 6. QDx3=one injection per day for 3 consecutive days.

TABLE 6
dosing scheme; administration was iv for each group ID

| | | | | dosing | | |
|---|---|---|---|---|---|---|
| group | | | dose | volume | Schedule of | no. of |
| ID | Treatment | Vehicle | [mg/kg/d] | [ml/kg] | administration* | mice |
| 1 | PB-S | PB-sucrose | — | 5 | QDx3 | 4 |
| 2 | siHPRT1 | NOV340 | 3 | 5 | QDx3 | 4 |
| | | reference particles | | | | |
| 3 | siHPRT1 | Particles according | 3 | 5 | QDx3 | 4 |
| | | to the invention | | | | |
| | | (table 1, entry 1) | | | | |

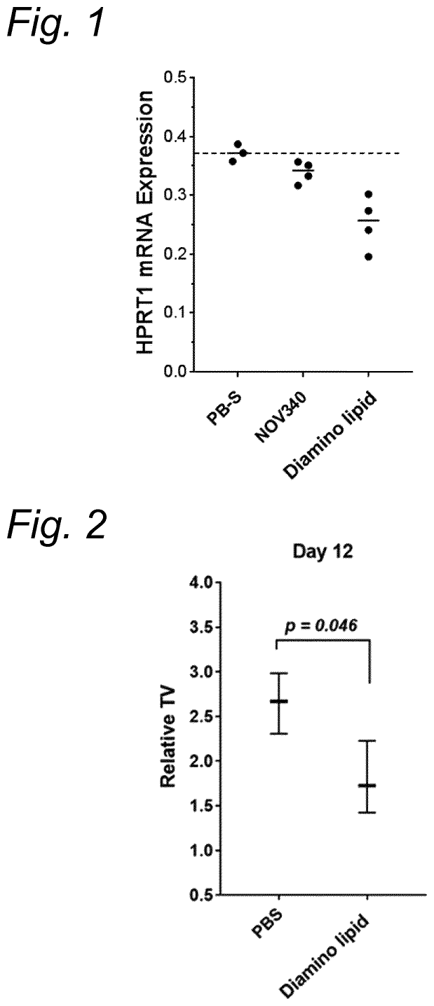

Tumors were collected between 47 and 49 h post last dose. Mice were first anaesthetized using isoflurane, and then sacrificed by cervical dislocation. Tumors were snap frozen in liquid nitrogen and stored at −80° C. FIG. 1 shows HPRT1 mRNA expression in the subq human A2058 melanoma tumors at 47-49 h after the last injection of 3 daily consecutive injections of siHPRT1 at 3 mg/kg.

### Conclusion:

Nanoparticles according to the invention mediated functional delivery of siHPRT1 to the subq tumors, while NOV340 did not (FIG. 1).

### Example 4.2: Tumor Growth Inhibitory Effects of miRNA-193a Formulated in Diamino Lipid Nanoparticles in Mice Bearing Subq Human A2058 Melanoma Tumors

Four to six weeks old female athymic nude mice (Crl:NU(NCr)-Foxn1nu; Charles River) were unilaterally and subcutaneously injected in the flank with 1×107 A2058 cells in 50% matrigel (0.2 mL/mouse). At the time of randomization, the TVs (tumor volumes) ranged between 139.4 and 245.5 mm3 (median 161.3, IQR=149.9-175.1). Body weights and TVs (caliper measurements) were determined three times/week. After randomization, the mice received a total of five daily consecutive i.v. injections in the first week, after which they received BIW maintenance dosing. For the dosing scheme see Table 7. *QDx5=one injection per day for five consecutive days; BIW=twice a week.

TABLE 7
Dosing scheme Study P508C - route was iv for each group ID

| | | | | Dosing | | |
|---|---|---|---|---|---|---|
| Group | | | Dose | volume | Schedule of | No. of |
| ID | Treatment | Vehicle | [mg/kg/d] | [ml/kg] | administration* | mice |
| 1 | PBS | PB-saline | — | 5 | QDx5, followed by BIW | 8 |
| | | | | | (Mon, Thu) x 3 weeks | |
| 2 | miR-193a-3p | Table 1 entry 1 | 3 | 5 | QDx5, followed by BIW | 8 |
| | | | | | (Mon, Thu) x 3 weeks | |

The relative tumor volumes 12 days after the start of the treatment are presented in FIG. 2. At this stage, a T/C of 0.44 was observed for the group treated with the composition according to the invention. We noticed that the BIW maintenance dosing during the remainder of the study was insufficient to support the significant TGI effects. FIG. 2 shows relative tumor volume 12 days after the start of the treatment; in this figure miR-193a refers to miR-193a-3p in lipid nanoparticle formulation. Mice bearing subq human A2058 melanoma tumors that were treated with 3 mg/kg of miRNA-193a formulated in diamino lipid nanoparticles for 5 consecutive days in week 1, followed by twice weekly injections (Monday/Thursday).

### Conclusion:

miRNA-193a formulated in nanoparticles according to the invention mediated significant TGI effects in the subq mouse model of human A2058 melanoma tumors

### Example 4.3: Tumor Growth Inhibitory Effects of miRNA-193a or miRNA-7 Formulated in Diamino Lipid Nanoparticles or in NOV340 in Mice Bearing Orthotopic Human Hep3b Hepatocellular Carcinoma Tumors

Seven to 8 weeks old female SCID/Beige mice (Shanghai Lingchang Bio-Technology Co. Ltd, Shanghai, China) were orthotopically implanted with a single 2×2×2 mm piece of a subq grown Hep3b tumor into the left liver lobe. Mice were randomized based on day 21 AFP levels. At the time of randomization, the AFP levels (ng/ml plasma) ranged between 1019 and 19779 ng/ml (median 5203, IQR=2690-9498). Treatments started on day 22 and continued for three weeks (see Table 8 for dosing scheme). AFP was measured once a week (4 times in total), and BW was measured twice a week. At the end of the study tumor weights were also determined. QDx3, QDx5=one injection per day for three or five consecutive days; BIW=twice a week; BID=twice daily; po=per oral.

TABLE 8
Dosing scheme for example 4.3

| | | | | Dosing | | | |
|---|---|---|---|---|---|---|---|
| Group | | | Dose | volume | Schedule of | | No. of |
| ID | Treatment | Vehicle | [mg/kg] | [ml/kg] | administration* | Route | mice |
| 1 | PBS | PB-saline | — | 10 | QDx5, followed by BIW | iv | 16 |
| | | | | | (Mon, Thu) x3 weeks | | |
| 2 | Sorafenib | — | 10 | 5 | BID x27 | po | 8 |
| 3 | miR-7-5p | NOV340 | 3 | 5 | QDx5, followed by BIW | iv | 8 |
| | | | | | (Mon, Thu) x3 weeks | | |
| 4 | miR-7-5p | Table 1 entry 1 | 3 | 10 | QDx5, followed by BIW | iv | 8 |
| | | | | | (Mon, Thu) x3 weeks | | |
| 5 | miR-7-5p | Table 1 entry 1 | 1 | 10 | QDx5, followed by BIW | iv | 8 |
| | | | | | (Mon, Thu) x3 weeks | | |
| 6 | miR-193a-3p | Table 1 entry 1 | 10 | 10 | QDx3, followed by BIW | iv | 8 |
| | | | | | (Mon, Thu) x3 weeks | | |
| 7 | miR-193a-3p | Table 1 entry 1 | 3 | 10 | QDx5, followed by BIW | iv | 8 |
| | | | | | (Mon, Thu) x3 weeks | | |
| 8 | miR-193a-3p | Table 1 entry 1 | 1 | 10 | QDx5, followed by BIW | iv | 8 |
| | | | | | (Mon, Thu) x3 weeks | | |

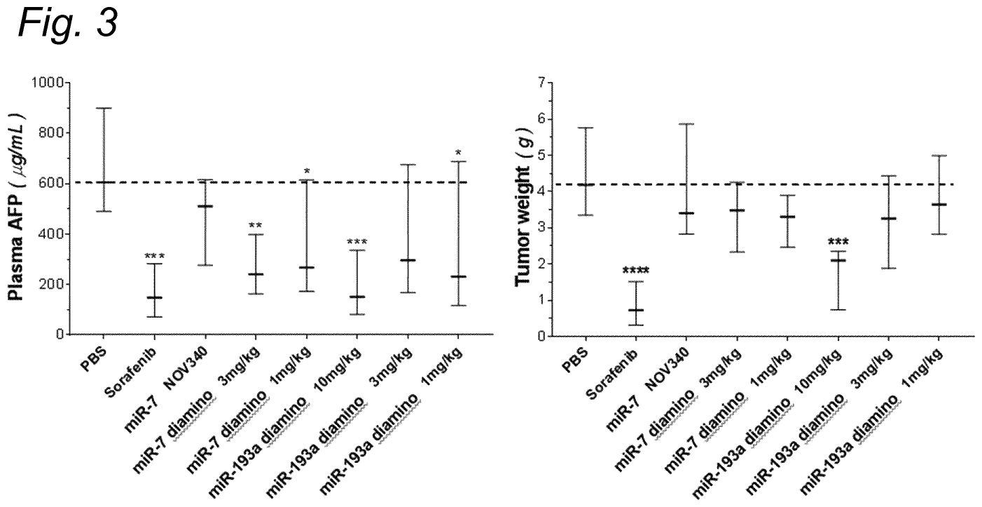

The day 42 plasma AFP levels and the tumor weights determined after terminal sacrifice are presented in FIG. 3.

### Conclusion:

miR-193a formulated in diamino lipid nanoparticles mediated significant TGI effects in the orthotopic mouse model of human Hep3b HCC (hepatocellular carcinoma) tumors, while miR-7 formulated in diamino lipid nanoparticles or in NOV340 showed very mild effects or no effect, respectively.

### Example 4.4: Tumor Growth Inhibitory Effects and Long-Term Immunity of miRNA-193a Formulated in Diamino Lipid Nanoparticles in a Syngeneic Mouse Model of 4T1 Triple Negative Breast Cancer Tumors Implanted in the Mammary Fat Pad

Six to eight weeks old female BALB/c mice (Shanghai Lingchang Bio-Technology Co. Ltd, Shanghai, China) were injected with 3×105 4T1 mouse tumor cells in PBS (0.1 mL/mouse) into the mammary fat pad. At the time of randomization, the TVs ranged between 69.9 and 173.9 mm3 (median 105.7, IQR=100.7-125.4). Body weights and TVs (caliper measurements) were determined 2-3 times/week. After randomization (day 9 after tumor inoculation) mice received different treatments under different dosing regimen (see Table 9 for dosing scheme). For all groups, 4 mice are sacrificed 48h after the 2nd miRNA-193a injection in the first week and in the 2nd week. Primary tumors were surgically removed from the mammary fat pad on day 20 after inoculation. After a recovery period of 3 days, treatment was resumed for 6 more weeks until day 63. Re-growth of (distant) tumors was monitored in addition to BW, and mice were sacrificed if end-points were met.

To investigate the long-term immunity in miRNA-193a treated mice against 4T1 cells, on day 75 after inoculation, mice treated with miRNA-193a and 8 naïve (non-tumor bearing) mice were re-challenged by subcutaneous injection of 3×105 4T1 mouse tumor cells in PBS (0.1 mL/mouse) into the right front flank. TV and BW were monitored for 3 weeks. Then, to investigate the immunization status of miRNA-193a treated mice against other cell types, on day 101, mice treated with miRNA-193a and 8 naïve (non-tumor bearing) mice were re-challenged by subcutaneous injection of H22 (mouse liver tumor cells) cells in PBS (0.1 mL/mouse) into the right lower flank.

TABLE 9
Dosing scheme example 4.4 - *BIW: twice a week; ip = intraperitoneal.

| | | | Dose | Schedule of | | No. of |
|---|---|---|---|---|---|---|
| Group | Treatment | Vehicle | (mg/kg) | administration* | Route | mice |
| 1 | PBS | PB-saline | 0 | BIW (Mon, Thu) x | iv | 12 |
| | | | | 6-8 weeks | | |
| 2 | miRNA-193a-3p | Table 1 entry 1 | 10 | BIW (Mon, Thu) x | iv | 12 |
| | | | | 6-8 weeks | | |
| 3 | Anti-PD-1 (RMP1-14) | — | 10 | BIW x 6-8 weeks | ip | 12 |
| 4 | miRNA-193a-3p + | Table 1 entry 1 +− | 10 + 10 | BIW (Mon, Thu) x 6-8 | iv + ip | 12 |
| | Anti-PD-1 (RMP1-14) | | | weeks; BIW x 6-8 weeks | | |

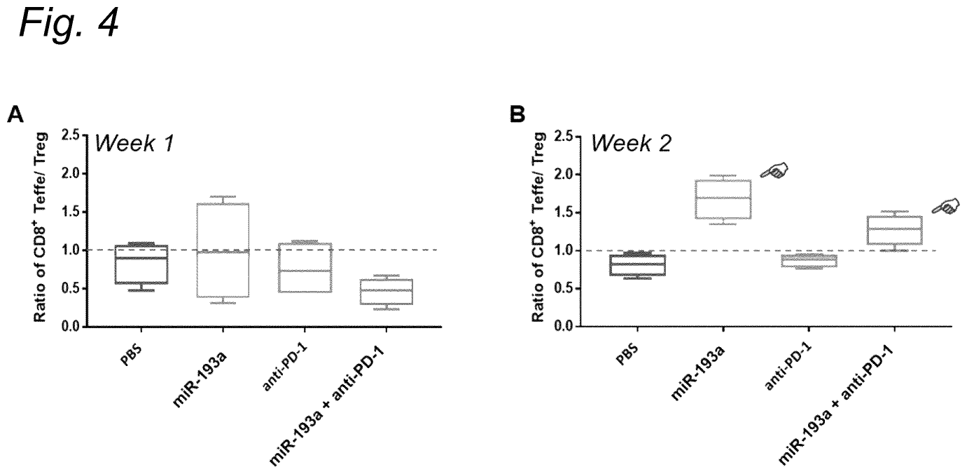

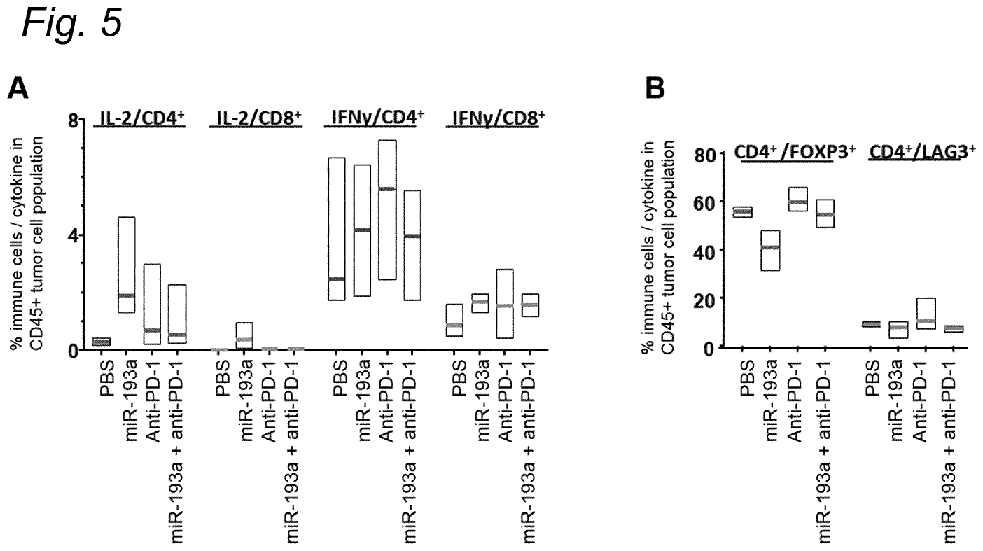

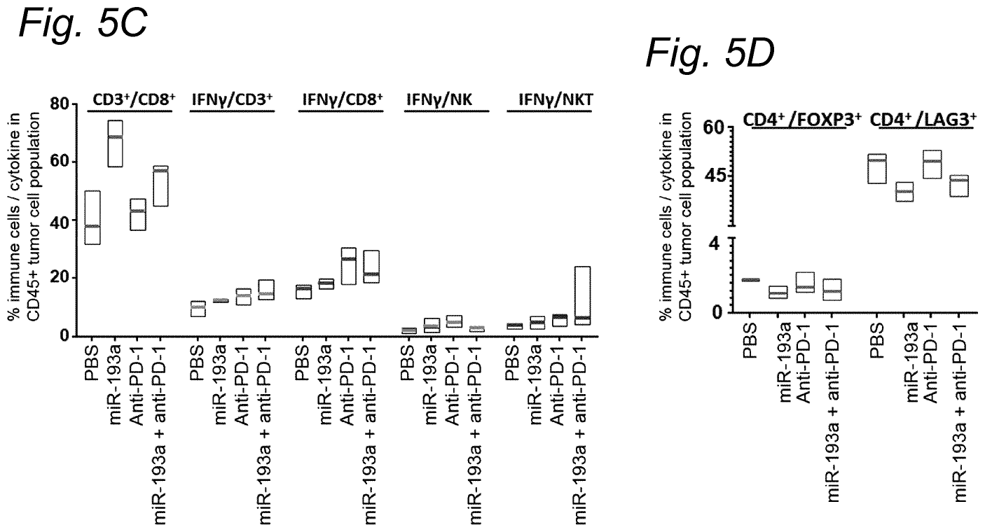

FIG. 4 shows the ratio of CD8+ T cells/Treg cells 1 (A) and 2 (B) weeks post treatment start. miRNA-193a treatment resulted in a shift from an immunosuppressive to an immunostimulatory 4T1 tumor microenvironment (CD8+ T cells/Treg cell >1, 2 weeks post start of miRNA-193a treatment). FIG. 5 shows the percentage of immune cells and intracellular cytokines in CD45+ tumor cell population. After 1 week, miRNA-193a treatment resulted in a significant increase in T-cell function (production of IFNγ and IL-2), and a significant decrease in regulatory T cell population (FOXP3+/LAG3+). After 2 weeks, miRNA-193a treatment resulted in a significant increase in T-cell frequency (CD8+) along with a mild induction in T-cell function (IFNγ) and a significant decrease in regulatory T cell population (FOXP3+/LAG3+).

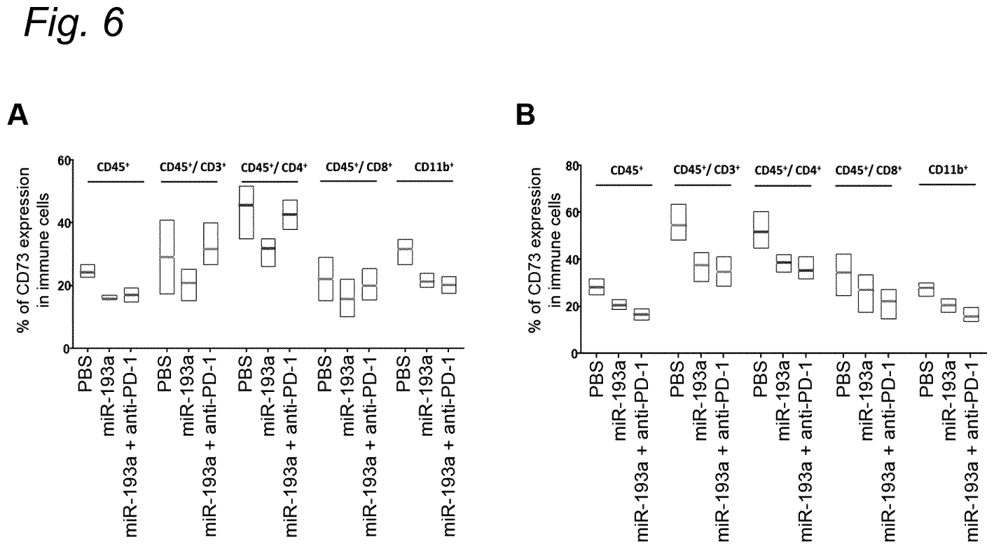

FIG. 6 shows the percentage of CD73 (NT5E) expression level in immune cells. Upon miRNA-193a treatment CD73 expression level is down-regulated in immune cells.

In conclusion, miRNA-193a treatment resulted in a shift from an immune-suppressive to an immune-stimulatory 4T1 tumor microenvironment by enhancing the T-cell function in the first week and induction of the T-cell frequency in the second week. This immune-oncology profile indicates that miRNA-193a is able to turn a cold tumor microenvironment to a hot tumor microenvironment.

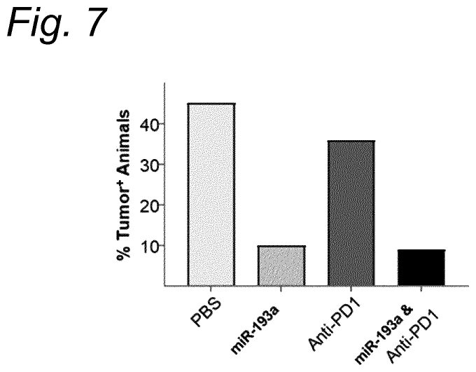

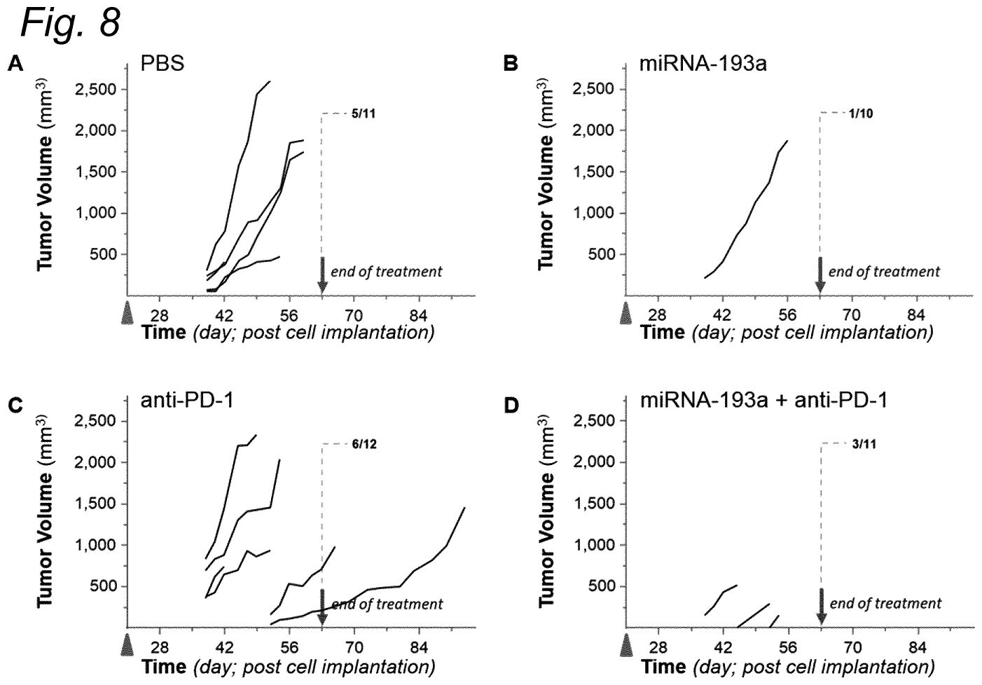

FIG. 7 shows the percentage of mice that show primary tumor regrowth post 4T1 tumor resection. Mice were injected with 4T1 cells in mammary fat pad, twice weekly treatment (i.v.) started 1 week post cell injection, primary tumor removal at day 20 post cell injection. After primary tumor removal mice were treated for a further 6 weeks at twice weekly schedule at 10 mg/kg of miR-193a formulated in diamino lipid nanoparticles, as compared to PBS or Anti-PD1 treated mice, or treated with a combination. FIG. 8 shows results for individual mice with primary tumor regrowth post 4T1 tumor resection. Conclusion

Treatment with miRNA-193a formulated in diamino lipid nanoparticles reduced tumor re-growth after tumor excision.

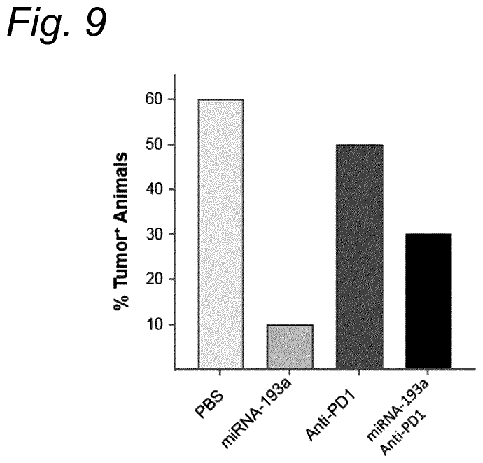

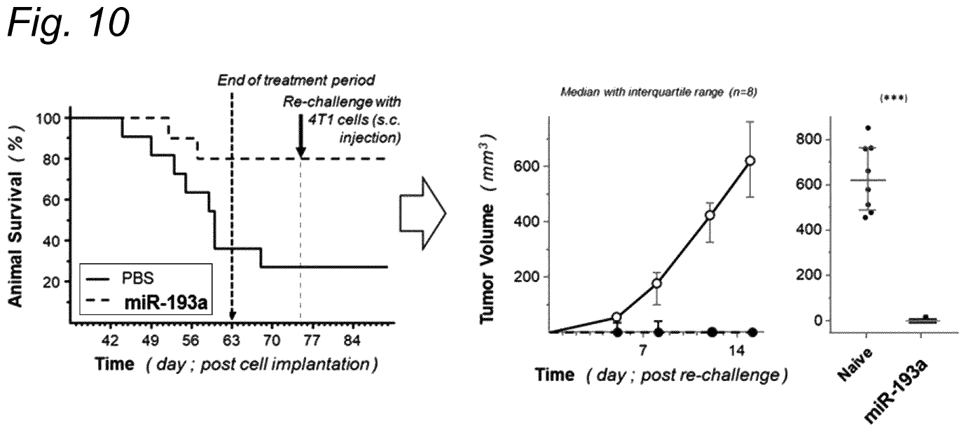

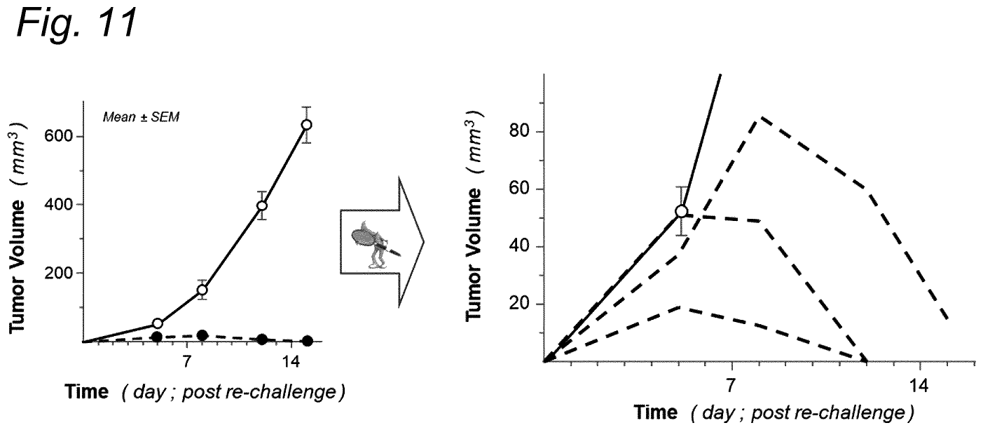

FIG. 9 shows the percentage of mice that show primary tumor regrowth post 4T1 tumor resection (Day 66). FIG. 10 shows results for when the surviving miRNA-193a treated mice were re-challenged with 4T1 cells. FIG. 11 shows detailed tumor volume (from FIG. 10) of the 3 miRNA-193a treated mice that showed tumor take compared to naïve mice when re-challenged with 4T1 cells.

### Conclusion:

Treatment with miRNA-193a formulated in diamino nanoparticles reduced tumor re-growth after both tumor excision and after a re-challenge with 4T1 tumors, and also positively affected mouse survival. As expected, re-grafted murine 4T1 cells were able to form subq tumors in naive animals. Pronounced prevention of tumor take/growth in miRNA-193a-treated animals strongly suggesting a long-term immunization against 4T1 cells.

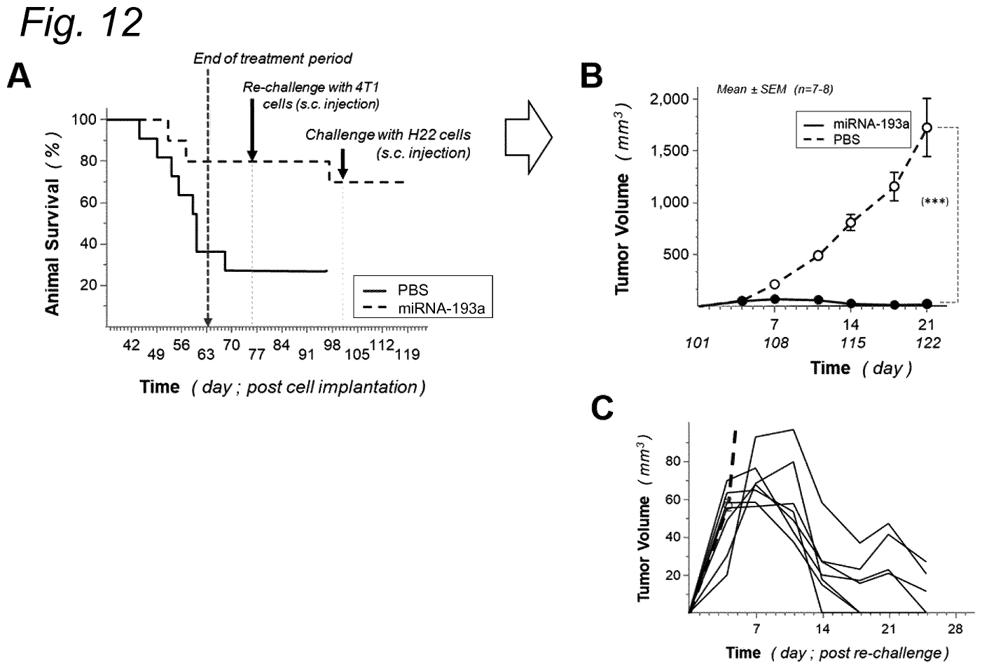

FIG. 12 shows how the surviving miRNA-193a treated mice were re-challenged with H22 cells on day 101, 38 days post end of treatment, and shows tumor volume as compared to naïve mice challenged with H22 cells. Detailed tumor volume of the miRNA-193a treated mice that showed tumor take (100%) compared to naïve mice when re-challenged with H22 cells, with pronounced time-dependent tumor regression after 1 week in all miRNA-193a treated animals.

### Conclusion

As expected, grafted murine H22 cells were able to form subq tumors in naive animals. Efficient (100%) tumor take occurred in miRNA-193a-treated animals, but rapid inhibition of H22 tumor growth was found, leading to time-dependent regression. This strongly suggest a long-term immunization against unrelated H22 cells (cross-antigen reaction).

Overall, treatment with miRNA-193a formulated in diamino lipid nanoparticles:
Reduced tumor re-growth after tumor excision, positively affecting mouse survival
Resulted in a shift from an immunosuppressive to an immunostimulatory 4T1 Tumor microenvironment
Pronounced prevention of tumor take/growth in miRNA-193a-treated animals strongly suggests long-term immunization against 4T1 cells (CD8+ T cells/Treg cell >1)
Efficient (100%) tumor take in miRNA-193a-treated animals, but rapid inhibition of H22 tumor growth, leading to time-dependent regression—suggesting long-term immunization against unrelated H22 cells (cross-antigen ‘vaccination’).

### Example 4.5: Tumor Growth Inhibitory Effects of Different Concentrations of miRNA-193a Formulated in Diamino Nanoparticles in Mice Bearing Subq Human A2058 Melanoma Tumors

Six to eight weeks old female BALB/c nude mice (Shanghai Lingchang Bio-Technology Co. Ltd, Shanghai, China) were unilaterally and subcutaneously injected in the right flank with 5×106 A2058 tumor cells in PBS (0.1 mL/mouse). At the time of randomization, the TVs ranged between 50.3 and 156.3 mm3 (median 104.8, IQR=91.9-127.1). After randomization, the mice received different miRNA-193a dosing concentrations under different dosing regimen (see Table 10 for dosing scheme). The BRAF-inhibitor vemurafenib was also included. Body weights and TVs (caliper measurements) were determined three times/week. *QDx3,QDx4=one injection per day for 3 or 4 consecutive days; BIW=twice a week, BID=twice daily. Po=per oral.

TABLE 10
dosing scheme for example 4.5

| | | | Dose | Dosing | Schedule of | | No. of |
|---|---|---|---|---|---|---|---|
| Group | Treatment | Vehicle | [mg/kg] | volume | administration* | Route | mice |
| 1 | PBS | — | — | 10 ml/kg | QDx4 (x3 weeks) | iv | 12 |
| 2 | miRNA-193a-3p | Table 1 entry 1 | 10 | 10 ml/kg | BIW | iv | 8 |
| | | | | | (Mon, Thu, x3 weeks) | | |
| 3 | miRNA-193a-3p | Table 1 entry 1 | 6.67 | 10 ml/kg | QDx3 (Mon, Tue, Wed) | iv | 8 |
| | | | | | (x3 weeks) | | |
| 4 | miRNA-193a-3p | Table 1 entry 1 | 5 | 10 ml/kg | QDx4 | iv | 8 |
| | | | | | (Mon, Tue, Wed, Thu) | | |
| | | | | | (x3 weeks) | | |
| 5 | Vemurafenib | — | 75 | — | BID (x 3 weeks) | po | 8 |

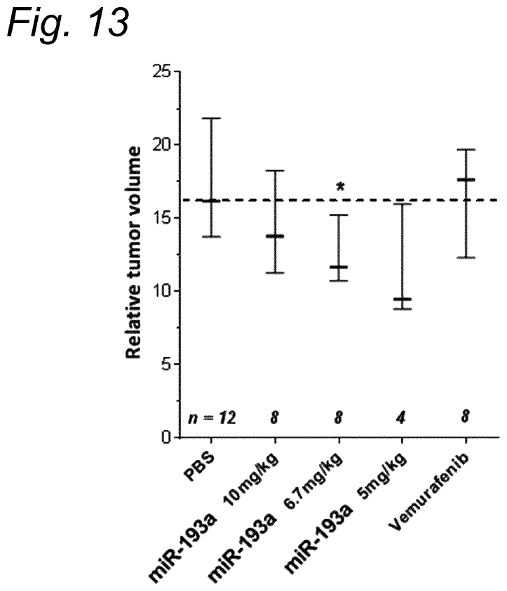

FIG. 13 shows relative tumor volume 21 days after the start of the treatment. Mice bearing subq human A2058 melanoma tumors were treated with different doses and different regimen of miRNA-193a formulated in diamino lipid nanoparticles, or with vemurafenib.

### Conclusion:

A significant reduction of tumor growth was observed for the QDx3 6.7 mg/kg miRNA-193a formulated in nanoparticles according to the invention, while the other dosing regimen only showed a mild trend

### Example 4.6: Investigating the PD Effect of miRNA-193a Treatment in Orthotopic 4T1 Murine Breast Cancer Syngeneic Model at Different Time Points

Six to eight weeks old female BALB/c mice (Shanghai Lingchang Bio-Technology Co. Ltd, Shanghai, China) were injected with 3×105 4T1 mouse tumor cells in PBS (0.1 mL/mouse) into the mammary fat pad. At the time of randomization, the tumor volumes (TVs) ranged between 252.30-370.45 mm³. After randomization mice received similar treatments with similar dosing regimen (see Table 11). Mice were sacrificed at pre-determined time points (see Table 11). The tumors from each mouse were collected, snap frozen in liquid nitrogen and then stored at −80° C.

TABLE 11
dosing scheme for example 4.6; QD is once a day; administration was iv

| | | | Dose | Time point | | Schedule of | No. of |
|---|---|---|---|---|---|---|---|
| Group | Treatment | Vehicle | [mg/kg] | (post final dose) | | administration* | mice |
| 1 | PBS | — | — | 2 | h | QDx2 | 4 |
| 2 | miRNA-193a-3p | Table 1 entry 1 | 10 | 2 | h | QDx2 | 4 |
| 3 | miRNA-193a-3p | Table 1 entry 1 | 10 | 4 | h | QDx2 | 4 |
| 4 | miRNA-193a-3p | Table 1 entry 1 | 10 | 8 | h | QDx2 | 4 |
| 5 | miRNA-193a-3p | Table 1 entry 1 | 10 | 24 | h | QDx2 | 4 |
| 6 | miRNA-193a-3p | Table 1 entry 1 | 10 | 48 | h | QDx2 | 4 |
| 7 | miRNA-193a-3p | Table 1 entry 1 | 10 | 72 | h | QDx2 | 4 |

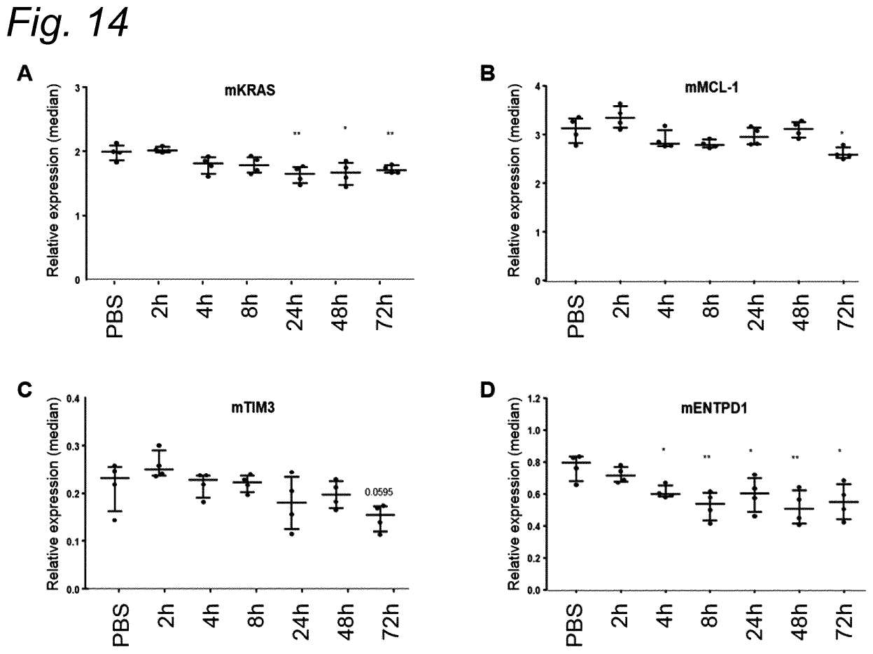

To investigate the effect of the miRNA-193a on mRNA expression levels of several important genes that were found to be target genes of miRNA-193a (pharmacodynamic effect), expression levels were quantified at different time points in tumors, using qPCR and primers described above. Assessed genes include K-Ras, MCL1, ENTPD1 (CD39) and TIM-3. The role and biological importance for these target genes is briefly discussed in Table 12. Results are shown in FIG. 14.

TABLE 12
summary of the role and the biological importance
of indicated miRNA-193a target genes.

| Target | Biological | Role in cancer | Role in immune |
|---|---|---|---|
| gene | importance | cell proliferation | system |
| K-RAS | Oncogene involved | Enhancing | No direct role |
| | in RAS pathway | proliferation | |
| MCL1 | Oncogene inducing | Enhancing | No direct role |
| | an anti-apoptosis | proliferation | |
| | response | | |
| ENTPD1 | Involved in | No direct role | Immune suppressive |
| (CD39) | adenosine genera- | | role by generating |
| | tion pathway | | high amount of |
| | | | adenosine |
| TIM-3 | Immune check point | No direct role | Inhibitory receptor |
| | receptor | | as immune sup- |
| | | | pressive marker |

FIG. 14 shows miRNA-193a target gene expression levels in the tumor over time after QDx2 (one injection per day, for two consecutive days) i.v. injection at 10 mg/kg. Mice bearing orthotopic 4T1 tumors were treated similarly and tumors were removed for pharmacodynamical analysis at different time points (see table 11). Individual tumor expression values are presented. Different target genes are significantly down-regulated at different time points.

### Conclusion

miRNA-193a treatment dosed at 10 mg/kg and at a QDx2 schedule resulted in a significant reduction in target mRNAs expression involved in apoptosis and the immune pathway at various time points.

### Example 4.7: Mode of Action of miRNA-193a in the In Vitro Settings Used

Different biological effects of miRNA-193a-3p have been tested in various cancer cell lines (see Table 13). For this, various cells were treated with miRNA-193a at different concentrations (1, 3, 10 nM). For all experiments controls (untreated, mock and scrambled) were measured. All assays were performed at 24h, 48h and 72h time points. Data shown in Table 13 are the results for miRNA-193a at 10 nM concentration at indicated time points. All the results have been quantified and normalized to the mock control. 10 nM has been shown to be a suitable concentration for miRNA-193a treatment in vitro, because cells show no signs of a toxic effect at that concentration.

TABLE 13
summary of miRN-193a in vitro modes of action

| | | | | Cell cycle | Motility |
|---|---|---|---|---|---|
| Cancer | Cell | Viability | Apoptosis | arrest | (18 to |
| type | line | (96 h) | (48/72 h) | (72 h) | 24 h) |
| HCC | HeP3B, | ↓↓ | ↑ | G2/M | ↓ |
| | SNU449 | | | | |
| HCC | Huh7 | ↓↓ | ↑ | — | n.a. |
| Melanoma | A2058 | ↓↓ | ↑↑ | G2/M | ↓ |
| NSCLC | A549, | ↓↓ | ↑↑ | SubG1 | ↓, — |
| | H460 | | | | |
| NSCLC | H1299 | ↓↓ | ↑ | — | n.a. |
| NSCLC | H1975 | ↓↓ | ↑ | G2/M | n.a. |
| TNBC | 4T1 | ↓ | ↑ | n.a. | ↓ |
| TNBC | EMT6 | ↓↓ | ↑↑ | n.a. | — |
| Pancreas | Panc-1 | ↓↓ | ↑↑ | G2/M | n.a. |
| Colon | HCT116 | ↓↓ | ↑↑ | — | n.a. |

↓↓ Viability < 50%
↓ Viability > 50%
↑↑ Induction > 2x
↑ Induction < 2x

miRNA-193a treatment in various cancer cell lines decreased cell viability over time as measured by either an MTS assay or by cell count. It enhanced apoptosis induction over time as measured by a caspase 3/7 apoptosis assay. Cell cycle arrest profiles were measured performing either nuclei imaging or flow cytometry. miRNA-193a treatment induced either a G2/M or a SubG1 cell cycle arrest profile in a manner depending on the cell line. While in Huh7, H1299, and HCT116 no obvious cell cycle arrest profile was observed following the indicated methods, an increased apoptosis was observed indicated by Caspase 3/7 activation and enhanced cleaved-parp protein on western blot (data not shown) following miRNA-193a treatment in these cell lines. This result indicates that miRNA-193a treatment affects the viability of cancer cells through induction of apoptosis. It is of note that due to intrinsic characteristics and gene mutation status of each cell line, performing one method to detect apoptosis is not always ideal for all cell lines. The cell motility of several cancer cell lines was significantly decreased upon miRNA-193a treatment as assessed via a Boyden chamber assay.

### Conclusion

miRNA-193a treatment on cancer cell lines decreases cell viability partly by inducing apoptosis and by an increase in the cell cycle arrest profile. miRNA-193a treatment also decreases cell motility of cancer cells, indicating its role in the inhibition of cancer cell migration.

### Example 4.8: miRNA-193a Affects the Adenosine Generation Pathway Partially Through Regulation of CD73 and CD39

Adenosine generation is one of the routes by which certain tumours evade host immunity. CD39 (ENTPD1) and CD73 (NT5E) are two cell surface ectoenzymes that dephosphorylate ATP to produce adenosine, thus controlling adenosine and ATP levels in the extracellular space. Extracellular adenosine has been shown to promote tumour growth and metastasis by limiting anti-tumour T-cell immunity. CD73 and CD39 are highly overexpressed on most tumour cells, leading to elevated levels of adenosine in the tumour microenvironment.

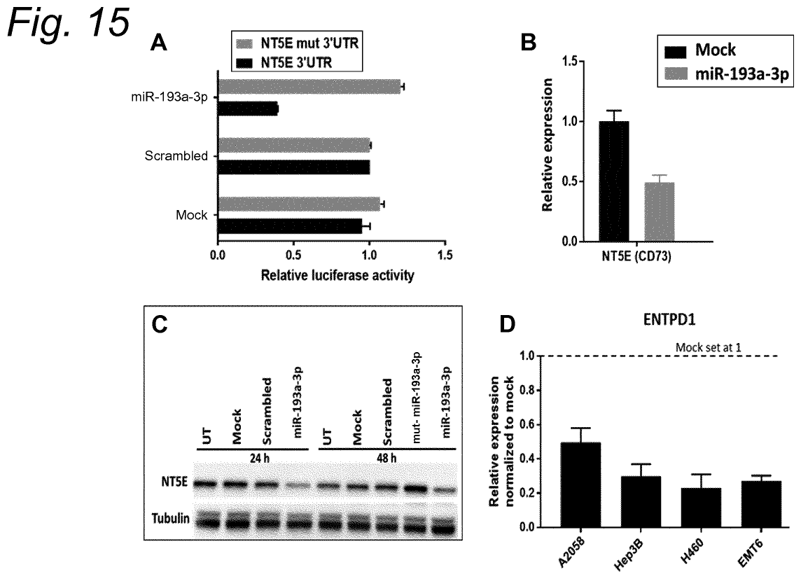

To validate CD73, as a target for miRNA-193a-3p, a 3′UTR assay was performed in which miRNA-193a-3p was overexpressed in Hela cells leading to downregulation of the activity of the reporter construct containing NT5E 3′UTR region compared with mock and scrambled controls. Whereas overexpression of miRNA-193a-3p did not affect the luciferase activity of the reporter construct containing mutated form of the CD73 3′UTR (FIG. 15A). This indicates that the 3′ UTR of CD73 is perfectly complementary with miRNA-193a-3p and CD73 is one of the validated targets for miRNA-193a-3p.

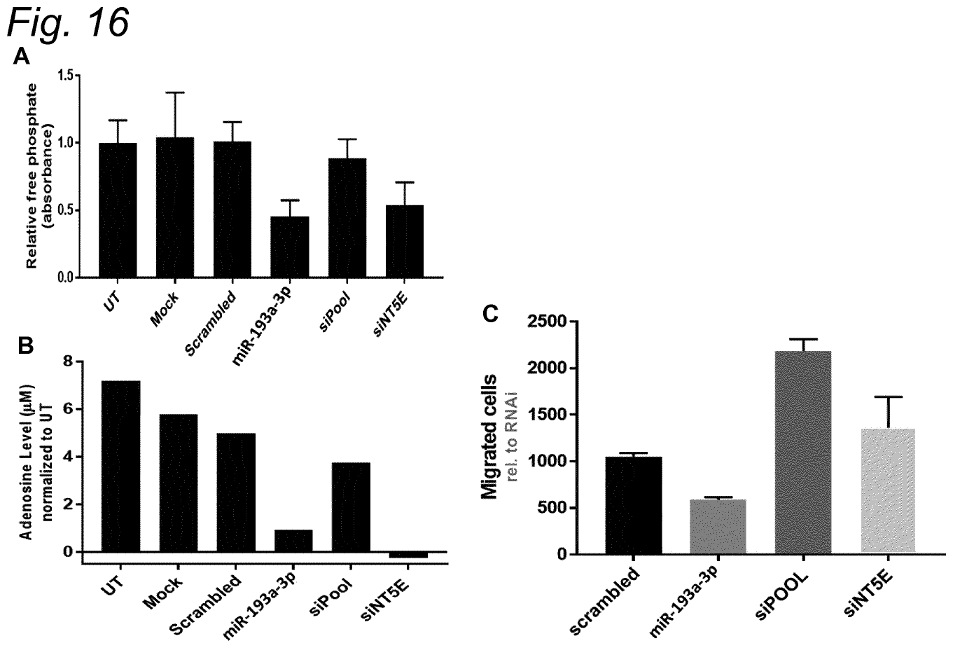

As shown in FIG. 15B-D, miRNA-193a-3p treatment downregulated the expression of both enzymes involved in adenosine generation pathway in a variety of cell lines at mRNA and protein level. To assess the impact of miRNA-193a-3p on adenosine generation, the release of free phosphate in A2058 melanoma cells was measured as a read out for ATP, ADP, and AMP dephosphorylation in supernatants. As illustrated in FIG. 16A, miRNA-193a-3p treatment reduced the level of free phosphate production. Similar results were found by measuring the direct amount of adenosine in cell culture supernatants (FIG. 16B). Further, the role of miRNA-193a in A2058 cancer cell migration was investigated. Using in vitro transwell assays, we showed that miRNA-193a-3p treatment significantly suppressed migration ability of A2058 cells (FIG. 16C). Interestingly, siRNA mediated depletion of NT5E phenocopied the effect of miRNA-193a treatment in all these experiments, strongly suggesting that miRNA-193a may exert its function on adenosine generation and migration at least partially via targeting NT5E (FIGS. 16A, 16B and 16C).

### Conclusion

miRNA-193a plays a role in downregulating the immunosuppressive tumor microenvironment partially through targeting NT5E and ENTPD1 and inhibition of adenosine generation. miRNA-193a also partially reduces the adenosine-induced migration ability of cancer cells through targeting NT5E.

### Example 4.9: Cell Cycle Distribution Upon miRNA-193a Treatment in Different Cell Lines at Optimal Time Point

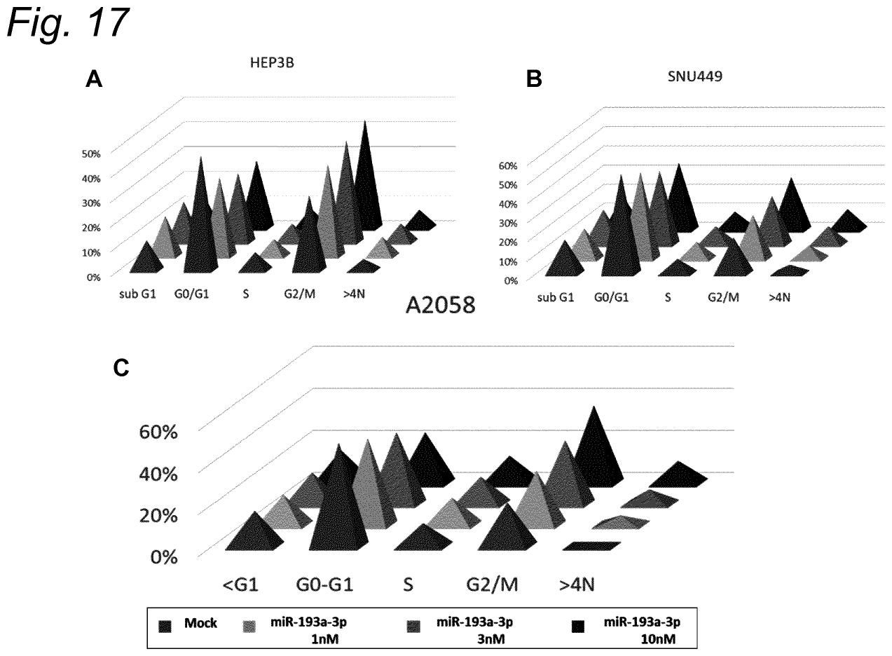

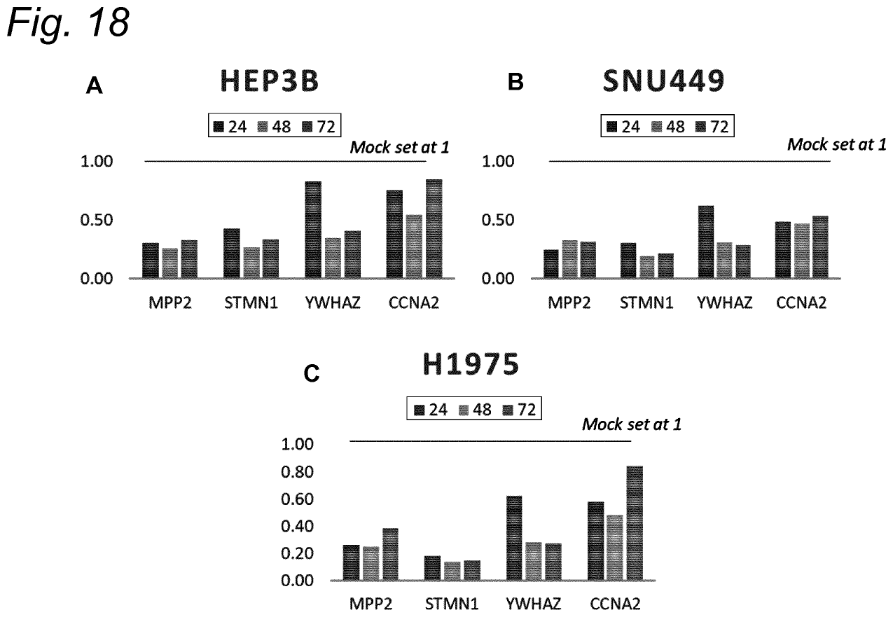

To investigate the anti-proliferative property of miRNA-193a we profiled the cell cycle status of different cancer cells upon mi RNA-193a-3p treatment at different concentrations (1 nM, 3 nM and 10 nM) and time points (48h, 72h and 96h) compared to mock as control. miRNA-193a-3p treatment led to a G2/M arrest phenotype in HCC cell lines of Hep3B and SNU449 and melanoma A2058 cells (FIG. 17) which eventually resulted in cell death (data not shown). Similar phenotype has been observed in Panc1 (pancreatic cancer cell) as well as H1975 (lung cancer cell) (data not shown). To partially address the miRNA-193a-dependent G2/M arrest phenotype, the expression level of several miRNA-193a target genes that can play a role in G2/M arrest were investigated at different time points (24h, 48h and 72h) and shown to be down-regulated (FIG. 18) in Hep3B, SNU449 and H1975 cancer cells. The expression level of these genes is also down-regulated in other cell lines in which a G2/M arrest phenotype was shown (data not shown). MPP2 and STMN1 associate with cytoskeleton and therefore regulate cell division and proliferation at the G2/M phase, while YWHAZ and CCNA2 play roles in regulation of G2/M phase checkpoint by binding and sequestering the cyclin-dependent kinases.

### Conclusion

In all tested cancer cell lines expression of miRNA-193a triggers cancer cell death at least partially due to its effect on inducing a G2/M arrest phenotype and stopping cell division. This phenotype is partially caused by the microRNA drug inhibiting genes associated with cytoskeleton and cell division.

### Example 4.10: RNA Sequencing, Gene Set Enrichment Analysis, and Pathway Analysis Upon Treatment of miRNA-193a in 6 Different Cancer Cell Lines

Implementation of high-throughput RNA-sequencing has become a powerful tool for comprehensive characterization of the whole transcriptome at gene and exon levels and with a unique ability to identify differentially expressed genes, novel genes and transcripts at high resolution and efficiency. However, till date, very few miRNAs have been characterized for their specific role in cancer development. Hence, we have used the high-throughput RNA-sequencing after overexpressing miRNA-193a-3p in 6 different cancer cell lines including A540 and H460 (both lung cancer), Huh7 and Hep3B (both liver cancer) A2058 (skin cancer) and BT549 (breast cancer) at 24h post miRNA-193a-3p treatment at 10 nM. The gene expression was compared to mock as control and we identified differentially expressed genes and their biological pathways.

Lists of downregulated genes (relative expression miRNA-193a-3p/relative expression mock <1) at 24h after transfection were created for all six cell lines. Subsequently, we generated lists of genes that were significantly downregulated (adjusted P<0.1) in at least one cell line or in multiple cell lines (Table 14). More than 65% of genes downregulated in at least two cell lines were predicted miRNA-193a-3p targets according to the TargetScan tool (a miRNA target-prediction tool).

TABLE 14
Number of genes down-regulated by miRNA-193a
in at least one or up to six cell lines

| # | Down-regulated | Adjusted P value < 0.1 | % |
|---|---|---|---|
| cell lines | genes | Predicted targets | Predicted |
| =6 | 35 | 9 | 83 |
| ≥5 | 181 | 114 | 63 |
| ≥4 | 218 | 144 | 66 |
| ≥3 | 242 | 161 | 67 |
| ≥2 | 282 | 183 | 65 |
| ≥1 | 352 | 203 | 58 |

miRNA-193a downregulated 35 genes in all six cell lines (Table 11) considering the adjusted P<0.1, which are expected to play roles in regulation of apoptosis, cell migration, adhesion, proliferation, and other oncogenic functions.

TABLE 11
Genes downregulated in all six cell lines.

| Gene | Full name |
|---|---|
| FOXRED2 | FAD dependent oxidoreductase domain containing 2 |
| ERMP1 | Endoplasmic reticulum metallopeptidase 1 |
| NT5E | 5′-nucleotidase ecto |
| SHMT2 | Serine hydroxymethyltransferase 2 |
| HYOU1 | Hypoxia up-regulated 1 |
| TWISTNB | TWIST neighbor |
| AP2M1 | Adaptor related protein complex 2 subunit mu 1 |
| CLSTN1 | Calsyntenin 1 |
| TNFRSF21 | TNF receptor superfamily member 21 |
| DAZAP2 | DAZ associated protein 2 |
| C1QBP | Complement C1q binding protein |
| STARD7 | StAR related lipid transfer domain containing 7 |
| ATP5SL | Distal Membrane Arm Assembly Complex 2 |
| DCAF7 | DDB1 and CUL4 associated factor 7 |
| DHCR24 | 24-dehydrocholesterol reductase |
| DPY19L1 | Dpy-19 like C-mannosyltransferase 1 |
| AGPAT1 | 1-acylglycerol-3-phosphate O-acyltransferase 1 |
| SLC30A7 | Solute carrier family 30 member 7 |
| AIMP2 | Aminoacyl tRNA synthetase complex interacting |
| | multifunctional protein 2 |
| UBP1 | Upstream binding protein 1 |
| RUSC1 | RUN and SH3 domain containing 1 |
| DCTN5 | Dynactin subunit 5 |
| ATP5F1 | ATP synthase peripheral stalk-membrane subunit b |
| CCDC28A | Coiled-coil domain containing 28A |
| SLC35D2 | Solute carrier family 35 member D2 |
| WSB2 | WD repeat and SOCS box containing 2 |
| SEC61A1 | Sec61 translocon alpha 1 subunit |
| MPP2 | Membrane palmitoylated protein 2 |
| FAM60A | SIN3-HDAC Complex Associated Factor |
| PITPNB | phosphatidylinositol transfer protein beta |
| POLE3 | DNA polymerase epsilon 3, accessory subunit |

For clustering and pathway analysis, a bigger gene set was required. We therefore used 242 genes that were downregulated in at least three cell lines (adjusted p<0.1) as input for the DAVID functional classification tool, which allows genes to be clustered into functionally related groups (Table 16). This analysis showed that the most enriched cluster included genes that regulate apoptosis. Other clusters contained genes with roles in angiogenesis, unfolded protein response, chemotaxis, protein transport, nucleoside metabolism, glycosylation, oncogenesis, wound healing. Interestingly, genes that regulate immune activation were also affected by miRNA-193a. Among the 242 significant genes downregulated in at least 3 cancer cell lines (adjusted P<0.1), 161 genes are of interest in the context of miRNA-193a-3p as they have also been predicted by different target prediction programs to be a target for miRNA-193a-3p. These 161 genes are ERMP1, MCL1, ZDHHC18, KIAA1147, IDS, EIF4B, ETS1, TXLNA, NT5E, WSB2, PLAUR, LRRC40, PTPLB, SLC15A1, NCEH1, IL17RD, STMN1, AIMP2, PHACTR2, GALNT1, LAMC2, SCP2, SLC26A2, LUZP1, SHMT2, UBP1, PHLDA2, ST5, ENDOD1, CGNL1, MARCKSL1, RAB11FIP5, CCND1, RUSC1, FAM168B, ZC3H7B, PPTC7, SLC39A5, ACSS2, TPP1, HYOU1, DCTN5, CRKL, WDFY2, WDR82, SLC6A12, CDK6, SULF2, TWISTNB, ATP5F1, ALDH9A1, TOR4A, NET1, RSF1, NUP50, ZMAT3, AP2M1, MPP2, ITGB3, GALNT14, SLC35D1, PPARGC1A, TBL1XR1, MSANTD3, CLSTN1, PITPNB, CECR2, KIAA1644, ARHGAP29, KIAA1191, GREB1, PIK3R1, TNFRSF21, SEPN1, SYNRG, LRP4, ZNF365, CRYAA, MED21, GNAI3, ATP5SL, UBE2L6, ANKRD13A, GCH1, NIPA1, TRIM62, USP39, HEG1, DCAF7, LRRC8A, SOX5, IRF1, MORC4, UNC119B, FAF2, SLC30A1, C1QBP, ST3GAL4, TRIB2, TBC1D5, TMPPE, MAPK8, CBX1, CADM1, CCDC28A, YWHAZ, ERAP2, STON2, AP5M1, TGFB2, CYTH1, FAM20B, DPY19L1, ARHGAP19, LPAR3, LMLN, NUDT15, PLAU, VAMP8, HHAT, AGPAT1, ATP8B2, ACPL2, SCAMP4, LAT2, OSMR, NUDT21, HPRT1, STARD7, GABPA, CDC42EP2, THBS4, ATP6V1B2, PRNP, GFPT1, MAX, KRAS, CNOT6, NUDT3, RFWD3, APPL1, SLC23A2, BOD1, PDE3A, SLC30A7, CEP41, NOTCH2, RGS2, CDC42EP4, TP53INP1, SQSTM1, DDAH1, SLC35D2, FOCAD, GPATCH11, CBL, TMEM30B, HFE, PLEKHB2, ARPC5, and ABI2. Among these genes, NT5E, TNFRSF21, YWHAZ, MAPK8, PLAU, PLAUR, NOTCH2, ETS1, IL17RD, CDK6, EIF4B, and MCL1 are particularly interesting for their crucial involvement in the cell cycle pathway, in immune activation, as well as in cell movement. Among all the genes that have been significantly down-regulated (P<0.05), CDK4, CDK6, CRKL, NT5E, HMGB1, IL17RD, KRAS, KIT, HDAC3, RTK2, TGFB2, TNFRSF21, PLAU, NOTCH1, NOTCH2, and YAP1 are particularly interesting for their known involvement in anti-tumor immunity. ETS1, YWHAZ, MPP2, PLAU, CDK4, CDK6, EIF4B, RAD51, CCNA2, STMN1, and DCAF7 are of particular interest for their crucial involvement in regulation of the cell cycle.

TABLE 16
Top ten pathways regulated by miRNA-193a-3p. Annotation clustering was performed using
DAVID software. The most enriched functional clusters and their genes are presented.

| | Enrichment | |
|---|---|---|
| Gene cluster | score (ES) | Genes |
| Apoptosis | 2.55 | KCNMA1, NOTCH2, TNFRSF21, YWHAZ, CADM1, CRYAA, ETS1, |
| | | AIMP2, SQSTM1, ZMAT3, TGM2, CECR2, PDE3A, STRADB, |
| | | NIPA1, MAPK8, TP53INP1, PRNP, PRT1, GCH1, DHCR24, |
| | | TGFB2, NET1, PHLDA2, TPP1 |
| Angiogenesis | 2.17 | CRKL, CTGF, ZMIZ1, TGM2, ELK3, LOX, UBP1, PLAU, CYR61, |
| | | TGFB2 |
| Unfolded | 1.84 | ERMP1, NCEH1, SEC31A, CLSTN1, FOXRED2, SEPN1, EXTL2, |
| protein | | HYOU1, SLC35D1, SULF2, PTPLB, HHAT, ERAP2, FAF2, DPM3, |
| response | | PDZD2, SEC61A1, DHCR24, IDS, MOSPD2, DPM, PRNP, |
| | | AGPAT1 |
| Chemotaxis | 1.66 | CXCL1, RAC2, CXCL5, CYR61, PLAUR, KCNMA1, ABI2, HPRT1 |
| Protein | 1.51 | STON2, RAB11FIP5, SRP54, YWHAZ, SYNRG, GCH1, THBS4, |
| transport | | SRP54, TOMM20, SEC31A, TPP1, SLC30A7, TGFB2, AKAP12, |
| | | AP2M1, ITGB3, GNAI3, SORL1, KRAS, SLC15A1, SEC61A1, |
| | | APPL1, LRP4, PLEKHA8, STRADB, SCAMP4, HFE, CADM1, |
| | | ZMAT3, ARF3, VAMP8, NUP50, DHCR24, RAB11FIP5, ATP6V1B2, |
| | | SQSTM1, WNK4 |
| Nucleoside | 1.49 | NUDT3, NUDT15, NUDT21, DERA, NT5E, GCH1, HPRT1 |
| metabolism | | |
| Glycosylation | 1.47 | GALNT1, SLC35D1, ST3GAL5, SULF2, LAT2, GALNT1, NCEH1, |
| | | ST3GAL4, CHST14, B3GNT3, DPM3, GALNT13, DHCR24, |
| | | NUDT15, IDH2, PPTC7, HPRT1, EXTL2, SEC61A1, ERAP2, |
| | | GALNT14 |
| Oncogenesis | 1.37 | CCND1, CBL, CXCL1, CRKL, MAX, KCNMA1, TBL1XR1, GNAI3, |
| | | YWHAZ, RAC2, ETS1, PTCH1, MAPK8, LAMC2, PIK3R1, CRKL, |
| | | CDK6, CBL, APPL1, GNAI3, PDE3A, TGFB2, ABI2, MAX, ITGB3, |
| | | LOX, CXCL5, ARPC5, PPARGC1A, THBS4 |
| Wound | 1.18 | NOTCH2, KCNMA1, CXCL1, ITGB3, PLAU, CCND1, ZMIZ1, ELK3, |
| healing | | YWHAZ, IL11, PLAUR, LOX, CTGF, TGFB2 |
| Immune | 1.16 | NOTCH2, LAT2, LRRC8A, CRKL, LRRC8A, YWHAZ, PIK3R1, |
| activation | | IRF1, TGFB2, IL11, UNG, CDK6, HPRT1 |

### Conclusion:

Overexpression of miR-193a in 6 different cancer cell lines resulted in inhibition of a variety of targets affecting different pathways. While there were some common genes being significantly targeted in all 6 different cancer cells, there were also unique genes that were only targeted in each cell line, indicating a context-dependent effect. Pathway enrichment analysis on the genes that have been targeted in at least 3 different cancer cell lines significantly shows the gene signatures of angiogenesis, unfolded protein response, chemotaxis, protein transport, nucleoside metabolism, glycosylation, oncogenesis, wound healing, and immune activation. These data suggest that miR-193a is a crucial modulator in tumor progression and due to its ability to target multiple pathways, its therapeutic potential as anti-cancer drug is attractive.

### Example 4.11: T-Cell Mediated Immunity of miRNA-193a Formulated in Diamino Lipid Nanoparticles in a Syngeneic Mouse Model of 4T1 Triple Negative Breast Cancer Tumors Implanted in the Mammary Fat Pad

To investigate the T-cell mediated long-term immunity in miRNA-193a treated mice against 4T1 cancer cells, a new study with similar conditions to example 4.4 was performed. 5 days post-tumor inoculation, mice were randomized into 2 groups and received a similar treatment and dosing regimen as in example 4.4 (see Table 9, groups 1-2 only). After surgical removal of primary tumors when the group mean tumor volume reached 800 mm3, treatments were resumed and continued until day 58.

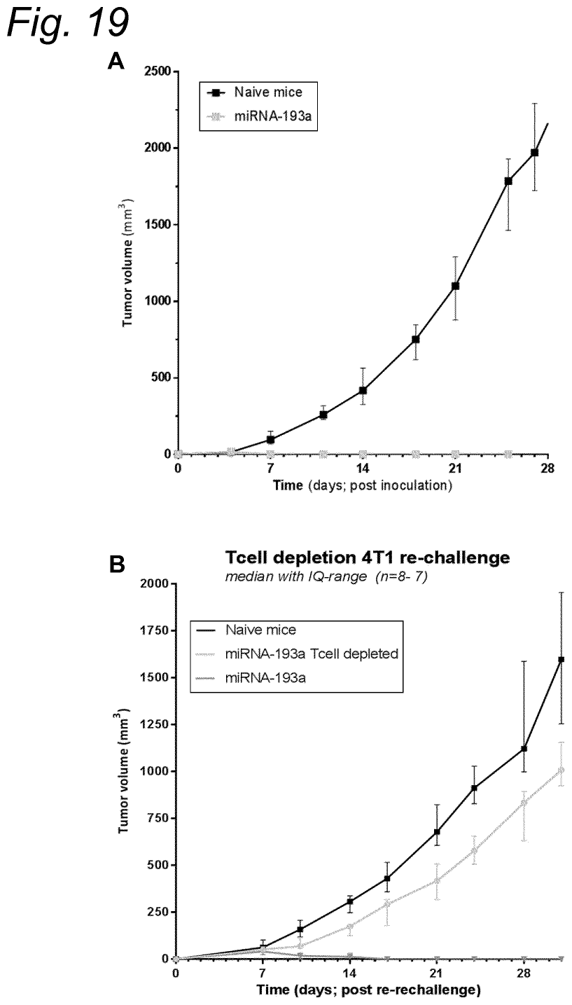

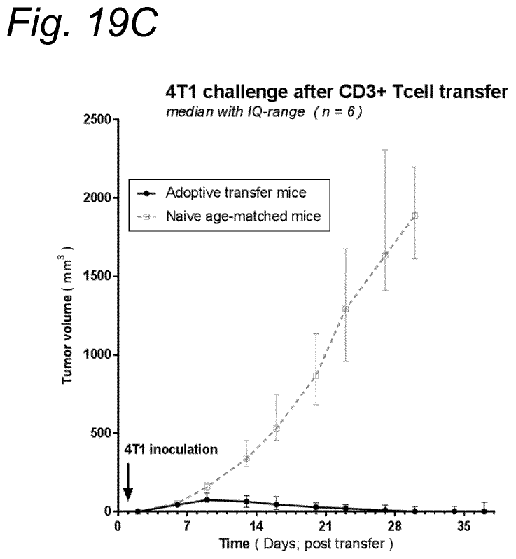

Due to lack of tumor re-growth in mice treated with miRNA-193a compared with PBS control (not shown for this study, similar data shown for example 4.4 in FIG. 8B), we re-investigated the long-term immunity in miRNA-193a treated mice against 4T1 cells. To do so, on day 76 post-tumor inoculation mice treated with miRNA-193a and naïve (age-matched non-tumor bearing) mice were re-challenged by 4T1 mouse tumor cells. Tumor re-growth after the re-challenge was followed up to 3 weeks. Mice previously treated with miRNA-193a did not develop any palpable tumor compared to the naïve mice (FIG. 19). To investigate our hypothesis on T-cell mediated long-term immunity in these mice, on day 103 post-tumor cell inoculation, 4T1 re-challenged mice previously treated with miRNA-193a and naïve mice were depleted for T-cells upon treatment with anti-CD4 and anti-CD8 antibodies (see Table 17 for treatment schedule). FACS analysis on the blood samples from mice in all groups has confirmed the results for T cell depletion. CD8+ cells showed a complete depletion and CD4+ cells showed a partial depletion (data not shown). 5 days after depletion treatment, mice in all groups were re-challenged again with 4T1 mouse tumor cells (3×105 in 0.1 mL PBS in front flank) and tumor growth was followed up to 4 weeks. Interestingly, similar to naïve mice, T cell depletion in mice previously treated with miRNA-193a resulted in 4T1 tumor growth, while mice previously treated with miRNA-193a that were not depleted for T cells did not show a palpable 4T1 tumor (FIG. 19).

To further confirm the T-cell dependent long-term immunity in mice previously treated with miRNA-193a, a T-cell transfer from the survivor mice (Table 17, group 2b) that did not develop a palpable tumor in age-matched naïve mice was performed. On day 133 post-tumor cell inoculation CD3+ T-cells were recovered from the spleens, auxiliary, brachial, and inguinal lymph nodes of the surviving animals. T-cells were pooled and transferred i.v. to 6 naïve age-matched mice (1×107 CD3+ Tcells per mouse at day 0). One day post-T-cell transfer, animals were re-challenged with 3×105 4T1 cells in PBS (0.1 mL/mouse) in the right mammary fat pad of 6 naive age-matched mice (as control group) and the 6 mice that received CD3+ T-cells. Tumor growth was followed for about 5 weeks. Interestingly, naive mice that received T-cells did not show any 4T1 tumor growth compared to control naïve mice (FIG. 19).

TABLE 17
Dosing scheme example 4.11 - *BIW: twice a week; ip = intraperitoneal,
QOD= one injection every other day, Q3D= one injection every 3 days

| | | | | Dose schedule/ | | Prior therapy at |
|---|---|---|---|---|---|---|
| Group | n | Treatment | Dose level | administration | Mice | efficacy stage |
| 1 | 8 | NA | | | Age matched | No treatment |
| | | | | | naïve mice | |
| 2a | 8 | Anti-CD4 + Anti-CD8 | 250 μg/mouse + | 1^{st} week: QOD x 3 doses | 4T1 survivors | miRNA-193a-3p |
| | | | 250 μg/mouse | 2^{nd}-4^{th} weeks: Q3D (i.p.) | | BIW x 8 weeks |
| | | | | | | (From day 5), i.v. |

| | | |
|---|---|---|
| 2b | 7 | NA |

### Conclusion:

Treatment with miRNA-193a (in this case miRNA-193a-3p formulated in diamino lipid nanoparticles) reduced tumor growth after a re-challenge with 4T1 tumor cells and enhanced mouse survival. As expected, re-grafted murine 4T1 cells were able to form subq tumors in naive animals. Pronounced prevention of tumor take/growth in miRNA-193a-treated animals strongly suggested a long-term immunization against 4T1. Previously miRNA193a treated mice which were the survivors after re-grafted by murine 4T1 cells, showed tumor re-growth only upon T-cell depletion compared to their non-depleted group. This result strongly indicates a T-cell dependent immunization. Further, T-cells transfer from previously miRNA-193a treated re-challenged survivor mice into naïve mice abrogated tumor re-growth after a re-challenge with 4T1 tumors. This strongly suggests a T-cell mediated immunity in miRNA-193a treated mice.

### Example 4.12: Efficacy of miRNA-193a Formulated in Diamino Lipid Nanoparticles on Primary Tumor Growth in 12 Syngeneic Tumor Models

In this study, the effect of miRNA-193a formulated in diamino lipid nanoparticles was investigated on the primary tumor growth in a panel of twelve syngeneic tumor models. Six to eight weeks mice (Shanghai Lingchang Bio-Technology Co. Ltd, Shanghai, China) were subcutaneously injected with an appropriate number of syngeneic cancer cells, depending on the cancer model (see Table 18). At the time of randomization, tumor volume (TV) was approximately 80-120 mm³. Body weights and TVs (caliper measurements) were determined 2-3 times/week. After randomization (indicated as day 0) mice received PBS or miR-193a (formulated in diamino lipid nanoparticles) treatments as shown in Table 19. Each tumor model had two groups as depicted in Table 19. Mice were scheduled to be euthanized after two weeks of follow up after a maximum of four weeks of treatment. However, depending on tumor growth rates of various treatments and experimental tumor models, some mice were euthanized earlier than planned at humane endpoint (when the mean TV/group reached 2000 mm3 or an individual mouse showed a TV of 3000 mm3). In the H22, Pan02, B16-BL6, RM-1, B16F10, MC38, A20, and EMT-6 models, miRNA-193a, significantly induced tumor growth inhibition (TGI). In the CT26, Renca, and Hepa1-6 models, miR-193a treatment did not induce significant TGI (Table 20). In table 20, percentage Tumor Growth Inhibition (TGI) by miRNA-193a compared to PBS was calculated using the median tumor volume at the latest comparable timepoint between treatment groups.

TABLE 18
Inoculation details for each cell line

| | | Cancer | | | | |
|---|---|---|---|---|---|---|
| No. | Cell line | Type | Cells | Inoculation site | Mouse Strain | Sex |
| 1 | CT26 | Colon | 5 × 10e5 | Right lower flank | BALB/c | Female |
| 2 | H22 | Liver | 1 × 10e6 | Right front flank | BALB/c | Female |
| 3 | Pan02 | Pancreatic | 3 × 10e6 | right front flank | C57BL/6J | Female |
| 4 | B16BL6 | Melanoma | 2 × 10e5 | right lower flank | C57BL/6J | Female |
| 5 | RM-1 | Prostate | 1 × 10e6 | right lower flank | C57BL/6J | Male |
| 6 | Renca | Kidney | 1 × 10e6 | right lower flank | BALB/c | Female |
| 7 | B16F10 | Melanoma | 2 × 10e5 | right lower flank | C57BL/6J | Female |
| 8 | MC38 | Colon | 1 × 10e6 | right lower flank | C57BL/6J | Female |
| 9 | Hepa 1-6 | Liver | 5 × 10e6 | right front flank | C57BL/6J | Female |
| 10 | LL/2 | Lung | 3 × 10e5 | right lower flank | C57BL/6J | Female |
| 11 | A20 | Lymphoma | 5 × 10e5 | right lower flank | BALB/c | Female |
| 12 | EMT-6 | Breast | 5 × 10e5 | right front flank | BALB/c | Female |

TABLE 29
Experimental groups for each model.

| | | | | | Dosing | | Dosing | | Dosing Frequency & |
|---|---|---|---|---|---|---|---|---|---|
| Group | N | Treatment | Dose | | Solution | | Volume | ROA | Duration |
| 1 | 10 | PBS | 0 | mg/kg | 0 | mg/ml | 10 μL/g | i.p. | BIW x up to 3 weeks |
| 2 | 10 | miR-193a | 10 | mg/kg | 1.0 | mg/ml | 10 μL/g | i.v | BIW x 3-4 weeks* |

TABLE 20
Tumor growth inhibition in syngeneic models by miRNA-193a

| | | % TGI by |
|---|---|---|
| Cell line | Cancer type | miRNA-193a |
| MC38 | Colon carcinoma | 71 |
| LL/2 | Lung carcinoma | 64 |
| B16-F10 | Melanoma | 56 |
| A20 | Reticulum cell sarcoma (lymphoma) | 54 |
| Pan02 | Pancreatic adenocarcinoma | 46 |
| H22 | Hepatocarcinoma | 45 |
| EMT-6 | Breast mammary carcinoma | 42 |
| B16-BL6 | Melanoma | 34 |
| RM-1 | Prostate cancer | 25 |
| Renca | Renal adenocarcinoma | 22 |
| CT26 | Colon carcinoma | 12 |
| Hepa 1-6 | Hepatoma | 7 |

### Conclusions:

miRNA-193a treatment resulted in significant tumor growth inhibition on the primary tumors in a wide range of syngeneic tumor models (i.e. H22, Pan02, B16-BL6, RM-1, B16-F10, MC38, A20, and EMT-6). These results suggest that miRNA-193a has a suppressor effect on the growth of established subcutaneous primary tumors in a wide range of syngeneic models.

### Example 4.13: miRNAs Formulated in Other Lipid Nanoparticles do not Inhibit Tumor Growth in Mice Bearing Orthotopic Human Hep3b Hepatocellular Carcinoma Tumors

Six to eight weeks old female SCID/Beige mice (Shanghai Lingchang Bio-Technology Co. Ltd, Shanghai, China) were orthotopically implanted with a single 2×2×2 mm piece of a subq grown Hep3b tumor into the left liver lobe. Mice were randomized based on day 21 AFP levels. At the time of randomization, the AFP levels (ng/ml plasma) ranged between 401 and 40628 ng/ml (median 2536, IQR=1037-5510). Treatments were with sorafenib, with vehicle control, or with various miRNAs (or scrambled miRNA control) encapsulated in NO340 lipid nanoparticles (Simonson and Das, Mini Rev Med Chem, 2015, 15(6): 467-474, PMID: 25807941). Treatments started on day 22 and continued for three weeks (dosing scheme in Table 21). AFP was measured once a week for 4 weeks, and BW was measured twice a week. At the end of the study tumor weights were also determined. QOD×9=one injection every other day for nine times; BID=twice daily; po=per oral.

TABLE 21
Dosing scheme - 8 mice per group

| | | | Dose | Dosing | | Schedule of | |
|---|---|---|---|---|---|---|---|
| Group | T reatment | Vehicle | [mg/kg] | volume | | administration* | Route |
| 1 | PB-S | PB-sucrose | — | 10 | ml/kg | QODx9 for3 weeks | iv |
| 2 | Sorafenib | — | 10 | 5 | ml/kg | BIDx30 | po |
| 3 | miR-Scr#1 | NOV340 | 6 | 10 | ml/kg | QODx9 for3 weeks | iv |
| 4 | miR-7 | NOV340 | 6 | 10 | ml/kg | QODx9 for3 weeks | iv |
| 5 | miR-34a | NOV340 | 6 | 10 | ml/kg | QODx9 for3 weeks | iv |
| 6 | miR-193a | NOV340 | 6 | 10 | ml/kg | QODx9 for3 weeks | iv |

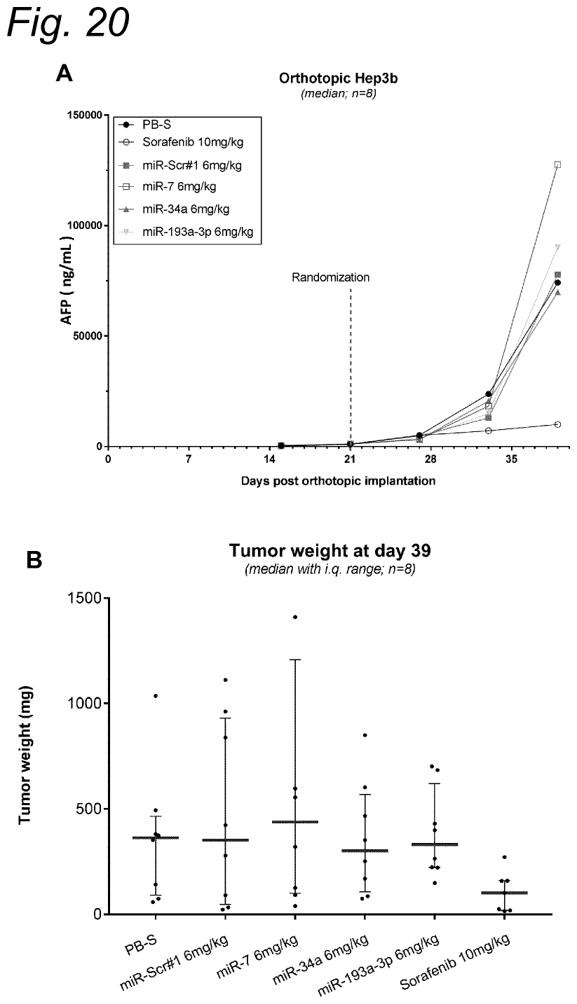

After terminal sacrifice on day 39, AFP levels and tumor weights were determined. NOV340 nanoparticles containing different miRNAs (miR-7; miR-34a or miR-193a-3p) did not inhibit growth of human Hep3b HCC (hepatocellular carcinoma) tumors measured by AFP and tumor weight, while Sorafenib (10 mg/kg, BID) did. These results are presented in FIG. 20.

Conclusion: Treatment with different NOV340 nanoparticle formulated miRNAs as compared to sorafenib are not able to inhibit tumor growth and/or weight.

### REFERENCES

Admadzada T. et al, Biophysical Reviews (2018) 10:69-86
Kearsley J. H., et al, 1990, PMID: 2372483
Knop et al., 2010, doi: 10.1002/anie.200902672
Loher et al., Oncotarget (2014) DOI: 10.18632/oncotarget.2405
Steffen P., Voss B., et al., Bioinformatics, 22:500-503, 2006
Wong, K. K. (PMID: 19149686)
Zhou et al., 2013, DOI: 10.1158/0008-5472.CAN-13-1094
EP17199997 WO2008/10558 U.S. Pat. Nos. 8,691,750 9,737,482 6,843,942

1. A composition comprising a nanoparticle, the nanoparticle comprising a diamino lipid and a miRNA or a source of a miRNA, wherein
i) the miRNA is a miRNA molecule, an isomiR, or a mimic thereof, and is an anticancer miRNA, wherein it is preferably an oligonucleotide with a seed sequence comprising at least 6 of the 7 nucleotides of the seed sequence represented by SEQ ID NOs: 17-50, and wherein said miRNA is preferably selected from the group consisting of miRNA-193a, miRNA-323, miRNA-342, miRNA-520f, miRNA-520f-i3, miRNA-3157, and miRNA-7, or an isomiR thereof, or a mimic thereof, and wherein
ii) the diamino lipid is of general formula (I) 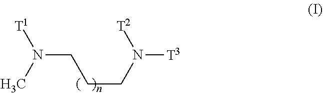
wherein
n is 0, 1, or 2, and
T¹, T², and T³ are each independently a C₁₀-C₁₈ chain with optional unsaturations and with zero, one, two, three, or four substitutions, wherein the substitutions are selected from the group consisting of C₁-C₄ alkyl, C₁-C₄ alkenyl, and C₁-C₄ alkoxy.

2. The composition according to claim 1, wherein said miRNA is
i) a miRNA-323-5p molecule, a miRNA-323-5p isomiR, or a miRNA-323-5p mimic, or
ii) a miRNA-342-5p molecule, a miRNA-324-5p isomiR, or a miRNA-324-5p mimic, or
iii) a miRNA-520f-3p molecule, a miRNA-520f-3p isomiR, or a miRNA-520f-3p mimic, or
iv) a miRNA-520f-3p-i3 molecule, a miRNA-520f-3p-i3 isomiR, or a miRNA-520f-3p-i3 mimic, or
v) a miRNA-3157-5p molecule, a miRNA-3157-5p isomiR, or a miRNA-3157-5p mimic, or
vi) a miRNA-193a-3p molecule, a miRNA-193a-3p isomiR, or a miRNA-193a-3p mimic, or
vii) a miRNA-7-5p molecule, a miRNA-7-5p isomiR, or a miRNA-7-5p mimic.

3. The composition according to claim 1, wherein a source of a miRNA is a precursor of a miRNA and is an oligonucleotide of at least 50 nucleotides in length.

4. The composition according to claim 1,
wherein said miRNA shares at least 70% sequence identity with any one of SEQ ID NOs: 51-125, 209, 211, 213, 215, 217, 219, or 221,
and/or wherein said miRNA is from 15-30 nucleotides in length,
and/or wherein said source of a miRNA is a precursor of said miRNA and shares at least 70% sequence identity with any one of SEQ ID NOs: 1-16, preferably with any one of SEQ ID NOs: 1-8.

5. The composition according to claim 1, further comprising a further miRNA or precursor thereof, wherein the miRNA is selected from the group consisting of miRNA-193a, miRNA-323, miRNA-342, miRNA-520f, miRNA-520f-i3, miRNA-3157, and miRNA-7, or an isomiR thereof, or a mimic thereof.

6. The composition according to claim 1, wherein the diamino lipid is of general formula (I) wherein T¹, T², and T³ are each independently selected from the group consisting of farnesyl, lauryl, tridecyl, myristryl, pentadecyl, cetyl, margaryl, stearyl, α-linolenyl, γ-linolenyl, linoleyl, stearidyl, vaccenyl, oleyl, elaidyl, palmitoleyl, and 3,7,11-trimethyldodecyl.

7. The composition according to claim 1, wherein the diamino lipid is of general formula (I) wherein n is 1.

8. The composition according to claim 1, wherein the diamino lipid is of general formula (I) wherein T¹, T², and T³ are identical.

9. The composition according to claim 1, further comprising a sterol, preferably selected from the group consisting of adosterol, brassicasterol, campesterol, cholecalciferol, cholestenedione, cholestenol, cholesterol, delta-7-stigmasterol, delta-7-avenasterol, dihydrotachysterol, dimethylcolesterol, ergocalciferol, ergosterol, ergostenol, ergostatrienol, ergostadienol, ethylcholestenol, fusidic acid, lanosterol, norcholestadienol, β-sitosterol, spinasterol, stigmastanol, stigmastenol, stigmastadienol, stigmastadienone, stigmasterol, and stigmastenone, more preferably cholesterol.

10. The composition according to claim 1, further comprising a phospholipid, preferably selected from the group consisting of distearoyl phosphatidylcholine (DSPC), dipalmitoyl phosphatidylcholine (DPPC), dimyristoyl phosphatidylcholine (DMPC), dilauroyl phosphatidylcholine (DLPC), dioleyl phosphatidylcholine (DOPC), 1,2-dioleoyl-sn-glycero-3-phosphoethanolamine (DOPE), egg phosphatidylcholine (EggPC), soy phosphatidylcholine (SoyPC), more preferably distearoyl phosphatidylcholine (DSPC).

11. The composition according to claim 1, further comprising a conjugate of a water soluble polymer and a lipophilic anchor, wherein:
i) the water soluble polymer is selected from the group consisting of poly(ethylene glycol) (PEG), poly(hydroxyethyl-1-asparagine) (PHEA), poly-(hydroxyethyl-L-glutamine) (PHEG), poly(glutamic acid) (PGA), polyglycerol (PG), poly(acrylamide) (PAAm), poly(vinylpyrrolidone) (PVP), poly(N-(2-hydroxypropyl)methacrylamide) (PHPMA), and poly(-oxazoline) (POx), preferably poly(ethylene glycol),
and wherein
ii) the lipophilic anchor is selected from the group consisting of a sterol, a lipid, and a vitamin E derivative.

12. The composition according to claim 1, wherein the nanoparticles comprise:
i) 20-60 mol % of diamino lipid, and
ii) 0-40 mol % of phospholipid, and
iii) 30-70 mol % of a sterol, and
iv) 0-10 mol % of a conjugate of a water soluble polymer and a lipophilic anchor as defined in claim 11.

13. The composition according to claim 1, wherein it is a pharmaceutical composition further comprising one or more pharmaceutically acceptable excipients.

14. A method for the treatment, prevention, delay, or amelioration of cancer comprising administering to a subject in need thereof
a miRNA or a source of a miRNA, wherein the miRNA is a miRNA molecule, an isomiR, or a mimic thereof, and is an anticancer miRNA, wherein it is preferably an oligonucleotide with a seed sequence comprising at least 6 of the 7 nucleotides of the seed sequence represented by SEQ ID NOs: 17-50, and wherein said miRNA is preferably selected from the group consisting of miRNA-193a, miRNA-323, miRNA-342, miRNA-520f, miRNA-520f-i3, miRNA-3157, and miRNA-7, or an isomiR thereof, or a mimic thereof; or
a pharmaceutical composition comprising a nanoparticle, the nanoparticle comprising said miRNA or said source of a miRNA and a diamino lipid, wherein
the diamino lipid is of general formula (I) 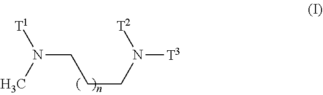
wherein
n is 0, 1, or 2, and
T¹, T², and T³ are each independently a C₁₀-C₁₈ chain with optional unsaturations and with zero, one, two, three, or four substitutions, wherein the substitutions are selected from the group consisting of C₁-C₄ alkyl, C₁-C₄ alkenyl, and C₁-C₄ alkoxy.

15. The method according to claim 14, wherein the miRNA or the source of the miRNA or the pharmaceutical composition is administered in an amount effective to downregulate an immunosuppressive tumour microenvironment in said subject.

16. The method according to claim 15, wherein the anticancer miRNA is miRNA-193a, or an isomiR thereof, or a mimic thereof, or a precursor thereof.

17. The method according to claim 14, wherein the miRNA or the source of the miRNA or the pharmaceutical composition is administered in an amount effective to promote or increase G2/M arrest in cancer cells in said subject.

18. An in vivo, in vitro, or ex vivo method for stimulating cellular uptake of a miRNA, the method comprising the step of contacting a cell with a composition as defined in claim 1.
